(19) United States
(12) Patent Application Publication
(10) Pub. No.: US 2009/0209571 A1
(43) Pub. Date: Aug. 20, 2009

(54) Phenanthrene derivatives as MPGES-1 inhibitors

(76) Inventors: Bernard Cote, L'lle-Perrot (CA); Yves Ducharme, Montreal (CA); Richard Frenette, Laval (CA); Richard Friesen, Kirkland (CA); Andre Giroux, Ste-Anne-De-Bellevue (CA); Evelyn Martins, Vaudreuil (CA); Pierre Hamel, Vimont-Laval (CA)

Correspondence Address: MERCK AND CO., INC, P O BOX 2000, RAHWAY, NJ, 07065-0907, US

(21) Appl. No.: 12/227,338

(22) PCT Filed: May 15, 2007
(86) PCT No.: PCT/CA2007/000867

§ 371 Date: Nov. 14, 2008

Related U.S. Application Data
(60) Provisional application No. 60/801,443, filed on May 18, 2006.

(51) Int. Cl. A61K 31/4188; C07D 495/04; C07D 487/04; C07D 471/04; A61K 31/437; A61K 31/4439; A61P 29/00

(52) U.S. Cl. 514/287; 548/301.7; 546/273.1; 546/64; 514/338; 514/394

(57) ABSTRACT

The invention encompasses novel compounds of Formula or pharmaceutically acceptable salts thereof. These compounds are inhibitors of the microsomal prostaglandin E synthase-1 (mPGES-1) enzyme and are therefore useful to treat pain and/or inflammation from a variety of diseases or conditions, such as osteoarthritis, rheumatoid arthritis and acute or chronic pain. Methods of treating diseases or conditions mediated by the mPGES-1 enzyme and pharmaceutical compositions are also encompassed.

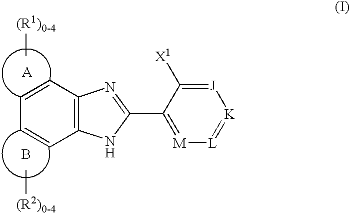

### BACKGROUND OF THE INVENTION

Modulation of prostaglandin metabolism is at the center of current anti-inflammatory therapies. NSAIDs and COX-2 inhibitors block the activity of cyclooxygenases and their ability to convert arachidonic acid (AA) into prostaglandin (PG) H2. PGH2 can be subsequently metabolized by terminal prostaglandin synthases to the corresponding biologically active PGs, namely, PGI2, thromboxane (Tx) A2, PGD2, PGF2α, and PGE2. A combination of pharmacological, genetic, and neutralizing antibody approaches demonstrates the importance of PGE2 in inflammation. In many respects, disruption of PGE2-dependent signalling in animal models of inflammation can be as effective as treatment with NSAIDs or COX-2 inhibitors. The conversion of PGH2 to PGE2 by prostaglandin E synthases (PGES) may therefore represent a pivotal step in the propagation of inflammatory stimuli.

Microsomal prostaglandin E synthase-1 (mPGES-1) is an inducible PGES after exposure to pro-inflammatory stimuli. mPGES-1 is induced in the periphery and in the CNS by inflammation and represents therefore a novel target for acute and chronic inflammatory disorders. The rationale for the development of specific mPGES-1 inhibitors revolves around the hypothesis that the therapeutic utility of NSAIDs and Cox-2 inhibitors would be largely due to inhibition of pro-inflammatory PGE2 while the side effect profile would be largely due to inhibition of other prostaglandins.

The present invention is directed to novel compounds that are selective inhibitors of the microsomal prostaglandin E synthase-1 enzyme and would therefore be useful for the treatment of pain and inflammation in a variety of diseases or conditions, such as osteoarthritis, rheumatoid arthritis and acute or chronic pain. Furthermore, by selectively inhibiting the pro-inflammatory PGE2, it is believed the compounds of the invention would have a reduced potential for side effects associated with the inhibition of other prostaglandins by conventional non-steroidal anti-inflammatory drugs, such as gastrointestinal and renal toxicity.

### SUMMARY OF THE INVENTION

The invention encompasses a genus of compounds of Formula I

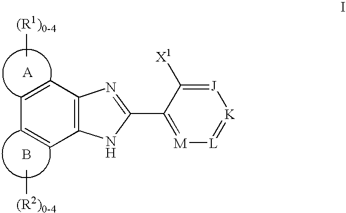

or pharmaceutically acceptable salts thereof. These compounds are inhibitors of the microsomal prostaglandin E synthase-1 (mPGES-1) enzyme and are therefore useful to treat pain and/or inflammation from a variety of diseases or conditions, such as osteoarthritis, rheumatoid arthritis and acute or chronic pain. Methods of treating diseases or conditions mediated by the mPGES-1 enzyme and pharmaceutical compositions are also encompassed.

### DETAILED DESCRIPTION OF THE INVENTION

The invention encompasses a genus of compounds represented by Formula I

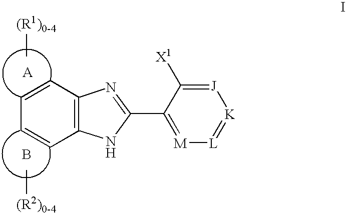

or a prodrug thereof, or a pharmaceutically acceptable salt of said compound or prodrug, wherein:
A is selected from the group consisting of: aryl, heteroaryl, heterocyclyl and cycloalkyl, or a fused analog of any of aforementioned;
B is selected from the group consisting of: aryl, heteroaryl, heterocyclyl and cycloalkyl, or a fused analog of any of the aforementioned;
with the proviso that A and B cannot both simultaneously be phenyl;
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—, with the proviso that at least one of J, K, L or M is other than —N—;
X¹ is selected from the group consisting of: (1) F; (2) Cl; (3) Br; (4) I; (5) —OH; (6) —N₃; (7) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (8) C₁₋₄alkoxy; (9) NR⁹R¹⁰—, NR⁹R¹⁰—C(O)—C₁₋₄alkyl-O— or NR⁹R¹⁰—C(O)—; (10) C₁₋₄alkyl-S(O)ₖ—; (11) —NO₂; (12) C₃₋₆cycloalkyl, (13) C₃₋₆cycloalkoxy; (14) phenyl, (15) carboxy; (16) C₁₋₄alkyl-O—C(O)—, and (17) —CN;
X², X³, X⁴ and X⁵ are independently selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy; (17) C₁₋₄alkyl-O—C(O)—, and (18) —CN;
each R¹ and R² is independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀alkyl or C₂₋₁₀alkenyl may be optionally substituted with one to three substituents independently selected from the group consisting of: —OH, acetyl, acetyloxy, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₁₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; (7) pyridyl and (8) pyridylmethyl; said C₁₋₄alkyl, C₃₋₆cycloalkyl, C₃₋₆cycloalkyl-C₁₋₄alkyl, phenyl, benzyl, pyridyl and pyridylmethyl may each be optionally substituted with 1 to 3 substituents independently selected from the group consisting of: OH, F, Cl, Br and I, and wherein said C₁₋₄alkyl may be further substituted with oxo or methoxy or both; each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2;
and when said compound of Formula I is a prodrug, said prodrug is represented by Formula A

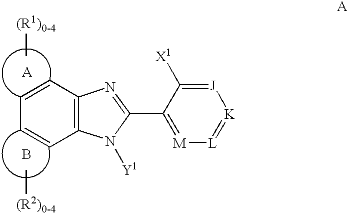

wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₁₋₄alkyl portion is optionally substituted with R³³—O—C(O)—; and (4) C₁₋₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.

Within the genus, the invention encompasses a first sub-genus of compounds represented by Formula B

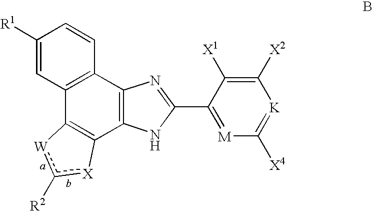

or a prodrug thereof, or a pharmaceutically acceptable salt of said compound or prodrug, wherein:
W is O or S, X is CR² and b is a double bond, or
X is O or S, W is CR² and a is a double bond.

Within the first sub-genus, the invention encompasses a class of compounds of Formula B wherein:

### X² and X⁴ are H;

### K is CH or N;

### M is —C(X⁵)—; and

X¹ and X⁵ are independently selected from the group consisting of: (1) F; (2) Cl; (3) Br; (4) I; and (5) CN.

Within the class, the invention encompasses a sub-class of compounds of Formula B wherein:

R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl,

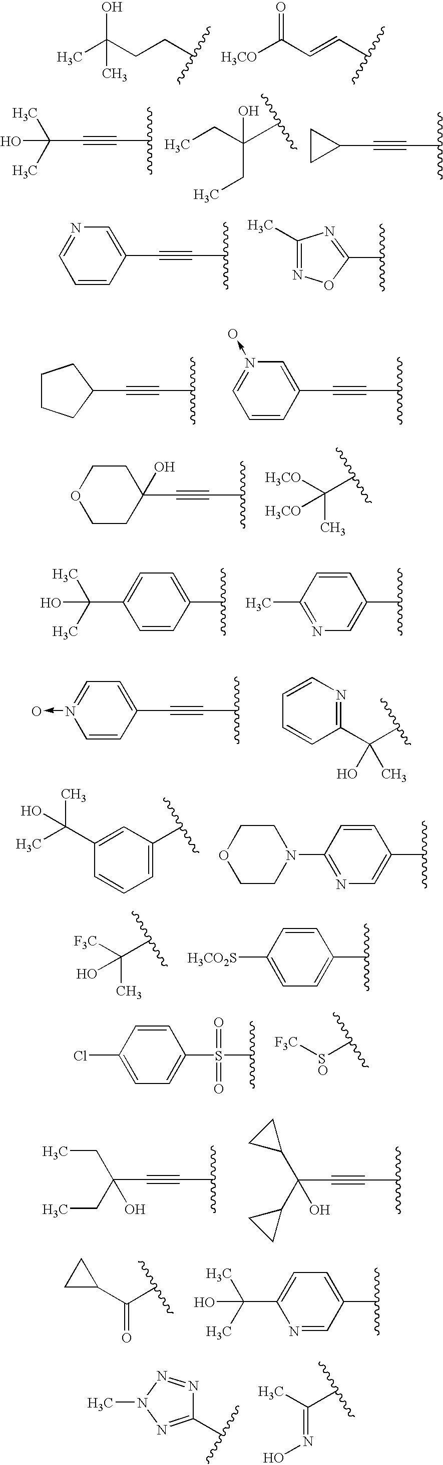

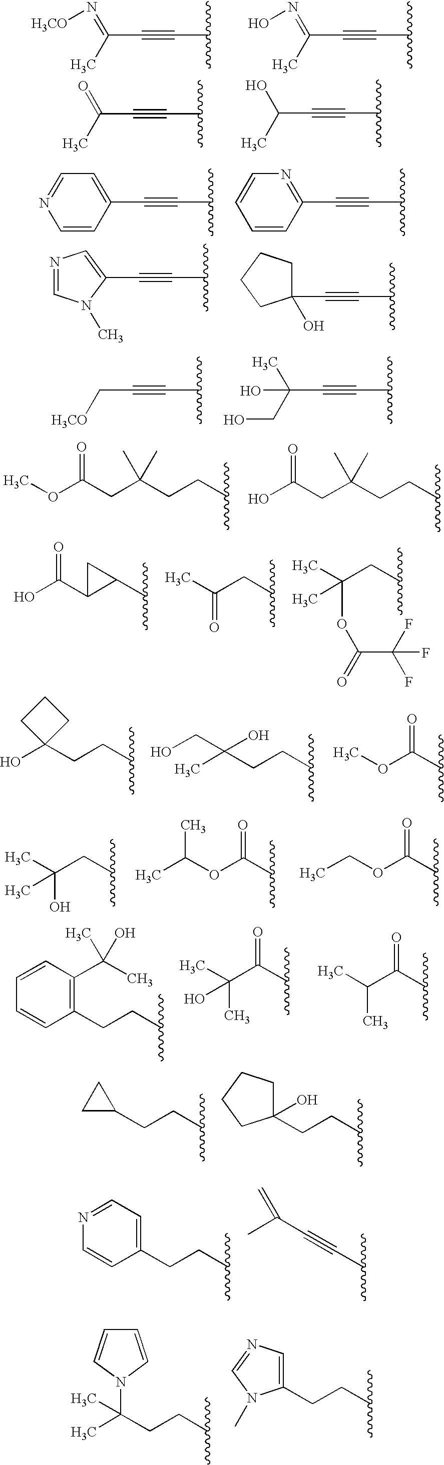

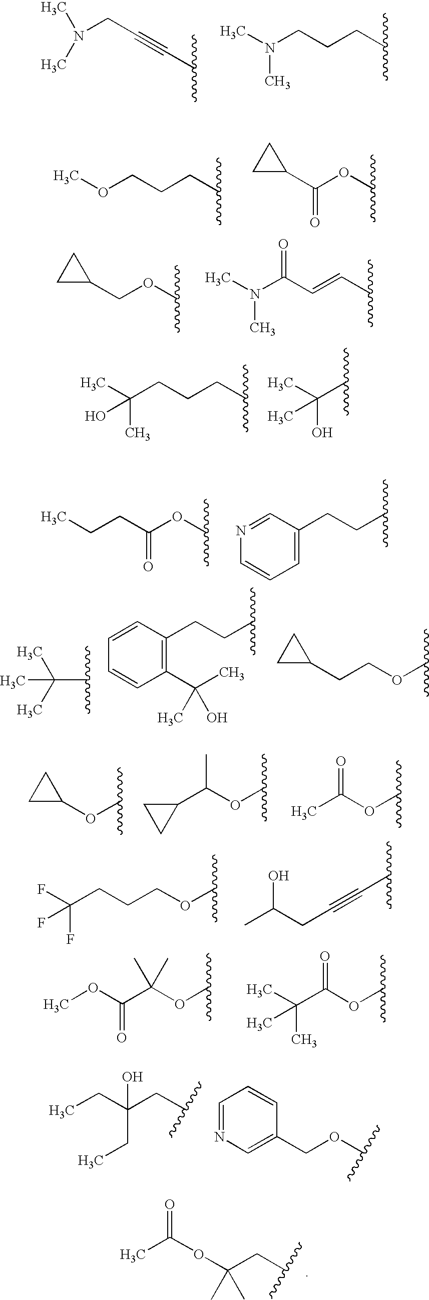

Within the sub-class, the invention encompasses a compound selected from the following table

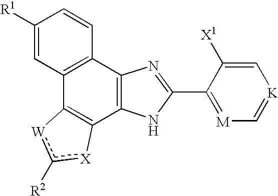

| W | X | R¹ | R² | X¹ | M | K |
|---|---|---|---|---|---|---|
| CH | S | H | H | Cl | CF | CH |
| S | CH | H | Cl | Cl | CF | CH |
| CH | O | H | H | Cl | CF | CH |
| S | CH | H | H | Cl | CF | CH |
| CH | S | CN | H | Cl | CF | CH |
| CH | S | Cl | H | Cl | CF | CH |
| CH | S | Cl | H | CN | CF | CH |
| CH | S | Cl | H | Br | CBr | CH |
| CH | S | Cl | H | CN | CCN | CH |
| CH | S | Cl | H | Br | CBr | N |
| CH | S | Cl | H | CN | CCN | N |
| CH | O | Cl | H | CN | CCN | CH |
| CH | O | Cl | H | Br | CBr | N |
| CH | O | Cl | H | CN | CCN | N |
| CH | S | Cl | Br | Cl | CF | CH |
| S | CH | H | Br | Cl | CF | CH |
| CH | S | Cl | Br | CN | CCN | CH |
| CH | S | Cl | 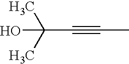 | CN | CCN | CH |
| CBr | S | Cl | Br | Br | CBr | CH |
| CH | S | Cl | Cl | CN | CCN | CH |
| CH | S | Br | Cl | Br | CBr | CH |
| CH | S | Br | Cl | CN | CCN | CH |
| CH | S | 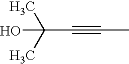 | Cl | CN | CCN | CH |
| CBr | S | Cl | Cl | Br | CBr | CH |
| CBr | S | Cl | Cl | CN | CCN | CH |
| CCl | S | Br | Cl | Br | CBr | CH |
| CCl | S | Br | Cl | CN | CCN | CH |
| CCl | S | 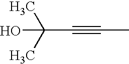 | Cl | CN | CCN | CH |
| CCl | S | 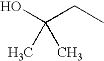 | Cl | CN | CCN | CH |
| CH | S | 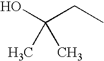 | Cl | CN | CCN | CH |
| CH | S | 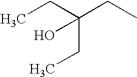 | Cl | CN | CCN | CH |

or a pharmaceutically acceptable salt of any of the aforementioned compounds.

Also within the genus, the invention encompasses a second sub-genus of compounds represented by Formula C

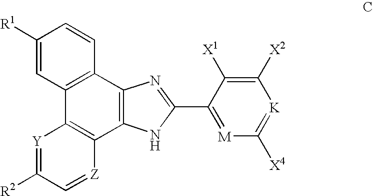

wherein Y and Z are independently selected from the group consisting of: CH and N, or the N-oxide thereof.

Within the second sub-genus, the invention encompasses a class of compounds represented by Formula C wherein

### X² and X⁴ are H;

### K is CH or CF;

### M is —C(X⁵)—; and

X¹ and X⁵ are independently selected from the group consisting of: (1) F; (2) Cl; (3) Br; (4) I; and (5) CN.

Within the class, the invention encompasses a sub-class of compounds represented by Formula C wherein

R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl,

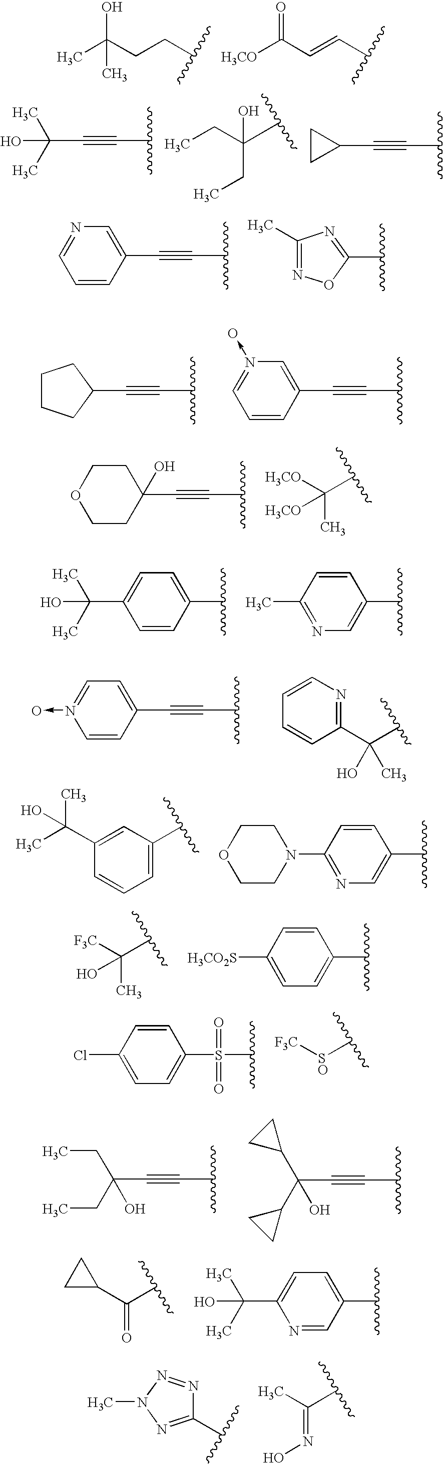

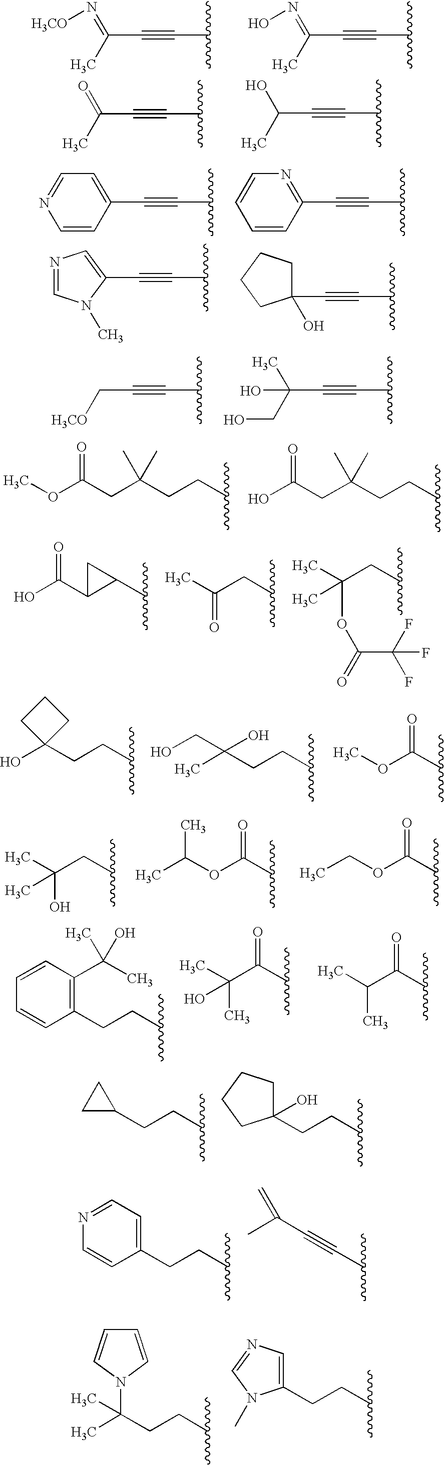

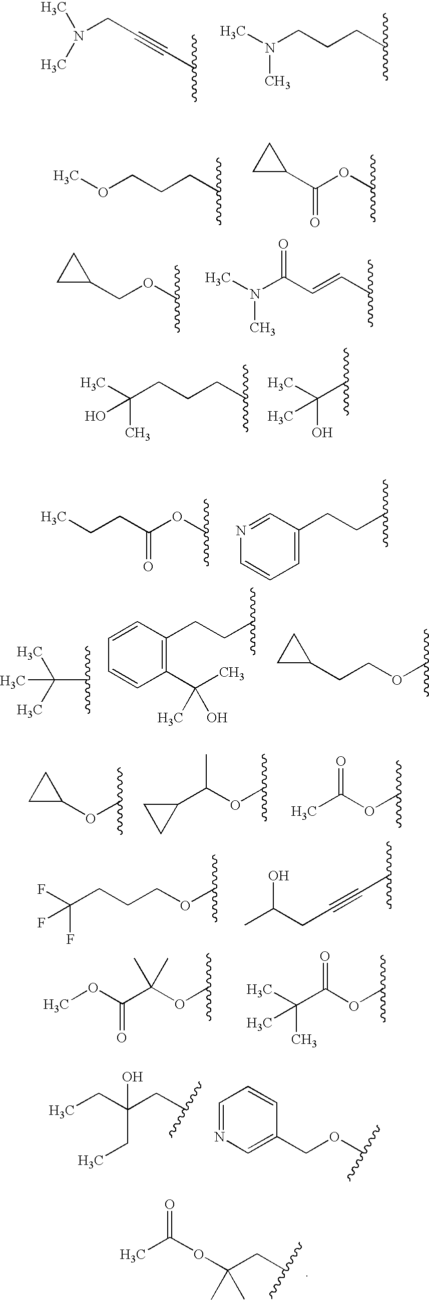

Within the sub-class the invention encompasses a compound selected from the following table

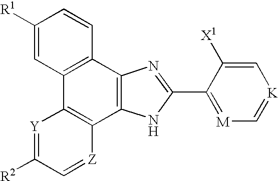

| Y | Z | R¹ | R² | X¹ | M | K |
|---|---|---|---|---|---|---|
| CH | N | H | H | Cl | CF | CH |
| N | CH | H | H | Cl | CF | CH |
| NO | CH | H | H | Cl | CF | CH |
| NH | CH | H | O | Cl | CF | CH |
| N | CH | H | H | Br | CBr | CH |
| N | CH | H | H | CN | CCN | CH |
| NO | CH | H | H | Br | CBr | CH |
| N | CH | H | Cl | CN | CCN | CH |
| N | CH | H | Ph | Br | CBr | CH |
| N | CH | H | Ph | CN | CCN | CH |
| N | CH | OCH3 | Cl | Br | CBr | CH |
| N | CH | OCH3 | Cl | CN | CCN | CH |
| N | CH | 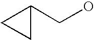 | 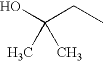 | Br | CBr | CH |
| N | CH | 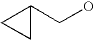 | 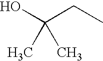 | CN | CCN | CH |
| N | CH | 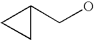 | Cl | CN | CCN | CH |
| N | CH | 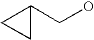 | 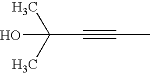 | CN | CCN | CH |
| N | CH | 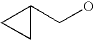 | 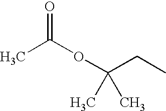 | CN | CCN | CH |
| N | CH | 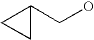 | CN | CN | CCN | CH |
| N | CH | 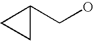 | 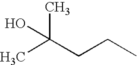 | CN | CCN | CH |
| N | CH | 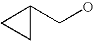 | Cl | CN | CCN | CF |
| N | CH | 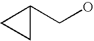 | 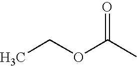 | CN | CCN | CH |
| N | CH | 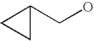 | 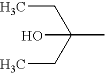 | CN | CCN | CH |
| N | CH | 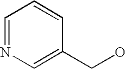 | Cl | CN | CCN | CH |
| N | CH | 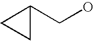 | 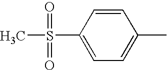 | CN | CCN | CH |

or a pharmaceutically acceptable salt of any of the aforementioned compounds.

Also with the genus, the invention encompasses a third sub-genus of compounds represented by Formula I wherein:

X¹ is selected from the group consisting of: (1) F; (2) Cl; (3) Br; (4) I; and (5) CN; and
X², X³, X⁴ and X⁵ are independently selected from the group consisting of: (1) H; (2) F; (3) Cl; (4) Br; (5) I; and (6) CN.

Also within the genus, the invention encompasses a fourth sub-genus of compounds represented by Formula I wherein M is —C(X⁵).

Within the fourth sub-genus, the invention encompasses a class of compounds represented by Formula I wherein X⁵ is other than H.

Within the class, the invention encompasses a sub-class of compounds represented by Formula I wherein X¹ and X⁵ are the same and selected from the group consisting of: (1) F; (2) Cl; (3) Br; (4) I and (5) CN.

Also within the genus, the invention encompasses a fifth sub-genus of compounds represented by Formula I wherein at least one of R¹ or R² is present and other than H.

Within the fifth sub-genus, the invention encompasses a class of compound represented by Formula I wherein: R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl

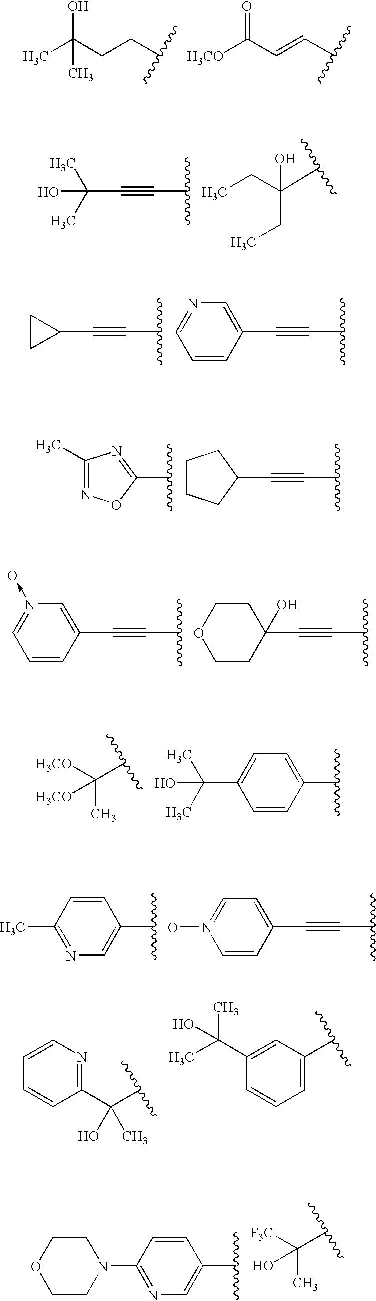

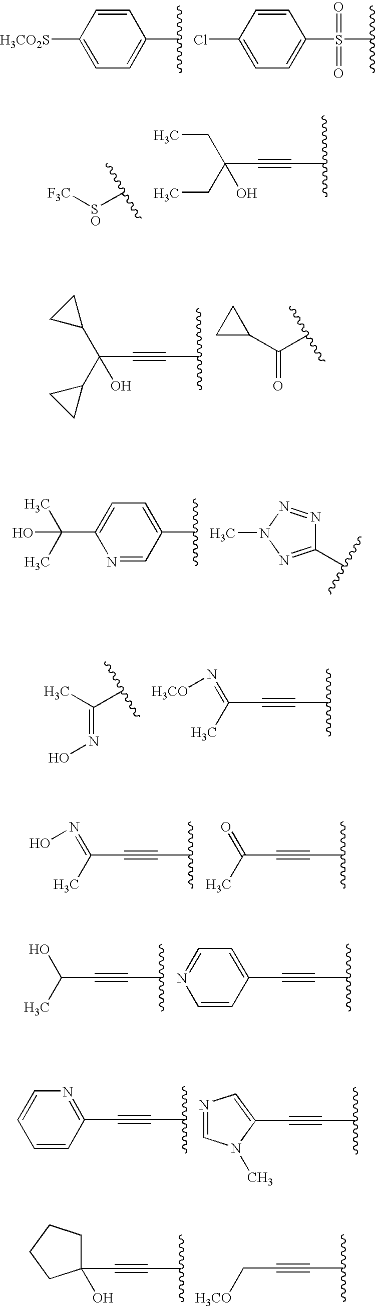

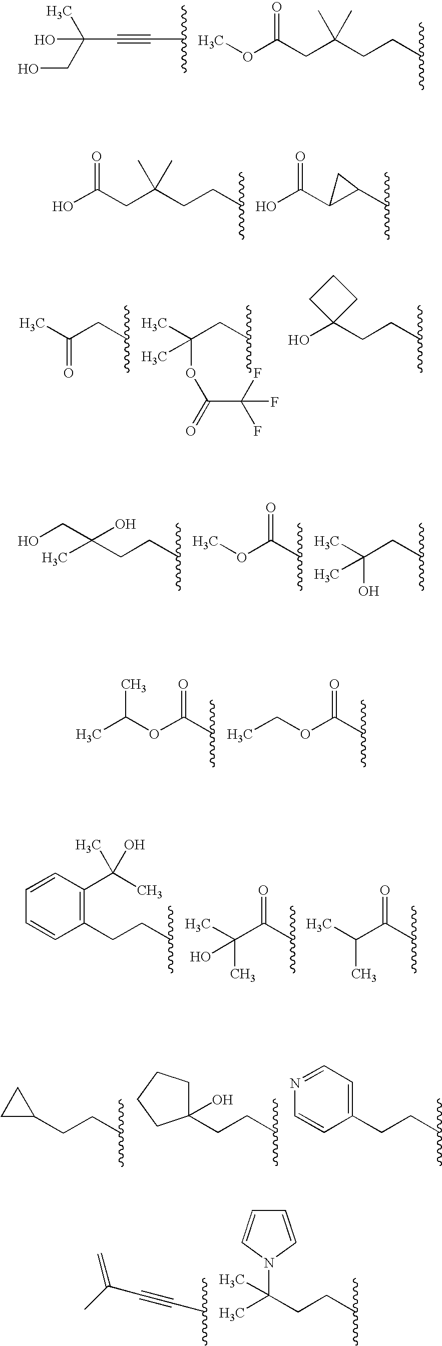

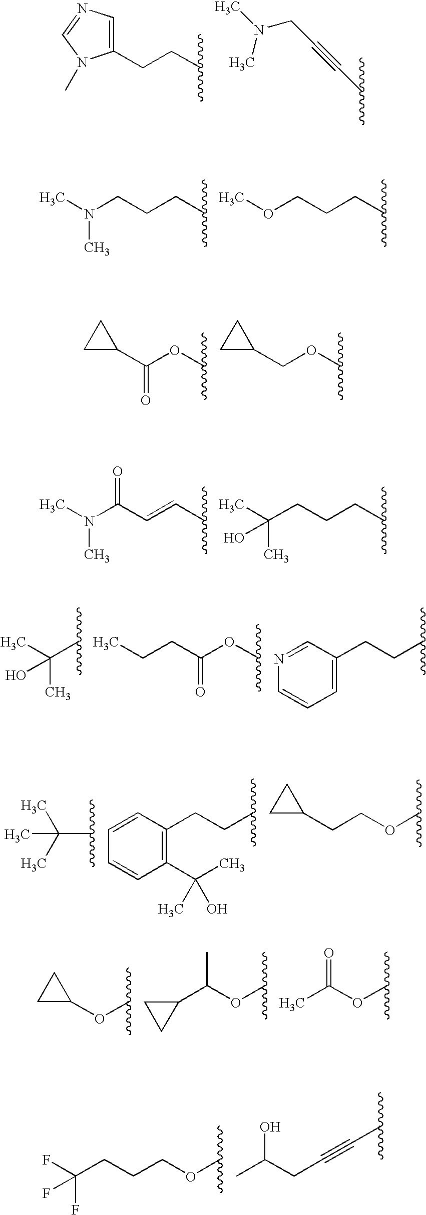

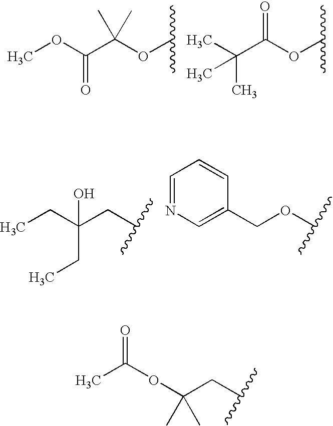

with the proviso that at least one of R¹ or R² is present and other than H.

Also within the genus, the invention encompasses a sixth sub-genus of compounds of Formula I wherein

A is selected from the group consisting of: aryl and heteroaryl, or a fused analog of any of aforementioned;
B is selected from the group consisting of: aryl and heteroaryl, or a fused analog of any of the aforementioned;
with the proviso that A and B cannot both simultaneously be phenyl.

Also within the genus, the invention encompasses a compound selected from the following group:

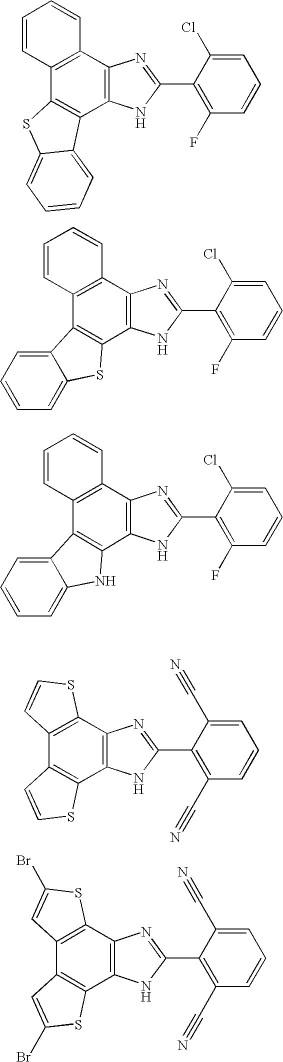

or a pharmaceutically acceptable salt of any of the aforementioned compounds.

In another embodiment, the invention encompasses a pharmaceutical composition comprising a compound of Formula I in combination with a pharmaceutically acceptable carrier.

In another embodiment, the invention encompasses a method for treating a microsomal prostaglandin E synthase-1 mediated disease or condition in a human patient in need of such treatment comprising administering to said patient a compound of Formula I in an amount effective to treat the microsomal prostaglandin E synthase-1 mediated disease or condition.

In the tables described herein, the bonds to the ring are shown for R² in Ex. 18, R¹ in Ex. 23, R¹ in Exs. 28 to 31, R² in Exs. 44 and 45, R² in Exs. 47 to 53 and R² in Ex. 55. See as an illustration Example 53.

Within this embodiment, the invention encompasses the above described method wherein the disease or condition is selected from the group consisting of: acute or chronic pain, osteoarthritis, rheumatoid arthritis, bursitis, ankylosing sponylitis and primary dysmenorrhea.

When R¹ or R² are not present or present in a number which is less to fulfill the valency requirements of the various atoms, hydrogen atoms are present to fulfill the valency requirements of the atoms.

The invention includes, as appropriate, pharmaceutically acceptable salts of any of the aforementioned compounds. For purposes of this specification, the heading “R³/R⁶” means that the substituent indicated in that column is substituted at the position represented by either R³ or R⁶. In the adjacent column, the heading “R⁶/R³” means the indicated substituent is substituted at the position R³ or R⁶ not substituted in the previous column. By way of example, Example 6 represents R³═CN and R⁶═H or R³═H and R⁶═CN, representing both tautomers.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes, propenyl, 1-methylethenyl, butenyl and the like.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

“Cycloalkyl” means mono- or bicyclic saturated carbocyclic rings, each of which having from 4 to 8 carbon atoms. A “fused analog” of cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from O, S or N (or the N-oxide thereof), with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Heterocyclyl” means mono- or bicyclic saturated rings containing at least one heteroatom selected from O, S or N (or the N-oxide thereof), each of said ring having from 4 to 8 atoms in which the point of attachment may be carbon or nitrogen. A “fused analog” of heterocyclyl means a monocyclic heterocycle fused to an aryl or heteroaryl group in which the point of attachment is on the non-aromatic portion. Examples of “heterocyclyl” and fused analogs thereof include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. The compound of Formula I exists in the following tautomeric forms:

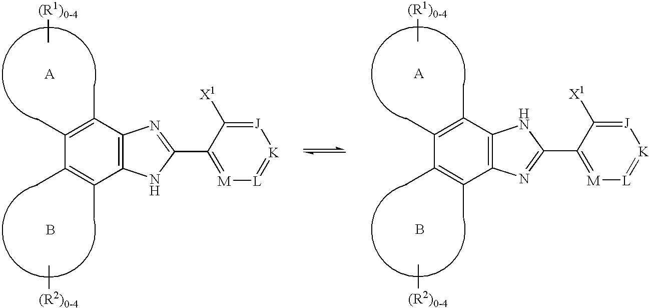

The individual tautomers as well as mixture thereof are encompassed within Formula I.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu. Exemplifying prodrugs of the invention are compounds of Formula C.

The term “treating a microsomal prostaglandin E synthase-1 mediated disease or condition” means treating or preventing any disease or condition that is advantageously treated or prevented by inhibiting the microsomal prostaglandin E synthase-1 (mPGES-1) enzyme. The term includes the relief of pain, fever and inflammation of a variety of conditions including rheumatic fever, symptoms associated with influenza or other viral infections, common cold, low back and neck pain, dysmenorrhea, headache, migraine (acute and prophylactic treatment), toothache, sprains and strains, myositis, neuralgia, synovitis, arthritis, including rheumatoid arthritis, degenerative joint diseases (osteoarthritis), gout and ankylosing spondylitis, acute, subacute and chronic musculoskeletal pain syndromes such as bursitis, burns, injuries, and pain following surgical and dental procedures as well as the preemptive treatment of surgical pain. In addition, the term includes the inhibition cellular neoplastic transformations and metastic tumor growth and hence the treatment of cancer. The term also includes the treatment of endometriosis and Parkinson's disease as well as the treatment of mPGES-1 mediated proliferative disorders such as may occur in diabetic retinopathy and tumor angiogenesis. The term “treating” encompasses not only treating a patient to relieve the patient of the signs and symptoms of the disease or condition but also prophylactically treating an asymptomatic patient to prevent the onset or progression of the disease or condition.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. Suitable dosage levels of the compound of Formula I used in the present invention are described below. The compound may be administered on a regimen of once or twice per day.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” include salts prepared from bases that result in non-toxic pharmaceutically acceptable salts, including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from acids that result in pharmaceutically acceptable salts, including inorganic and organic acids. Such acids include acetic, adipic, aspartic, 1,5-naphthalenedisulfonic, benzenesulfonic, benzoic, camphorsulfonic, citric, 1,2-ethanedisulfonic, ethanesulfonic, ethylenediaminetetraacetic, fumaric, glucoheptonic, gluconic, glutamic, hydriodic, hydrobromic, hydrochloric, isethionic, lactic, maleic, malic, mandelic, methanesulfonic, mucic, 2-naphthalenesulfonic, nitric, oxalic, pamoic, pantothenic, phosphoric, pivalic, propionic, salicylic, stearic, succinic, sulfuric, tartaric, p-toluenesulfonic acid, undecanoic, 10-undecenoic, and the like.

By virtue of the mPGES-1 inhibitory activity of compounds of the present invention, the compounds of Formula I are useful for the relief of pain, fever and inflammation of a variety of conditions including rheumatic fever, symptoms associated with influenza or other viral infections, common cold, low back and neck pain, dysmenorrhea, headache, migraine (acute and prophylactic treatment), toothache, sprains and strains, myositis, neuralgia, synovitis, arthritis, including rheumatoid arthritis, juvenile rheumatoid arthritis, degenerative joint diseases (osteoarthritis), acute gout and ankylosing spondylitis, acute, subacute and chronic musculoskeletal pain syndromes such as bursitis, burns, injuries, and pain following surgical and dental procedures as well as the preemptive treatment of surgical pain. In addition, such a compound may inhibit cellular neoplastic transformations and metastic tumor growth and hence can be used in the treatment of cancer. Compounds of Formula I may also be useful for the treatment or prevention of endometriosis, hemophilic arthropathy and Parkinson's disease.

Compounds of Formula I will also inhibit prostanoid-induced smooth muscle contraction by preventing the synthesis of contractile prostanoids and hence may be of use in the treatment of dysmenorrhea, premature labor and asthma.

By virtue of their selective inhibition of the mPGES-1 enzyme, the compounds of Formula I will prove useful as an alternative to conventional non-steroidal antiinflammatory drugs (NSAID'S) particularly where such non-steroidal antiinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anemia such as hypoprothrombinemia, haemophilia or other bleeding problems (including those relating to reduced or impaired platelet function); kidney disease (e.g., impaired renal function); those prior to surgery or taking anticoagulants; and those susceptible to NSAID induced asthma.

The compounds of the invention are also useful for treating or preventing a neoplasia in a subject in need of such treatment or prevention. The term “treatment” includes partial or total inhibition of the neoplasia growth, spreading or metastasis, as well as partial or total destruction of the neoplastic cells. The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia. The term “subject” for purposes of treatment includes any human or mammal subject who has any one of the known neoplasias, and preferably is a human subject. For methods of prevention, the subject is any human or animal subject, and preferably is a human subject who is at risk for obtaining a neoplasia. The subject may be at risk due to exposure to carcinogenic agents, being genetically predisposed to have the neoplasia, and the like.

The term “neoplasia” includes both benign and cancerous tumors, growths and polyps. Thus, the compounds of the invention are useful for treating or preventing benign tumors, growths and polyps including squamous cell papilloma, basal cell tumor, transitional cell papilloma, adenoma, gastrinoma, cholangiocellular adenoma, hepatocellular adenoma, renal tubular adenoma, oncocytoma, glomus tumor, melanocytic nevus, fibroma, myxoma, lipoma, leiomyoma, rhabdomyoma, benign teratoma, hemangioma, osteoma, chondroma and meningioma. The compounds of the invention are also useful for treating or preventing cancerous tumors, growths and polyps including squamous cell carcinoma, basal cell carcinoma, transitional cell carcinoma, adenocarcinoma, malignant gastrinoma, cholangiocelleular carcinoma, hepatocellular carcinoma, renal cell carcinoma, malignant melanoma, fibrosarcoma, myxosarcoma, liposarcoma, leimyosarcoma, rhabdomyosarcoma, malignant teratoma, hemangiosarcoma, Kaposi sarcoma, lymphangiosarcoma, ostreosarcoma, chondrosarcoma, malignant meningioma, non-Hodgkin lymphoma, Hodgkin lymphoma and leukemia. For purposes of this specification, “neoplasia” includes brain cancer, bone cancer, epithelial cell-derived neoplasia (epithelial carcinoma), basal cell carcinoma, adenocarcinoma, gastrointestinal cancer such as lip cancer, mouth cancer, esophogeal cancer, small bowel cancer and stomach cancer, colon cancer, rectal cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that affect epithelial, mesenchymal or blood cells throughout the body. The compounds of the invention are useful for treating or preventing any of the aforementioned cancers. The compounds of the invention are useful for treating or preventing benign and cancerous tumors, growths and polyps of the following cell types: squamous epithelium, basal cells, transitional epithelium, glandular epithelium, G cells, bile ducts epithelium, hepatocytes, tubules epithelium, melanocytes, fibrous connective tissue, cardiac skeleton, adipose tissue, smooth muscle, skeletal muscle, germ cells, blood vessels, lymphatic vessels, bone, cartilage, meninges, lymphoid cells and hematopoietic cells. The compounds can be used to treat subjects having adenomatous polyps, including those with familial adenomatous polyposis (FAP). Additionally, the compounds can be used to prevent polyps from forming in patients at risk of FAP. Preferably, the compounds of the invention are useful for treating or preventing the following cancers: colorectal, esophagus stomach, breast, head and neck, skin, lung, liver, gall bladder, pancreas, bladder, endometrium cervix, prostate, thyroid and brain.

Similarly, compounds of Formula I will be useful as a partial or complete substitute for conventional NSAIDs in preparations wherein they are presently co-administered with other agents or ingredients. Thus in further aspects, the invention encompasses pharmaceutical compositions for treating mPGES-1 mediated diseases as defined above comprising a non-toxic therapeutically effective amount of the compound of Formula I as defined above and one or more ingredients such as another pain reliever including acetaminophen or phenacetin; opioid analgesics, such as codeine, fentanyl, hydromorphone, levorphanol, meperidine, methadone, morphine, oxycodone, oxymorphine, propoxyphene, buprenorphine, butorphanol, dezocine, nalbuphine and pentazocine; a potentiator including caffeine; an H2-antagonist; aluminum or magnesium hydroxide; simethicone; a decongestant including phenylephrine, phenylpropanolamine, pseudophedrine, oxymetazoline, ephinephrine, naphazoline, xylometazoline, propylhexedrine, or levo-desoxyephedrine; an antitussive including codeine, hydrocodone, caramiphen, carbetapentane, or dextramethorphan; a diuretic; a sedating or non-sedating antihistamine; a proton pump inhibitor, such as omeprazole; a bradykinin-1 antagonist; a VR1 receptor antagonist; and a sodium channel blocker (NAV1). For the treatment or prevention of migraine, the invention also encompasses co-administration with a 5-HT agonist such as rizatriptan, sumatriptan, zolmitriptan and naratriptan, or a CGRP antagonist. In addition the invention encompasses a method of treating mPGES-1 mediated diseases comprising: administration to a patient in need of such treatment a non-toxic therapeutically effect amount of the compound of Formula I, optionally co-administered with one or more of such ingredients as listed immediately above.

As indicated above, pharmaceutical compositions for treating mPGES-1 mediated diseases as defined may optionally include one or more ingredients as listed above.

In another aspect, the invention encompasses co-administering a proton pump inhibitor with a compound of Formula I. The proton pump inhibitors that may be utilized in this aspect of the invention include omeprazole, lansoprazole, rabeprazole, pantoprazole, and esomeprazole, or a pharmaceutically acceptable salt of any of the aforementioned. These proton pump inhibitors are commercially available, e.g., omeprazole (PRILOSEC, AstraZeneca), lansoprazole (PREVACID, TAP Pharmaceuticals), rabeprazole (ACIPHEX, Janssen Pharmaceutica), pantoprazole (PROTONIX, Wyeth-Ayerst), and esomeprazole (NEXIUM, AstraZeneca). The said proton pump inhibitors may be administered at conventional doses. For example, omeprazole or omeprazole magnesium may be administered at a dose of 10 mg, 20 mg or 40 mg. Lansoprazole may be administered at a dose of 15 mg or 30 mg. Rabeprazole sodium may be administered at a dose of 20 mg. Pantoprazole may be administered at a dose of 20 mg or 40 mg. Esomeprazole may be administered at a dose of 20 mg or 40 mg. The compound of Formula I and the proton pump inhibitor may be administered concomitantly in a single pharmaceutical dosage form or as two separate dosage forms taken by a patient substantially at the same time. Alternatively, the compound of Formula I and the proton pump inhibitor may be taken sequentially at separately staggered times as long as the pharmaceutical effects of the two agents are being realized by the patient at the same time.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil. Exemplifying a formulation for the present invention is a dry filled capsule containing a 50/50 blend of microcrystalline cellulose and lactose and 1 mg, 10 mg or 100 mg of the compound of Formula I.

Aqueous suspensions contain the active material in admixture with excipients suitable for the manufacture of aqueous suspensions. Such excipients are suspending agents, for example sodium carboxymethyl-cellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Liquid formulations include the use of self-emulsyfying drug delivery systems and NanoCrystal® technology. Cyclodextrin inclusion complexes can also be utilized.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I are employed. (For purposes of this application, topical application shall include mouth washes and gargles.)

Pharmaceutical compositions of the invention may also utilize absorption enhancers such as tween 80, tween 20, Vitamin E TPGS (d-alpha-tocopheryl polyethylene glycol 1000 succinate) and Gelucire®.

Dosage levels of the order of from about 0.01 mg to about 140 mg/kg of body weight per day are useful in the treatment of the above-indicated conditions, or alternatively about 0.5 mg to about 7 g per patient per day. For example, inflammation may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg. Dosage amounts of 4 mg, 8 mg, 18 mg, 20 mg, 36 mg, 40 mg, 80 mg, 160 mg, 320 mg and 640 mg may also be employed. Dosage unit forms containing 1, 10 or 100 mg are also encompassed.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

### Methods of Synthesis

### 1) Thiophene/Oxazole Series

The compounds of Formula Ia-d of the present invention can be prepared according to the synthetic routes outlined in Schemes 1 to 5 below and by following the methods described therein. The imidazole of Formula Ia-b may be prepared in a multi-step sequence from the requisite heterophenanthrenequinone ia. The heterophenanthrene imidazole Ia is obtained by treating the heterophenanthrenequinone ia and an appropriately substituted aldehyde ii with a reagent such as NH₄OAc or NH₄HCO₃ in a solvent such as acetic acid. Imidazole Ia could be further elaborated to imidazole Ib by the appropriate interconversion of any of the functional groups R₁ to R₅, such as halogenation with a suitable halogenating agent such as bromine or N-bromosuccinimide, in a suitable solvent such as THF or acetic acid, or transmetallation with an organometallic reagent, such as butyl lithium, in a suitable solvent such as THF, followed by the addition of an electrophile, such as iodine or carbon dioxide, or any other appropriate functional group interconversion. Treatment of the imidazoles Ia or Ib with CuCN in a solvent such as DMF or DMSO produces the mono or bis-nitrile (M=CCN) Ic. Subsequent functional group interconversion can be done at any of the R₁ to R₅ positions. For example, if one or more of the R₁ to R₅ substituents equal Cl, Br or I and if M is different from CBr or CI, Ic could be converted to Id by placing Ic in the presence of a monosubstituted alkynyl, a stannane, a boronic acid, a borane or a boronate under conditions that promote cross coupling reactions, such as heating in the presence of a catalyst, such as Pd(PPh₃)₄ and CuI, in the presence of a base, such as sodium carbonate or diisopropylamine, and in a suitable solvent, such as THF, DMF or DME. This last exemplified step, or any other appropriate functional group transformation, can be iteratively repeated on R₁ to R₅.

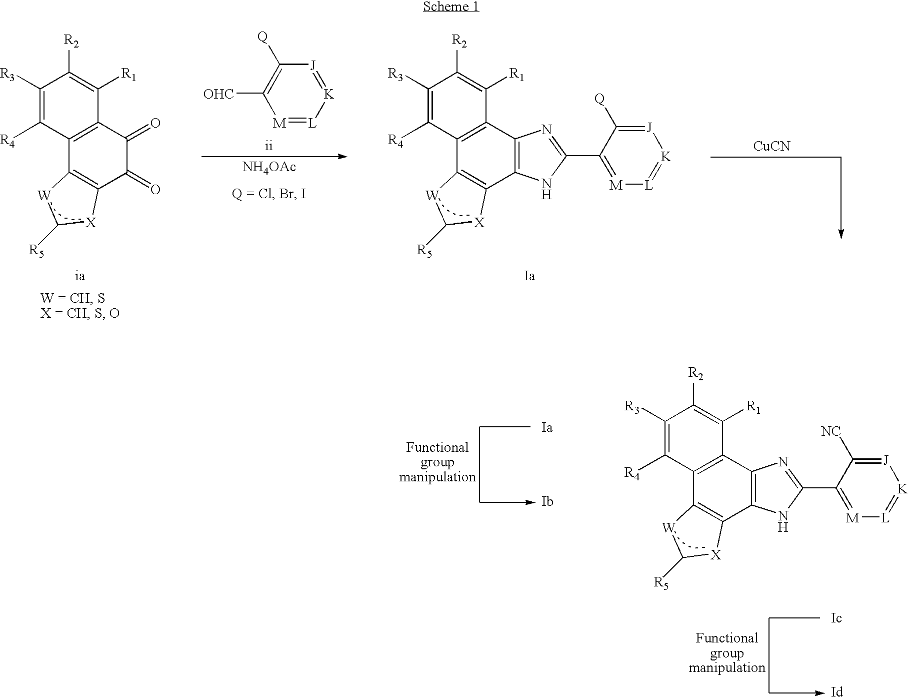

Heterophenanthrenequinone ia can be prepared according to the sequences outlined in Schemes 2 and 3. As shown in Scheme 2, treatment of an appropriately substituted bromo-phenylacetic ester iii (which could be prepared for example, by esterification of phenylacetic acids, displacement of activated aryl fluorides with malonate derivatives followed by decarboxylation, or Wolffe rearrangement of benzoic acids) with a heteroaryl boronic acid iv in the presence of a catalyst such as Pd(PPh₃)₄ and in the presence of a base, such as cesium fluoride, in an suitable solvent, such as DMF or DME, followed by hydrolysis of the ester with a base such as sodium hydroxide in a suitable mixture of solvents such as THF and methanol produces the phenylacetic acid v. Conversion of the acid v into its acyl chloride upon treatment with a suitable reagent such as thionyl chloride or oxalyl chloride in the presence of a catalytic amount of DMF in a solvent such as 1,2-dichloroethane or THF, followed by intramolecular cyclisation in the presence of a Lewis acid such as aluminum trichloride in a solvent such as 1,2-dichloroethane produces the phenanthrol vi. This phenanthrol vi can be directly oxidized to heterophenanthrenequinone ia by treatment with a catalytic amount of N,N′-Bis(salicylidene)ethylenediaminocobalt (II) hydrate [Co(SALEN)₂] in a solvent such as DMF, in the presence of air. Alternatively, bromo-phenylacetic ester iii could be converted into its boronate ester vii by treatment with bis(pinacolato)diboron in the presence of a catalyst such as Pd(dppf)Cl₂ and in the presence of a base such as potassium acetate, in a suitable solvent such as DMF. Cross-coupling of this boronate ester vii with a heteroaryl bromide viii in the presence of a catalyst such as Pd(dppf)Cl₂ and in the presence of a base such as potassium acetate, in a suitable solvent such as DMF produces the phenylacetic acid v, which can then be elaborated to heterophenanthrenequinone ia as described above.

Heterophenanthrenequinone ia can also be prepared as shown in Scheme 3. Deprotonation of the phosphonium salt ix in the presence of a base, such as sodium hydride or sodium methoxide, in a solvent such as DMF followed by addition of the aldehyde x produces the stillbene xi as a mixture of E and Z isomers. Intramolecular cyclisation of this mixture upon exposition to UV light in the presence of an oxidizing agent, such as iodine, and an acid scavenger, such as propylene oxide, in a suitable solvent such as cyclohexane produces the heterophenanthrene xiia. This heterophenanthrene xiia can be directly oxidized with an oxidizing agent, such as CrO₃, in a suitable solvent, such as acetic acid, to provide the heterophenanthrenequinone ia, or optionally, heterophenanthrene xiia could be further elaborated to heterophenanthrene xiib (as exemplified in Schemes 4 and 5) by the appropriate interconversion of any of the functional groups R₁ to R₅, such as halogenation with a reagent such as N-bromosuccinimide or transmetallation with an organometallic reagent, such as butyl lithium, in a suitable solvent such as THF, followed by the addition of an electrophile, such as iodine or carbon dioxide.

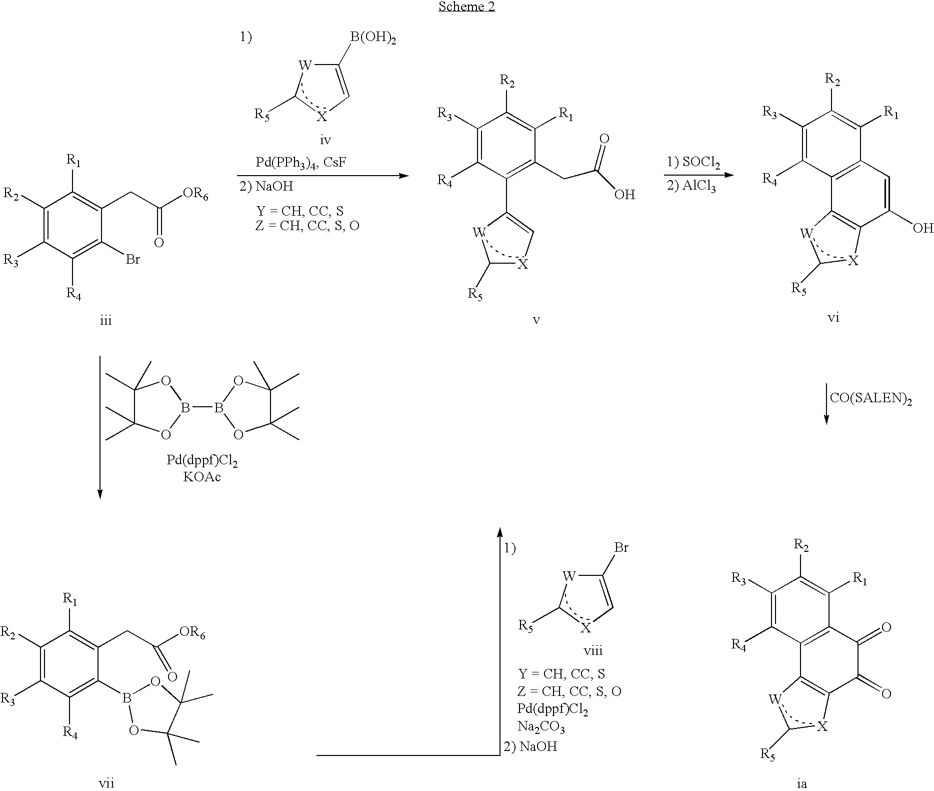

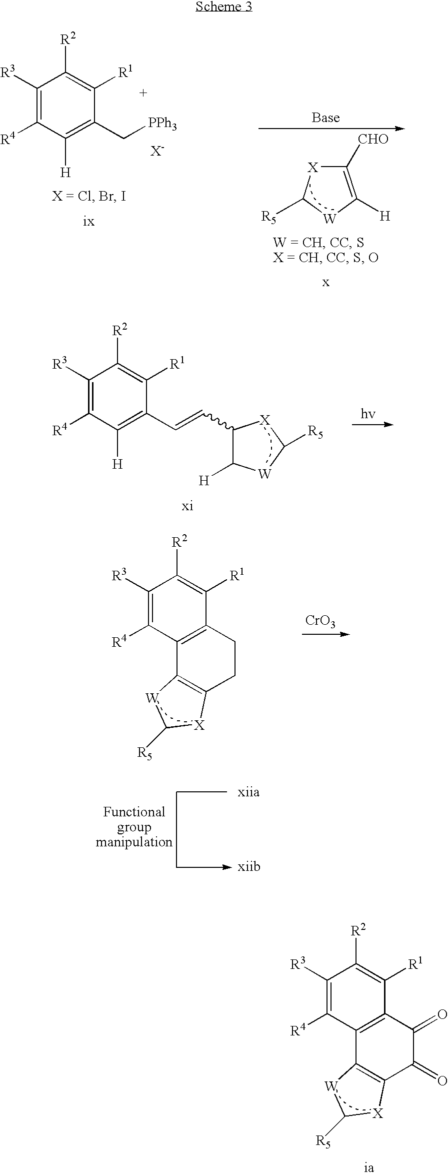

As exemplified in Scheme 4, heterophenanthrene xiia-1 could be elaborated to heterophenanthrene xiib-2 via a two-step process, by treatment with isopropenyl acetate in the presence of a catalyst such as palladium acetate and a suitable stannane such as tributyl tin methoxide and a suitable ligand such as tri-o-tolyl phosphine in a suitable solvent such as toluene to afford the functionalized heterophenanthrene xiib-1. Treatment of this heterophenanthrene xiib-1 with a pre-formed mixture of titanium tetrachloride and methyl lithium affords the tertiary alcohol xiib-2, which is oxidized with CrO₃ in acetic acid to produce heterophenanthrenequinone ia-1, which is further elaborated to the corresponding imidazoles Ia-d as described in Scheme 1.

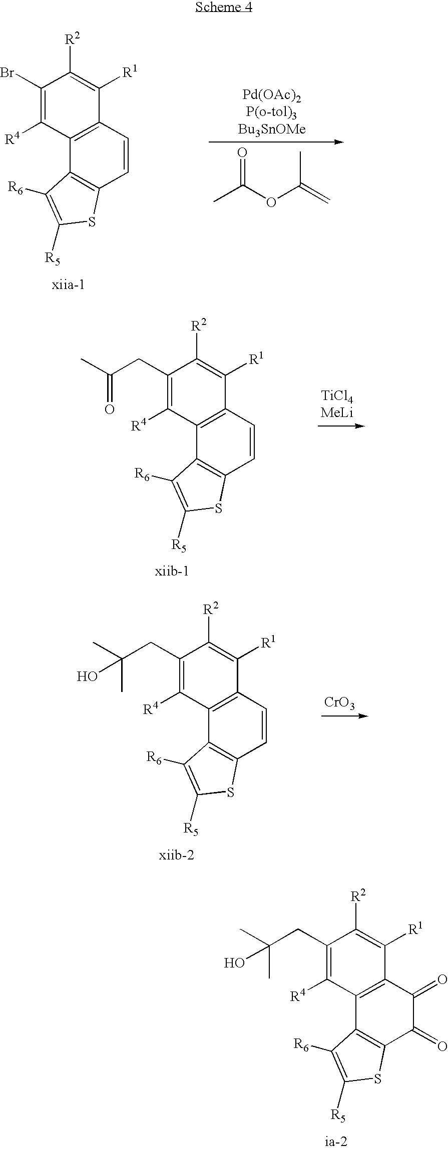

The methylene tertiary alcohol substituent present in xiib-2 could also be incorporated by the sequence of transformations shown in Scheme 5. Treatment of heterophenanthrene xiia-1 sequentially with methyl lithium and butyl lithium, followed by isobutylene oxide in the presence of boron trifluoroetherate in a solvent such as THF directly produces the heterophenanthrene xiib-2 functionalized with the methylene tertiary alcohol. Treatment of this alcohol with sodium hydride in THF, followed by t-butyldimethylsilylchloride affords the TBS-protected alcohol, which was oxidized with CrO₃ in acetic acid and subsequently converted to the heterophenanthrene imidazole Ia-1 by treatment with an appropriately substituted aldehyde ii and a reagent such as NH4OAc or NH4HCO3 in a solvent such as acetic acid. Deprotection of the tertiary alcohol substituent of imidazole Ia-1 with TBAF in THF, followed by treatment with CuCN in a solvent such as DMF or DMSO produces the mono or bis-nitrile (M=CCN) Ic-1.

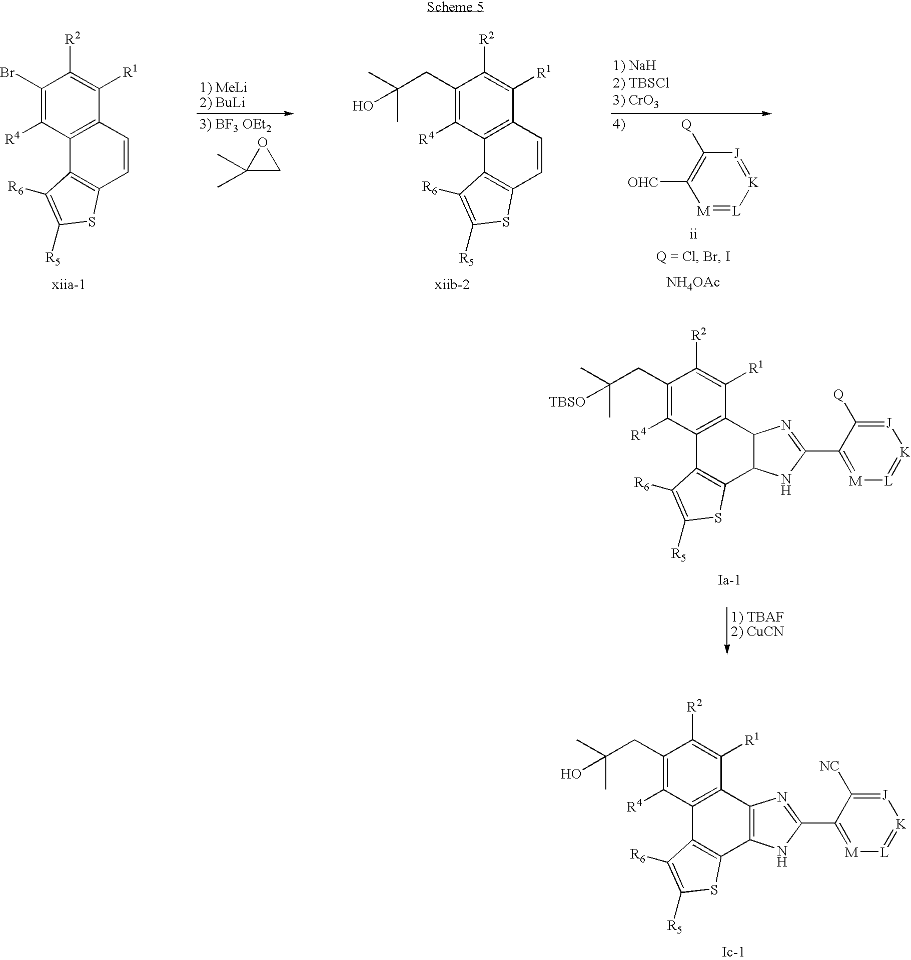

### 2) Pyridine Series

The compounds of Formula Ie-h of the present invention can be prepared according to the synthetic routes outlined in Schemes 6 to 11 below and by following the methods described therein. The imidazole of Formula Ie-f may be prepared in a multi-step sequence from the requisite azaphenanthrenequinone ib. The azaphenanthrene imidazole Ie is obtained by treating the azaphenanthrenequinone ib and an appropriately substituted aldehyde ii with a reagent such as NH₄OAc or NH₄HCO₃ in a solvent such as acetic acid. Imidazole Ie could be further elaborated to imidazole If (as exemplified in Scheme 10) by the appropriate transformation of any of the functional groups R₁ to R₆. Treatment of the imidazoles Ie or If with CuCN or NaCN in a solvent such as DMF or DMSO produces the mono or bis-nitrile (M=CCN) Ig. Subsequent functional group interconversion can be done at any of the R₁ to R₆ positions. For example, if one or more of the R₁ to R₆ substituents equal Cl, Br or I and if M is different from CBr or CI, Ig could be converted to Ih by placing Ig in the presence of a monosubstituted alkynyl, a stannane, a boronic acid, a borane or a boronate under conditions that promote cross coupling reactions, such as heating in the presence of a catalyst, such as Pd(PPh₃)₄ and CuI, in the presence of a base, such as sodium carbonate or diisopropylamine, and in a suitable solvent, such as THF, DMF or DME. This last exemplified step, or any other appropriate functional group transformation, can be iteratively repeated on R₁ to R₆.

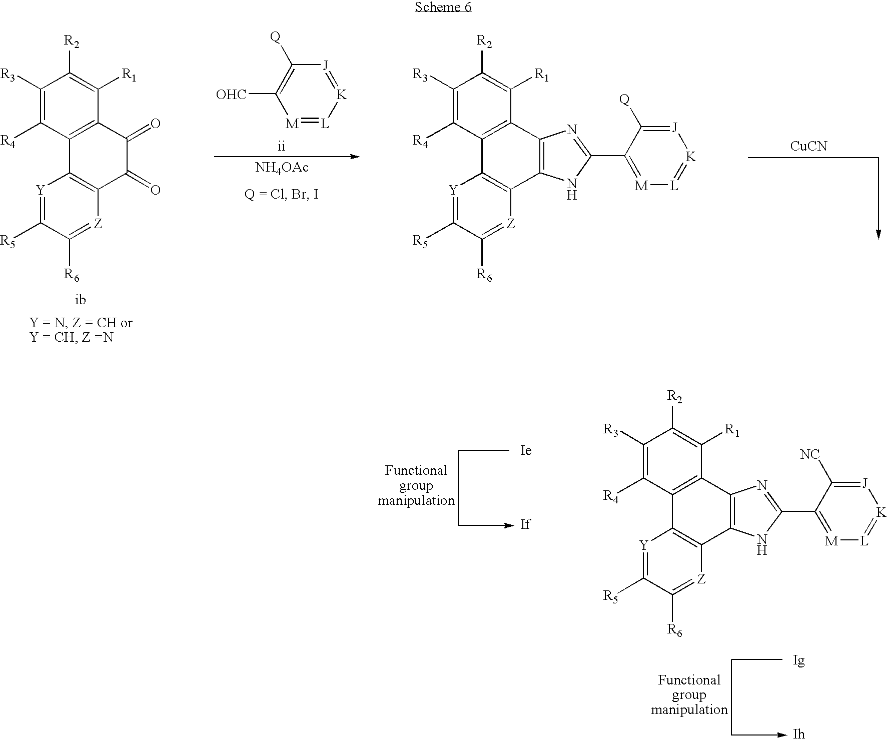

Azaphenanthrenequinone ib can be prepared according to the sequences outlined in Schemes 7-9. As shown in Scheme 7, commercially available azaphenanthrenes xiii-a can be directly oxidized to azaphenanthrenequinone ib by treatment with an oxidizing agent such as diiodine pentoxide in a solvent such as acetic acid. Alternatively, commercially available 7,8-benzoquinoline could be treated with an alkyl or aryl lithium reagent, such as phenyl lithium, in a solvent such as toluene, followed by oxidation with an oxidizing agent such as manganese dioxide in a solvent such as methylene chloride to produce the functionalized azaphenanthrene xiii-b, which is then oxidized to azaphenanthrenequinone ib as described above.

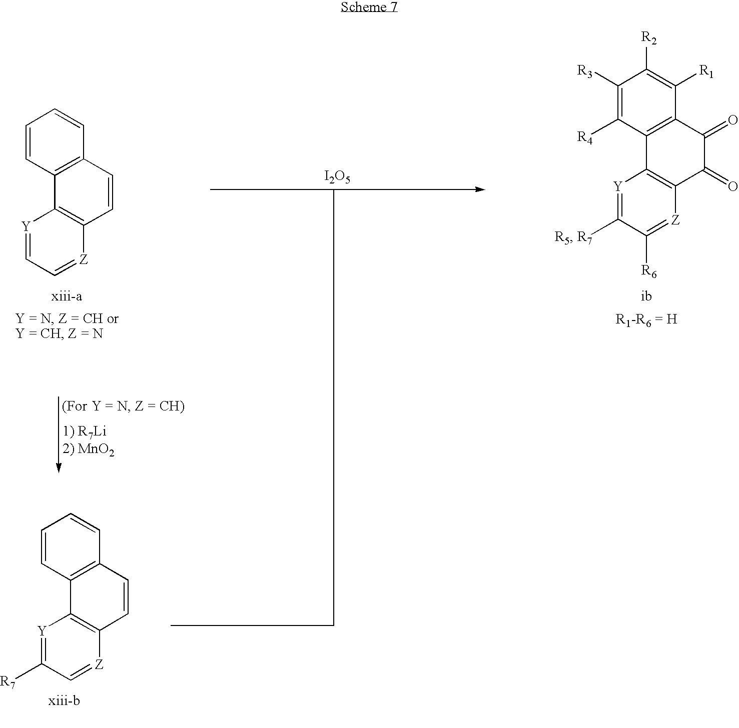

Azaphenanthrenequinone ib can also be prepared by the sequence shown in Scheme 8. Treatment of a 3-methyl-2-bromopyridine xiv with a benzamide boronic acid xv (prepared for example via ortho-lithiation of an aryl benzamide, followed by quenching with trimethylborate and subsequent acidic workup) in the presence of a catalyst such as Pd(PPh₃)₄ and a base such as sodium carbonate in a suitable solvent such as DME produces the bi-aryl xvia, which can be treated with a deprotonating agent such as LDA or a mixture of KHMDS and diisopropylamine in a solvent such as THF to produce the azaphenanthrol xviia. This azaphenanthrol xviia can be directly oxidized to azaphenanthrenequinone ib by treatment with a catalytic amount of N,N′-Bis(salicylidene)ethylenediaminocobalt (II) hydrate [Co(SALEN)₂] in a solvent such as DMF, in the presence of air. Alternatively, the bi-aryl xvia can undergo a series of functional group transformations, such as oxidation of the pyridine nitrogen with an oxidizing agent such as MCPBA in a solvent such as methylenechloride, followed by rearrangement with phosphorus oxychloride in a solvent such as DMF to afford a 2-chloropyridine derivative xvib. This derivatized bi-aryl xvib can undergo the same cyclisation and oxidation procedures as described above to afford azaphenanthrene ib. In a similar manner, the azaphenanthrol xviia can undergo a series of functional group transformations, for example, if one or more of the R₁ to R₇ substituents equal Cl, Br or I, xviia could be converted to xviib by placing xviia in the presence of a monosubstituted alkynyl, a stannane, a boronic acid, a borane, a boronate or carbon monoxide under conditions that promote cross coupling reaction, such as heating in the presence of a catalyst, such as Pd(PPh₃)₄ and CuI, in the presence of a base, such as sodium carbonate or diisopropylamine, and in a suitable solvent, such as THF, DMF or DME or in a mixture of an alcoholic solvent such as methanol and DMF.

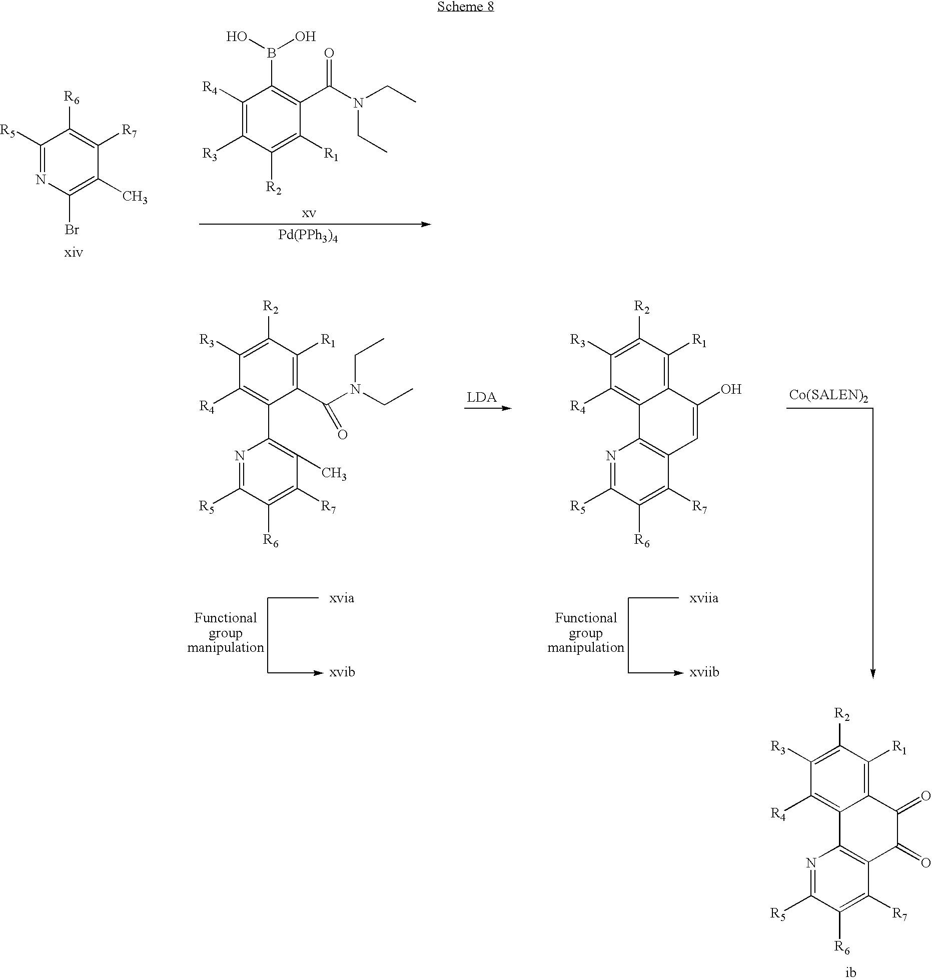

A third synthetic route to azaphenanthrenequinone ib is described in Scheme 9. Treatment of a 2-bromo-3-pyridinecarboxaldehyde xviii with a 3-alkoxyboronic acid xix in the presence of a catalyst such as Pd(PPh₃)₄ and a base such as sodium carbonate in a suitable solvent such as DME produces the bi-aryl xx. Treatment of this aldehyde xx with a solution of 2-trimethylsilyl-1,3-dithiane previously treated with butyllithium, followed by treatment with mercury (II) chloride in a mixture of methanol and water affords the ester xxia. This ester xxia can be hydrolyzed with a base such as sodium hydroxide in a suitable mixture of solvents such as THF and methanol to produce acid xxii. Alternatively, ester xxia can undergo a series of functional group transformations such as oxidation of the pyridine nitrogen with an oxidizing agent such as MCPBA in a solvent such as methylenechloride, followed by rearrangement with phosphorus oxychloride in a solvent such as DMF to afford a 2-chloropyridine derivative xxib, which can be hydrolyzed to acid xxii as described above. The acid xxii is acylated with N-(1-methanesulfonyl)benzothiazole (prepared from benzotriazole and methylsulfonyl chloride as described in JOC 2000, 65, 8210) in a solvent such as THF, in the presence of a base such as triethylamine to produce the N-acylbenzothiazole xxiii. Intramolecular cyclisation of xxiii is accomplished in the presence of a Lewis acid such as aluminum trichloride in a solvent such as 1,2-dichloroethane to produce the azaphenanthrol xviic which can be directly oxidized to azaphenanthrenequinone ib either by treatment with a catalytic amount of N,N′-Bis(salicylidene)ethylenediaminocobalt (II) hydrate [Co(SALEN)₂] in a solvent such as DMF, in the presence of air or with an oxidizing agent such as diiodine pentoxide in a solvent such as acetic acid. Alternatively, the azaphenanthrol xviic can undergo a series of functional group transformations (as exemplified in Scheme 11) to produce functionalized azaphenanthrol xviid, which undergoes oxidation to azaphenanthrenequinone ib as described above.

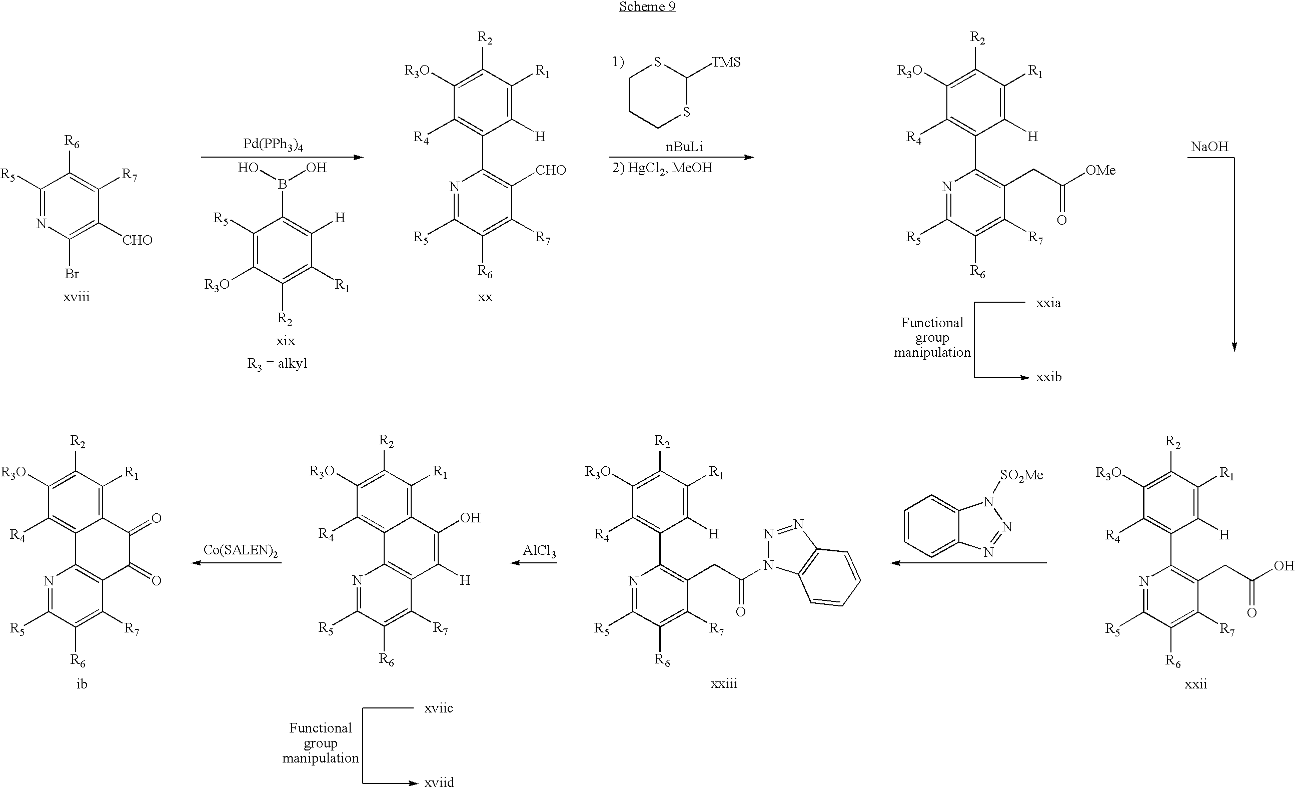

Some of the functional group transformations possible on the various intermediates in the above schemes are exemplified in Schemes 10 and 11. As shown in Scheme 10, imidazole Ie-1 could be treated with an oxidizing agent such as MCPBA in a solvent such as methylene chloride to produce the N-oxide If-1, which could be either treated with acetic anhydride to form the pyridone If-2 or treated with phosphorus oxychloride in a solvent such as DMF to produce the 2-chloropyridine derivative If-3. These imidazoles If-1, If-2 and If-3 could be treated with CuCN in a solvent such as DMF or DMSO to produce the mono or bis-nitrile (M=CCN) Ig as shown in Scheme 6.

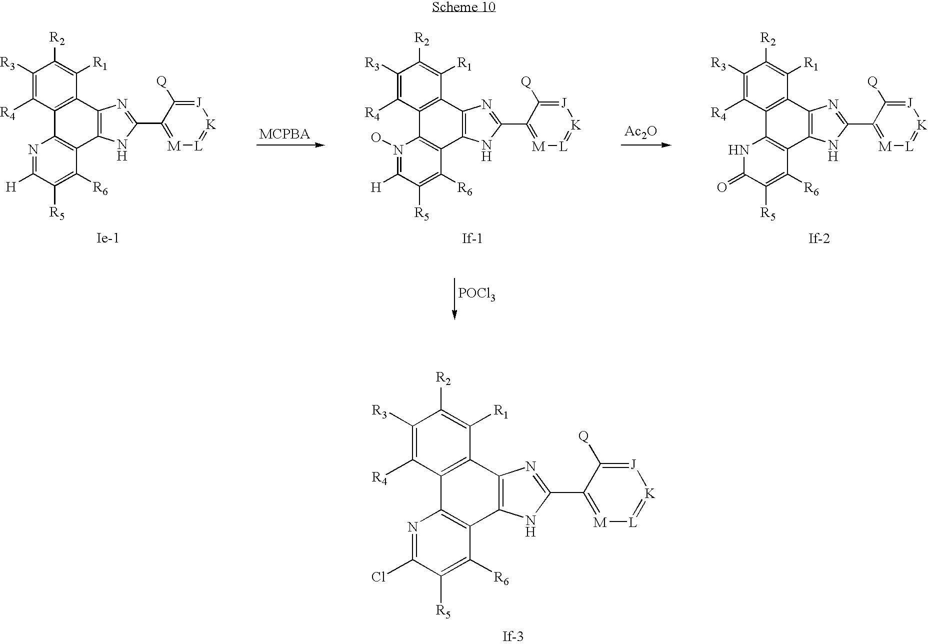

As shown in Scheme 11, protection of the haloazaphenanthrol xviic with an appropriate protecting group using a reagent such as t-butyldiphenylsilylchloride in the presence of a base, such as imidazole, in a suitable solvent such as DMF affords the protected haloazaphenanthrol xviid-1. The alkoxy substituent on this haloazaphenanthrol xviid-1 is then deprotected using an agent such as boron tribromide in a solvent such as methylene chloride to afford the phenol xviid-2 which can undergo a Mitsonobu reaction in the presence of a primary alcohol R₈OH, triphenyl phosphine and an activating agent such as di-t-butylazodicarboxylate in a suitable solvent such as THF to produce the haloazaphenanthrol xviid-3. This haloazaphenanthrol xviid-3 could then be elaborated to azaphenanthrene xviid-5 in a two-step process, by treatment with isopropenyl acetate in the presence of a catalyst such as palladium acetate and a suitable stannane such as tributyl tin methoxide and a suitable ligand such as tri-o-tolyl phosphine in a suitable solvent such as toluene to afford the functionalized azaphenanthrene xviid-4. Treatment of this azaphenanthrene xviid-4 with a pre-formed mixture of titanium tetrachloride and methyl lithium affords the tertiary alcohol xviid-5, which is treated with tetrabutylammonium fluoride in a solvent such as THF to liberate the phenol xviid-6. This azaphenanthrol xviid-6 is treated with either a catalytic amount of N,N′-Bis(salicylidene)ethylenediaminocobalt (II) hydrate [Co(SALEN)₂] in a solvent such as DMF in the presence of air or with an oxidizing agent such as diiodine pentoxide in a solvent such as acetic acid to produce the azaphenanthrenequinone ib-1, which is further elaborated to the corresponding imidazoles Ie-h as described in Scheme 6.

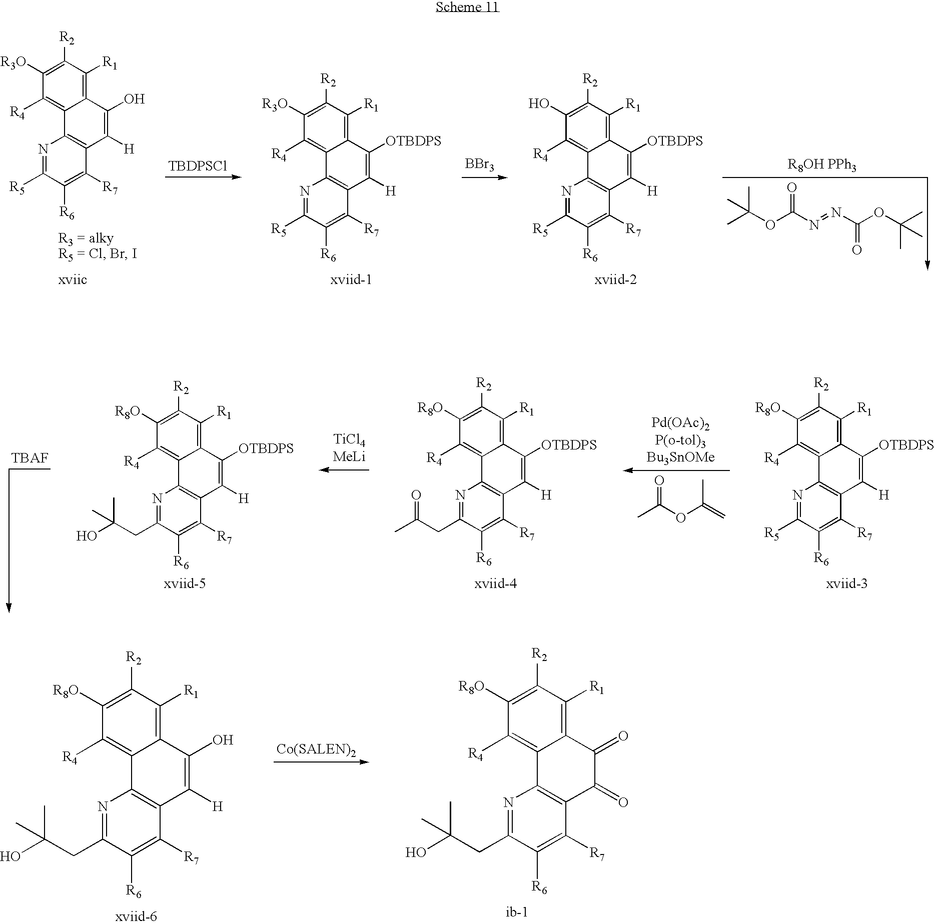

### 3) Indole Series

The compounds of Formula Ii-m of the present invention can be prepared according to the synthetic routes outlined in Schemes 12 to 13 below and by following the methods described therein. The imidazole of Formula Ii-j may be prepared in a multi-step sequence from the requisite heterophenanthrenequinone ic. The heterophenanthrene imidazole Ii is obtained by treating the heterophenanthrenequinone ic and an appropriately substituted aldehyde ii with a reagent such as NH₄OAc or NH₄HCO₃ in a solvent such as acetic acid. Imidazole Ii could be further elaborated to imidazole Ij by the appropriate transformation of any of the functional groups R₁ to R₈. Treatment of the imidazoles Ii or Ij with CuCN in a solvent such as DMF or DMSO produces the mono or bis-nitrile (M=CCN) Ik. Subsequent functional group interconversion can be done at any of the R₁ to R₈ positions. For example, if one or more of the R₁ to R₈ substituents equal Cl, Br or I and if M is different from CBr or CI, Ik could be converted to Im by placing Ik in the presence of a monosubstituted alkynyl, a stannane, a boronic acid, a borane or a boronate under conditions that promote cross coupling reactions, such as heating in the presence of a catalyst, such as Pd(PPh₃)₄ and CuI, in the presence of a base, such as sodium carbonate or diisopropylamine, and in a suitable solvent, such as THF, DMF or DME. This last exemplified step, or any other appropriate functional group transformation, can be iteratively repeated on R₁ to R₈.

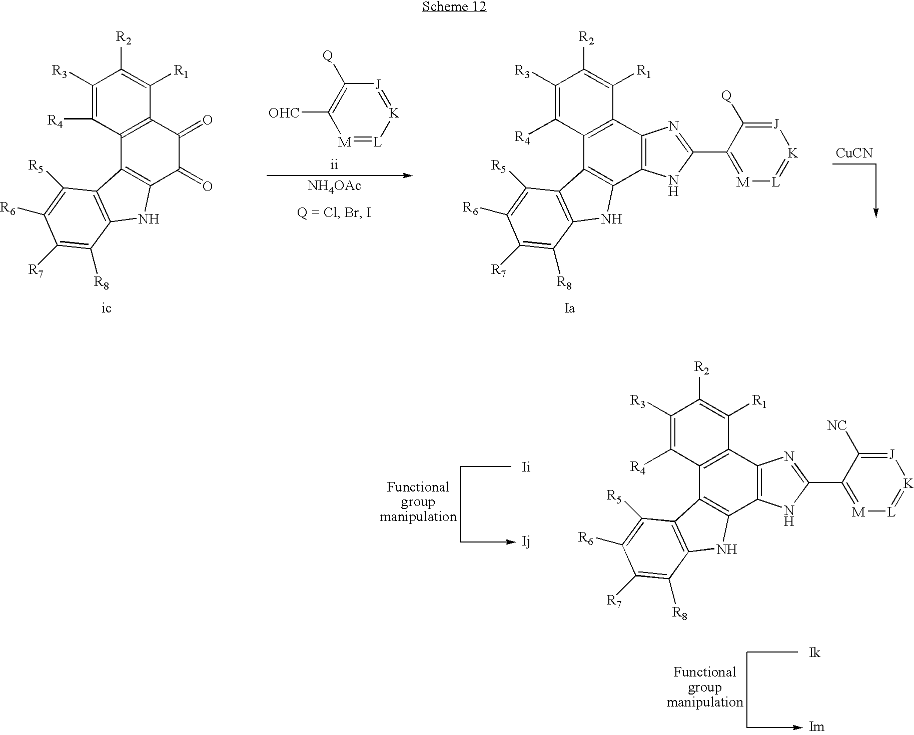

Heterophenanthrenequinone ic can be prepared according to the sequence outlined in Scheme 13. As shown in Scheme 13, treatment of an appropriately substituted bromo-phenylacetic ester iii (which could be prepared for example, by esterification of phenylacetic acids, displacement of activated aryl fluorides with malonate derivatives followed by decarboxylation, or Wolffe rearrangement of benzoic acids) with an indole xxiv in the presence of a catalyst such as Pd(OAc)₂ and in the presence of a ligand such as P(t-Bu)₃ and a base, such as potassium carbonate, in an suitable solvent, such as xylenes, followed by hydrolysis of the ester functionality with a base such as sodium hydroxide in a suitable mixture of solvents such as THF and methanol produces the phenylacetic acid xxv. Conversion of the acid xxv into its acyl chloride upon treatment with a suitable reagent such as oxalyl chloride in the presence of a catalytic amount of DMF in a solvent such as THF, followed by intramolecular cyclisation in the presence of a Lewis acid such as aluminum trichloride in a solvent such as 1,2-dichloroethane produces the heterophenanthrol xxvi. This phenanthrol xxvi can be directly oxidized to heterophenanthrenequinone ic by treatment with a catalytic amount of N,N′-Bis(salicylidene)ethylenediaminocobalt (II) hydrate [Co(SALEN)₂] in a solvent such as DMF, in the presence of air.

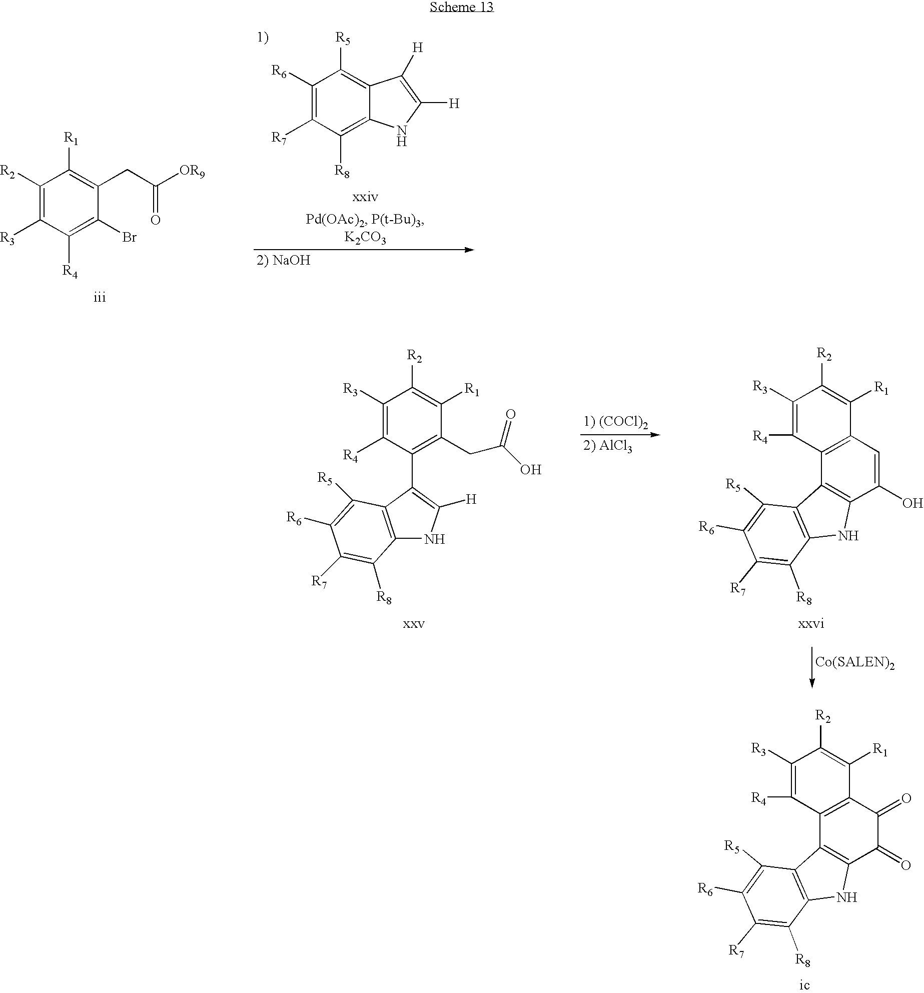

### 4) Bis-Thiophene Series

The compounds of Formula In-q of the present invention can be prepared according to the synthetic routes outlined in Schemes 14 to 16 below and by following the methods described therein. The imidazole of Formula In-o may be prepared in a multi-step sequence from the requisite heterophenanthrenequinone id. The heterophenanthrene imidazole In is obtained by treating the heterophenanthrenequinone id and an appropriately substituted aldehyde ii with a reagent such as NH₄OAc or NH₄HCO₃ in a solvent such as acetic acid. Imidazole In could be further elaborated to imidazole Io by the appropriate interconversion of any of the functional groups R₁ to R₄, such as halogenation with a suitable halogenating agent such as bromine or N-bromosuccinimide, in a suitable solvent such as THF or acetic acid, or transmetallation with an organometallic reagent, such as butyl lithium, in a suitable solvent such as THF, followed by the addition of an electrophile, such as iodine or carbon dioxide, or any other appropriate functional group interconversion. Treatment of the imidazoles In or Io with CuCN in a solvent such as DMF or DMSO produces the mono or bis-nitrile (M=CCN) Ip. Subsequent functional group interconversion can be done at any of the R₁ to R₄ positions (as exemplified in Scheme 16). For example, if one or more of the R₁ to R₈ substituents equal Cl, Br or I and if M is different from CBr or CI, Ip could be converted to Iq by placing Ip in the presence of a monosubstituted alkynyl, a stannane, a boronic acid, a borane or a boronate under conditions that promote cross coupling reactions, such as heating in the presence of a catalyst, such as Pd(PPh₃)₄ and CuI, in the presence of a base, such as sodium carbonate or diisopropylamine, and in a suitable solvent, such as THF, DMF or DME. This last exemplified step, or any other appropriate functional group transformation, can be iteratively repeated on R₁ to R₄.

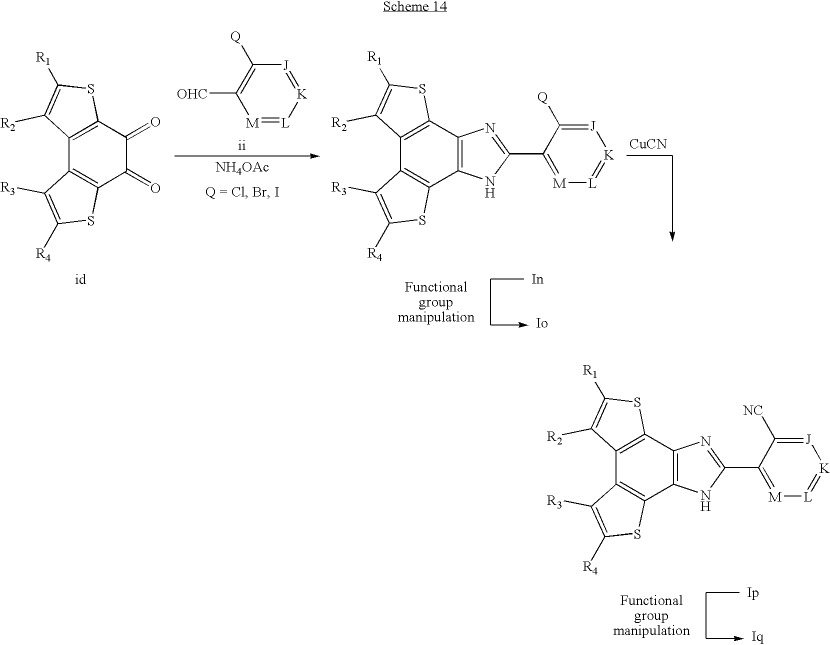

Heterophenanthrenequinone id can be prepared according to the sequence outlined in Scheme 15. As shown in Scheme 15, treatment of an appropriately substituted bromo-thiophene xxvii with a thiophene boronic acid xxviii in the presence of a catalyst such as Pd(PPh₃)₄ and in the presence of a base, such as sodium carbonate, in an suitable solvent, such as DME produces the bis-thiophene xxix. Treatment of this bis-thiophene xxix with oxalyl chloride in a solvent such as 1,2-dichloroethane produces the heterophenanthrenequinone id.

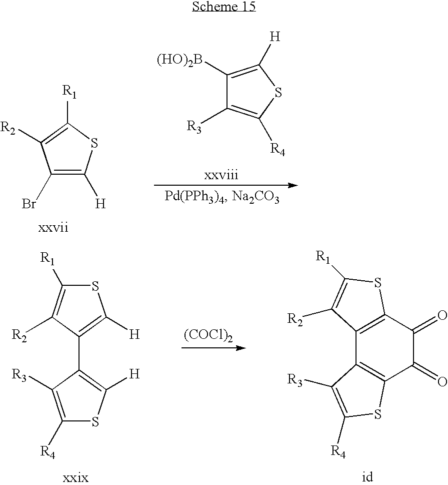

As exemplified in Scheme 16, treatment of imidazole Ip-1 with NBS in a suitable mixture of solvents such as THF and water produces the di-bromo imidazole Iq-1.

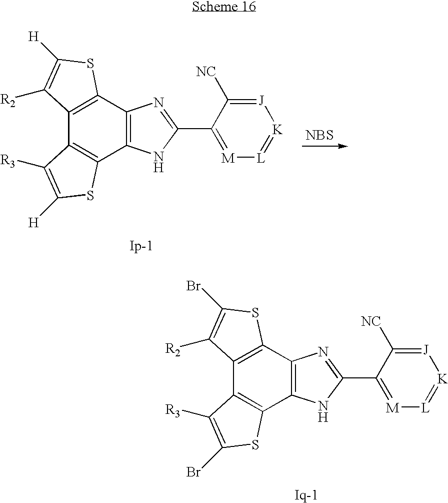

### EXAMPLES

The invention is exemplified by the following non-limiting examples:

TABLE 1

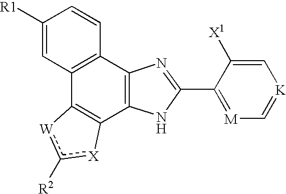

| EX | W | X | R¹ | R² | X¹ | M | K |
|---|---|---|---|---|---|---|---|
| 1 | CH | S | H | H | Cl | CF | CH |
| 2 | S | CH | H | Cl | Cl | CF | CH |
| 3 | CH | O | H | H | Cl | CF | CH |
| 4 | S | CH | H | H | Cl | CF | CH |
| 5 | CH | S | CN | H | Cl | CF | CH |
| 6 | CH | S | Cl | H | Cl | CF | CH |
| 7 | CH | S | Cl | H | CN | CF | CH |
| 8 | CH | S | Cl | H | Br | CBr | CH |
| 9 | CH | S | Cl | H | CN | CCN | CH |
| 10 | CH | S | Cl | H | Br | CBr | N |
| 11 | CH | S | Cl | H | CN | CCN | N |
| 12 | CH | O | Cl | H | CN | CCN | CH |
| 13 | CH | O | Cl | H | Br | CBr | N |
| 14 | CH | O | Cl | H | CN | CCN | N |
| 15 | CH | S | Cl | Br | Cl | CF | CH |
| 16 | S | CH | H | Br | Cl | CF | CH |
| 17 | CH | S | Cl | Br | CN | CCN | CH |
| 18 | CH | S | Cl | 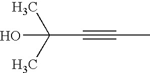 | CN | CCN | CH |
| 19 | CBr | S | Cl | Br | Br | CBr | CH |
| 20 | CH | S | Cl | Cl | CN | CCN | CH |
| 21 | CH | S | Br | Cl | Br | CBr | CH |
| 22 | CH | S | Br | Cl | CN | CCN | CH |
| 23 | CH | S | 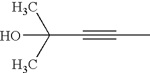 | Cl | CN | CCN | CH |
| 24 | CBr | S | Cl | Cl | Br | CBr | CH |
| 25 | CBr | S | Cl | Cl | CN | CCN | CH |
| 26 | CCl | S | Br | Cl | Br | CBr | CH |
| 27 | CCl | S | Br | Cl | CN | CCN | CH |
| 28 | CCl | S | 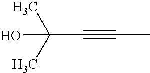 | Cl | CN | CCN | CH |
| 29 | CCl | S | 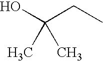 | Cl | CN | CCN | CH |
| 30 | CH | S | 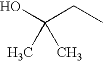 | Cl | CN | CCN | CH |
| 31 | CH | S | 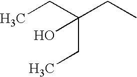 | Cl | CN | CCN | CH |

TABLE 2

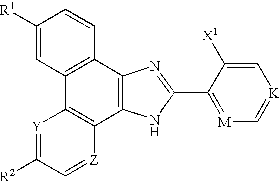

| Ex | Y | Z | R¹ | R² | X¹ | M | K |
|---|---|---|---|---|---|---|---|
| 32 | CH | N | H | H | Cl | CF | CH |
| 33 | N | CH | H | H | Cl | CF | CH |
| 34 | NO | CH | H | H | Cl | CF | CH |
| 35 | NH | CH | H | O | Cl | CF | CH |
| 36 | N | CH | H | H | Br | CBr | CH |
| 37 | N | CH | H | H | CN | CCN | CH |
| 38 | NO | CH | H | H | Br | CBr | CH |
| 39 | N | CH | H | Cl | CN | CCN | CH |
| 4O | N | CH | H | Ph | Br | CBr | CH |
| 41 | N | CH | H | Ph | CN | CCN | CH |
| 42 | N | CH | OCH3 | Cl | Br | CBr | CH |
| 43 | N | CH | OCH3 | Cl | CN | CCN | CH |
| 44 | N | CH | 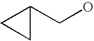 | 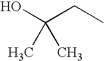 | Br | CBr | CH |
| 45 | N | CH | 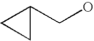 | 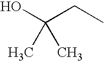 | CN | CCN | CH |
| 46 | N | CH | 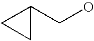 | Cl | CN | CCN | CH |
| 47 | N | CH | 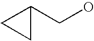 | 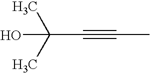 | CN | CCN | CH |
| 48 | N | CH | 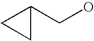 | 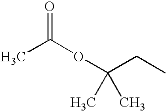 | CN | CCN | CH |
| 49 | N | CH | 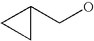 | CN | CN | CCN | CH |
| 50 | N | CH | 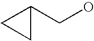 | 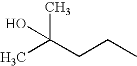 | CN | CCN | CH |
| 51 | N | CH | 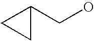 | Cl | CN | CCN | CF |
| 52 | N | CH | 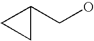 | 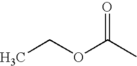 | CN | CCN | CH |
| 53 | N | CH | 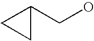 | 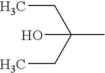 | CN | CCN | CH |
| 54 | N | CH | 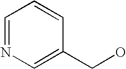 | Cl | CN | CCN | CH |
| 55 | N | CH | 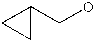 | 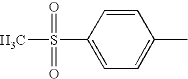 | CN | CCN | CH |

TABLE 3

| Ex | Structure |
|---|---|
| 56 | 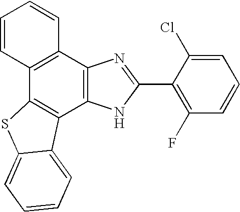 |
| 57 | 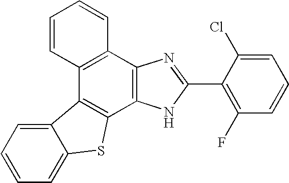 |
| 58 | 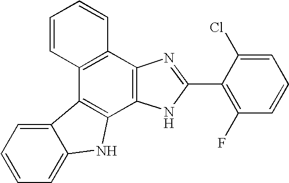 |
| 59 | 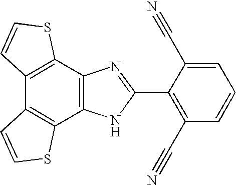 |
| 60 | 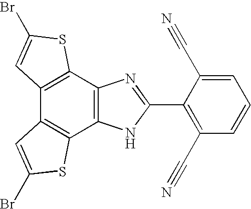 |

### Example 4

### 2-(2-chloro-6-fluorophenyl)-3H-thieno[3′,2′:3,4]naphtho[1,2-d]imidazole

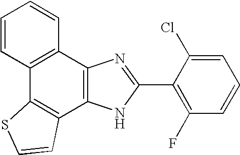

### Step 1: Methyl (2-bromophenyl)acetate

To a solution of (2-bromophenyl)acetic acid (14.5 g, 67.4 mmol) in methanol (300 mL) at 0° C. was slowly added thionyl chloride (7.4 mL, 1101 mmol). The resulting solution was stirred at 0° C. for 15 min, followed by room temperature overnight. The reaction was concentrated to yield methyl (2-bromophenyl)acetate (16.4 g).

### Step 2: Methyl [2-(4,4,5,5-tetramethyl-1,3,2-dioxaborolan-2-yl)phenyl]acetate

A mixture of methyl (2-bromophenyl)acetate (4.58 g, 20 mmol) from Step 1 above, diboron pinacol ester (7 g, 27 mmol), Pd(dppf)Cl₂.CH₂Cl₂ (653 mg, 0.8 mmol) and potassium acetate (6.87 g, 70 mmol) in DMF (130 mL) was purged under nitrogen atmosphere for 10 min, followed by heating at 80° C. overnight. Further equivalents of palladium catalyst (300 mg) were added and heating continued for 5 h, after which the reaction mixture was diluted with water. The aqueous layer was extracted with diethyl ether, the organic layer washed with water (3 times), dried over Na2SO4, filtered and concentrated. The material was purified by flash chromatography on silica (2.5-10% ethyl acetate in toluene) to provide methyl [2-(4,4,5,5-tetramethyl-1,3,2-dioxaborolan-2-yl)phenyl]acetate (1.72 g) as a colourless syrup.

### Step 3: Methyl [2-(2-thienyl)phenyl]acetate

A mixture of methyl [2-(4,4,5,5-tetramethyl-1,3,2-dioxaborolan-2-yl)phenyl]acetate (345 mg, 1.25 mmol) from Step 2 above, 2-bromothiophene (242 μL, 2.5 mmol), Pd(dppf)Cl₂ (51 mg, 0.06 mmol) and sodium carbonate (2 M, 3 mL) in DMF (12 mL) was purged with nitrogen for 10 min, followed by heating at 85° C. for 2 h. The reaction mixture was then cooled to room temperature, diluted with water and extracted with diethyl ether. The organic layer was washed with water (3 times), dried over Na2SO4, filtered and concentrated. The material was purified by flash chromatography on silica (10% ethyl acetate in hexanes) to provide methyl [2-(2-thienyl)phenyl]acetate (148 mg) as a colourless syrup.

### Step 4: [2-(2-thienyl)phenyl]acetic acid

To a solution of methyl [2-(2-thienyl)phenyl]acetate (181 mg, 0.78 mmol) from Step 3 above in THF (6 mL)/methanol (6 mL), was added sodium hydroxide (1N, 3 mL). The mixture was stirred at room temperature for 1.5 h, then at 45° C. for 30 min, after which it was cooled to room temperature and concentrated to a small volume. The residue was diluted with water and acidified with HCl (1 N) to yield a precipitate which was filtered to afford [2-(2-thienyl)phenyl]acetic acid (126 mg) as a white solid.

### Step 5: Naphtho[1,2-b]thiophene-4-ol

To a solution of [2-(2-thienyl)phenyl]acetic acid (118 mg, 0.54 mmol) from Step 4 above in THF (4 mL) at 0° C. was added oxalyl chloride (137 mg, 108 mmol), followed by DMF (20 μL). The resulting yellow solution was stirred at 0° C. for 2 h and then concentrated at reduced pressure (at 30° C.). The residue was co-evaporated with 1,2-dichloroethane (4 mL) to provide a yellow syrup which was dissolved in 1,2-dichloroethane (4 mL). To this solution was added aluminium trichloride (108 mg, 0.81 mmol) at room temperature. The mixture was stirred at room temperature for 1 h, then diluted with CH₂Cl₂ and quenched with cold water. The aqueous layer was extracted with CH₂Cl₂, the organic layer dried over Na2SO4, filtered and concentrated. The material was purified by flash chromatography on silica (25% ethyl acetate in hexanes) to provide naphtho[1,2-b]thiophene-4-ol (103 mg) as a beige solid.

### Step 6: Naphtho[1,2-b]thiophene-4,5-dione

A mixture of naphtho[1,2-b]thiophene-4-ol (94 mg, 0.47 mmol) from Step 5 above and N,N′-Bis(salicylidene)ethylenediaminocobalt (II) hydrate (16 mg, 0.05 mmol) in DMF (6 mL) was stirred at room temperature overnight, with air bubbled through the reaction mixture. The mixture was then quenched with water, stirred for 5 min and the resulting solid precipitate filtered and washed with water to afford naphtho[1,2-b]thiophene-4,5-dione (61 mg) as a brick-red solid.

### Step 7: 2-(2-chloro-6-fluorophenyl)-3H-thieno[3′,2′:3,4]naphtho[1,2-d]imidazole

To a solution of naphtho[1,2-b]thiophene-4,5-dione (54 mg, 0.252 mmol) from Step 6 above, in acetic acid (5 mL) was added ammonium acetate (195 mg, 2.52 mmol) and 2-chloro-6-fluorobenzaldehyde (52 mg, 0.33 mmol). The mixture was heated at reflux for 6 h, then poured into water, stirred for 5 minutes and filtered. The solid was purified by flash chromatography on silica (30% ethyl acetate in hexanes) to provide 2-(2-chloro-6-fluorophenyl)-3H-thieno[3′, 2′:3,4]naphtho[1,2-d]imidazole (75 mg) as a tan solid. ¹H NMR δ (ppm) (400 MHz, Acetone-d₆, mixture of tautomers): 12.8 (0.5 Ha, bs), 12.6 (0.5 Hb, bs), 8.73-8.71 (0.5 Ha, m), 8.46-8.41 (0.5 Hb, m), 8.30-8.25 (0.5 Ha, 0.5 Hb, m), 7.98-7.87 (2 Ha, b, m), 7.71-7.62 (3 Ha, b, m), 7.54 (1 Ha, b, d), 7.40 (1 Ha, b, t)

### Example 9

### 2-(8-chloro-3H-thieno[2′,3′:3,4]naphtho[1,2-d]imidazol-2-yl)isophthalonitrile

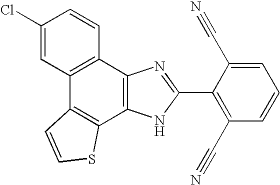

### Step 1: Benzyl (2-bromo-4-chlorophenyl)acetate

To a solution of 2-bromo-4-chlorobenzoic acid (1.18 g, 5 mmol) in THF (40 ml) at 0° C., was added oxalyl chloride (1.27 g, 10 mmol) followed by DMF (20 μL). The reaction mixture was stirred at 0° C. for 1 h and then concentrated at reduced pressure (at 30° C.). The residue was co-evaporated with acetonitrile (4 mL) and the residue dried under high vacuum. To a solution of this crude acid chloride in THF/CH3CN (1:1, 20 mL) at 0° C., was added (trimethylsilyl)diazomethane (2 M in hexanes, 3.13 mL), followed by triethylamine (870 μL, 6.25 mmol). The resulting yellow solution was stirred at 0° C. for 1 h and at 5° C. overnight. The reaction mixture was concentrated under reduced pressure to a semi-solid residue which was taken up in 2,4,6-trimethylpyridine (4 mL) and benzylalcohol (4 mL). The mixture was heated at 175° C. for 20 minutes, then cooled to room temperature and diluted with ethyl acetate. The organic layer was washed successively with 1 N HCl, water (2 times), dried over Na2SO4, filtered and concentrated. The material was purified by flash chromatography on silica (10% ethyl acetate in hexanes) to provide benzyl (2-bromo-4-chlorophenyl)acetate (926 mg, 55%) as a red-brown oil.

### Step 2: Benzyl [4-chloro-2-(3-thienyl)phenyl]acetate

A mixture of benzyl (2-bromo-4-chlorophenyl)acetate (424 mg, 1.25 mmol) from Step 1 above, 3-thienylboronic acid (320 mg, 2.5 mmol), cesium fluoride (570 mg, 3.75 mmol) and Pd (PPh₃)₄ (72 mg, 0.06 mmol) in DME (10 mL) was purged with nitrogen for 10 min, followed by heating at reflux for 3.5 h. The reaction mixture was then cooled to room temperature, diluted with water and ethyl acetate. The organic layer was washed with water (3 times), dried over Na2SO4, filtered and concentrated. The material was purified by flash chromatography on silica (100% toluene) to provide benzyl [4-chloro-2-(3-thienyl)phenyl]acetate (358 mg, 83%) as a yellow syrup.

### Step 3: [4-chloro-2-(3-thienyl)phenyl]acetic acid

This acid was prepared as described in Step 4 of Example 4, substituting benzyl [4-chloro-2-(3-thienyl)phenyl]acetate from Step 2 above for methyl [2-(2-thienyl)phenyl]acetate.

### Step 4: 8-chloronaphtho[2,1-b]thiophene-4-ol

This compound was prepared as described in Step 5 of Example 4, substituting [4-chloro-2-(3-thienyl)phenyl]acetic acid from Step 3 above for [2-(2-thienyl)phenyl]acetic acid.

### Step 5: 8-chloronaphtho[2,1-b]thiophene-4,5-dione

This dione was prepared as described in Step 6 of Example 4, substituting 8-chloronaphtho[2,1-b]thiophene-4-ol from Step 4 above for naphtho[1,2-b]thiophene-4-ol.

### Step 6: 8-chloro-2-(2,6-dibromophenyl)-3H-thieno[2′,3′:3,4]naphtho[1,2-d]imidazole

This imidazole was prepared as described in Step 7 of Example 4, substituting 8-chloronaphtho[2,1-b]thiophene-4,5-dione from Step 5 above for naphtho[1,2-b]thiophene-4,5-dione and substituting 2,6-dibromobenzaldehyde for 2-fluoro-6-chlorobenzaldehyde.

### Step 7: 2-(8-chloro-3H-thieno[2′,3′:3,4]naphtho[1,2-d]imidazol-2-yl)isophthalonitrile

To a DMF (5 mL) solution of 8-chloro-2-(2,6-dibromophenyl)-3H-thieno[2′,3′:3,4]naphtha[1,2-d]imidazole (212 mg, 0.43 mmol) from Step 6 above, was added CuCN (154 mg, 1.72 mmol). The reaction was stirred at 100° C. for 2 h, then at 115° C. for 20 minutes. Additional CuCN (80 mg) was added and the reaction mixture stirred at 115° C. for 1 h. It was cooled down to room temperature, diluted with water, ethyl acetate and 15% ammonium hydroxide. The mixture was stirred at room temperature for 20 minutes. The aqueous layer was extracted with ethyl acetate and the combined organic layers were washed with water (4 times), dried over Na2SO4, filtered and volatiles were removed under reduced pressure. The material was purified by flash chromatography on silica gel (66% ethyl acetate in hexanes) to afford 2-(8-chloro-3H-thieno[2′,3′:3,4]naphtho[1,2-d]imidazol-2-yl)isophthalonitrile (107 mg) as a yellow solid. ¹H NMR δ (ppm) (400 MHz, Acetone-d₆): 13.2 (1H, bs), 8.70-8.55 (2H, m), 8.40-8.28 (3H, m), 8.05 (1H, t), 7.85 (1H, d), 7.70 (1H, dd).

### Example 24

### 6-bromo-5,8-dichloro-2-(2,6-dibromophenyl)-3H-thieno[2′,3′:3,4]naphtho[1,2-d]imidazole

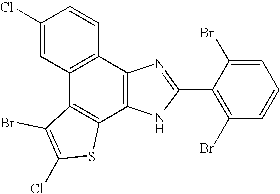

### Step 1: 4-bromo-2-[2-(4-chlorophenyl)vinyl]thiophene

To a solution of (4-chlorobenzyl)triphenylphosphonium chloride (17.3 g) in DMF (200 mL) at 0° C., was added NaH (60% in oil, 2 g). The mixture was stirred at 0° C. for 30 minutes, after which a solution of 4-bromothiophene-2-carbaldehyde (8 g) in DMF (20 mL) was added. The mixture was stirred at 0° C. for 30 minutes, then warmed up to room temperature and stirred 1 hr. The reaction mixture was quenched by the addition of 1 N HCl and Et2O. The aqueous layer was extracted with Et2O, the combined organic layers were washed with brine, dried over Na2SO4 and filtered. The volatiles were removed under reduced pressure and the residue was purified by flash chromatography on silica (100% hexanes) to afford 4-bromo-2-[2-(4-chlorophenyl)vinyl]thiophene (10 g) as a mixture of isomers.

### Step 2: 1-bromo-8-chloronaphtho[2,1-b]thiophene

To a 2 L vessel equipped with a pyrex inner water-cooled jacket was added 4-bromo-2-[2-(4-chlorophenyl)vinyl]thiophene (5 g) from Step 1 above in cyclohexane, followed by the addition of a solution of iodine (424 mg) in THF. The stirred solution was exposed to UV light for 24 hrs by inserting a 450 W medium pressure mercury lamp in the inner insert, while air was bubbled through the reaction mixture. The reaction was quenched with 10% Na2S2O3 and the aqueous layer was extracted with ethyl acetate. The combined organic layers were washed with brine, dried over Na2SO4, filtered and volatiles were removed under reduced pressure. The residue was purified by flash chromatography on silica (100% hexanes) to afford 1-bromo-8-chloronaphtho[2,1-b]thiophene (3.8 g).

### Step 3: 1-bromo-2,8-dichloronaphtho[2,1-b]thiophene

To a solution of 1-bromo-8-chloronaphtho[2,1-b]thiophene (306 mg) from Step 2 above in THF (30 ml)/water (3 mL), was added N-chlorosuccinimide (411 mg). The reaction mixture was heated at 70° C. overnight, then quenched with 10% Na2SO3 and ethyl acetate. The aqueous layer was extracted with ethyl acetate. The combined organic layers were washed with brine, dried over Na2SO4, filtered and volatiles were removed under reduced pressure. The residue was purified by flash chromatography on silica (100% hexanes) to afford 1-bromo-2,8-dichloronaphtho[2,1-b]thiophene (335 mg).

### Step 4: 1-bromo-2,8-dichloronaphtho[2,1-b]thiophene-4,5-dione

To a solution of 1-bromo-2,8-dichloronaphtho[2,1-b]thiophene (335 mg) from Step 3 above in acetic acid, was added CrO₃ (404 mg). The mixture was stirred at 120° C. for 2 h, cooled down to room temperature, poured into water and ethyl acetate. The aqueous layer was extracted with ethyl acetate. The combined organic layers were washed with brine, dried over Na₂SO₄, filtered and volatiles were removed under reduced pressure. The residue was purified by flash chromatography on silica (50% ethyl acetate in hexanes) to afford 1-bromo-2,8-dichloronaphtho[2,1-b]thiophene-4,5-dione (180 mg).

### Step 5: 6-bromo-5,8-dichloro-2-(2,6-dibromophenyl)-3H-thieno[2′,3′:3,4]naphtho[1,2-d]imidazole

To a solution of 1-bromo-2,8-dichloronaphtho[2,1-b]thiophene-4,5-dione (180 mg) from Step 4 above in acetic acid, was added ammonium acetate (766 mg) and 2,6-dibromobenzaldehyde (197 mg). The mixture was stirred at 120° C. overnight, cooled down to room temperature and quenched with water and ethyl acetate. The aqueous layer was extracted with ethyl acetate. The combined organic layers were washed with brine, dried over Na₂SO₄, filtered and volatiles were removed under reduced pressure. The residue was purified by flash chromatography on silica (25-50% ethyl acetate in hexanes) to afford 6-bromo-5,8-dichloro-2-(2,6-dibromophenyl)-3H-thieno[2′,3′:3,4]naphtho[1,2-d]imidazole (180 mg). ¹H NMR δ (ppm) (400 Mhz, Acetone-d₆): 9.74 (1H, s), 8.48 (1H, d), 7.87 (2H, d), 7.73 (1H, d), 7.49 (1H, t)

### Example 25

### 2-(6-bromo-5,8-dichloro-3H-thieno[2′,3′:3,4]naphtho[1,2-d]imidazol-2-yl)isophthalonitrile

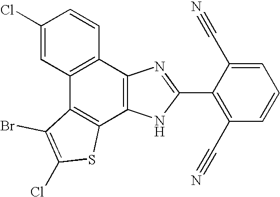

To a solution of 6-bromo-5,8-dichloro-2-(2,6-dibromophenyl)-3H-thieno[2′,3′:3,4]naphtho[1,2-d]imidazole (180 mg) from Example 24 in DMSO, was added CuCN (106 mg). The reaction mixture was stirred at 90° C. overnight, then cooled to room temperature and quenched with water and ethyl acetate. The aqueous layer was extracted with ethyl acetate. The combined organic layers were washed with brine, dried over Na₂SO₄, filtered and volatiles were removed under reduced pressure. The residue was purified by flash chromatography on silica (50-75% ethyl acetate in hexanes) to afford 2-(6-bromo-5,8-dichloro-3H-thieno[2′,3′:3,4]naphtho[1,2-d]imidazol-2-yl)isophthalonitrile (78 mg). ¹H NMR δ (ppm) (400 MhHz, Acetone-d₆): 9.86 (1H, s), 8.64 (1H, d), 8.40 (2H, d), 8.07 (1H, t), 7.83 (1H, d).

### Example 30

### 2-[5-chloro-8-(2-hydroxy-2-methylpropyl)-3H-thieno[2′,3′:3,4]naphtho[1,2-d]imidazol-2-yl]isophthalonitrile

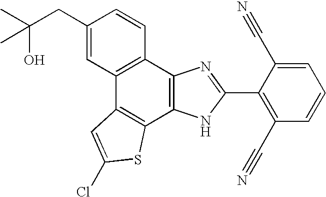

This imidazole could be prepared by two routes, as described below:

### Route A:

### Step 1: 2-[2-(4-bromophenyl)vinyl]-5-chlorothiophene

This compound was prepared as described in Step 1 of Example 24, substituting (4-bromobenzyl)triphenylphosphonium bromide for (4-chlorobenzyl)triphenylphosphonium chloride and substituting 5-chlorothiophene-2-carbaldehyde for 4-bromothiophene-2-carbaldehyde.

### Step 2: 8-bromo-2-chloronaphtho[2,1-b]thiophene

This naphthothiophene was prepared as described in Step 2 of Example 24, substituting 2-[2-(4-bromophenyl)vinyl]-5-chlorothiophene from Step 1 above for 4-bromo-2-[2-(4-chlorophenyl)vinyl]thiophene and irradiating the reaction mixture with UV light for 2 days instead of 24 h.

### Step 3: 1-(2-chloronaphtho[2,1-b]thien-8-yl)acetone

To a solution of 8-bromo-2-chloronaphtho[2,1-b]thiophene (3.67 g, 12.3 mmol) from Step 2 above in toluene (100 mL), was added isopropenylacetate (2.04 mL), followed by tributyl(methoxy)stannane (5.33 mL), palladium (II) acetate (277 mg) and tri-o-tolylphosphine (826 mg). The resulting mixture was heated at 85° C. overnight, cooled to room temperature and quenched with sodium bicarbonate and ethyl acetate. The aqueous layer was extracted with ethyl acetate, the organic layer washed with brine, dried over Na2SO4, filtered and concentrated. The crude material was purified by flash chromatography on silica (25% ethyl acetate in hexanes) to provide 1-(2-chloronaphtho[2,1-b]thien-8-yl)acetone (2 g, 39%).

### Step 4: 1-(2-chloronaphtho[2,1-b]thien-8-yl)-2-methylpropan-2-ol

To a solution of TiCl₄ (1 M in CH₂Cl₂, 18.2 mL) in diethyl ether at −78° C., was added methyllithium (1.6 M in diethyl ether, 11.4 mL). The resulting solution was stirred at −78° C. for 0.5 h and then added to a −78° C. solution of 1-(2-chloronaphtho[2,1-b]thien-8-yl)acetone (2.5 g, 9.1 mmol) from Step 3 above, in diethyl ether. After 5 minutes, the mixture was warmed up to 0° C. and stirred for 2 h. The reaction mixture was quenched with 1N HCl, water and ethyl acetate. The aqueous layer was extracted with ethyl acetate. The organic layer was washed with brine, dried over Na2SO4, filtered and concentrated. The crude material was used directly in the next step (Step 5 below).

### Step 5: 2-chloro-8-(2-hydroxy-2-methylpropyl)naphtho[2,1-b]thiophene-4,5-dione

To a solution of crude 1-(2-chloronaphtho[2,1-b]thien-8-yl)-2-methylpropan-2-ol from Step 4 above in acetic acid, was added CrO₃ (3.4 g). The mixture was stirred at 70° C. for 30 minutes, then quenched with water and ethyl acetate. The aqueous layer was extracted with ethyl acetate. The organic layer was washed with brine, dried over Na2SO4, filtered and concentrated. The crude material was purified by flash chromatography on silica (50% ethyl acetate in hexanes) to yield 2-chloro-8-(2-hydroxy-2-methylpropyl)naphtho[2,1-b]thiophene-4,5-dione (630 mg, 22%, 2 steps).

### Step 6: 1-[5-chloro-2-(2,6-dibromophenyl)-3H-thieno[2′,3′:3,4]naphtho[1,2-d]imidazol-8-yl]-2-methylpropan-2-ol

This imidazole was prepared as described in Step 5 of Example 24, substituting 2-chloro-8-(2-hydroxy-2-methylpropyl)naphtho[2,1-b]thiophene-4,5-dione from Step 5 above for 1-bromo-2,8-dichloronaphtho[2,1-b]thiophene-4,5-dione and heating the reaction mixture at 70° C. overnight instead of at 120° C. overnight.

### Step 7: 2-[5-chloro-8-(2-hydroxy-2-methylpropyl)-3H-thieno[2′,3′:3,4]naphtho[1,2-d]imidazol-2-yl]isophthalonitrile

This imidazole was prepared as described in Example 25, substituting 1-[5-chloro-2-(2,6-dibromophenyl)-3H-thieno[2′,3′:3,4]naphtho[1,2-d]imidazol-8-yl]-2-methylpropan-2-ol from Step 6 above for 6-bromo-5,8-dichloro-2-(2,6-dibromophenyl)-3H-thieno[2′,3′:3,4]naphtho[1,2-d]imidazole, to afford 2-[5-chloro-8-(2-hydroxy-2-methylpropyl)-3H-thieno[2′,3′:3,4]naphtho[1,2-d]imidazol-2-yl]isophthalonitrile. ¹H NMR δ (ppm) (400 MHz, Acetone-d₆): 8.41 (1H, s), 8.36 (2H, d), 8.35 (1H, d), 8.21 (1H, s), 8.01 (1H, t), 7.67 (1H, d), 3.49 (1H, s), 2.99 (2H, s), 1.27 (6H, s).

### Route B:

### Step 1: 1-(2-chloronaphtho[2,1-b]thien-8-yl)-2-methylpropan-2-ol

To a solution of 8-bromo-2-chloronaphtho[2,1-b]thiophene (1.5 g, 5 mmol) from Step 2 of Route A of Example 30 in THF (60 mL) at −78° C. was successively added methyllithium (1.6 M in diethyl ether, 472 μL) and butyllithium (2.5 M in hexanes, 2.3 mL). The mixture was stirred at −78° C. for 30 minutes, after which isobutylene oxide (670 μL) was added, followed by BF₃.OEt₂ (958 μL). The reaction mixture was stirred at −78° C. for 1 h, then quenched with 1 M HCl. The aqueous layer was extracted with ethyl acetate. The combined organic layers were washed with brine, dried over Na₂SO₄, filtered and concentrated. The material was purified by flash chromatography on silica (25% ethyl acetate in hexanes) to afford 1-(2-chloronaphtho[2,1-b]thien-8-yl)-2-methylpropan-2-ol (780 mg) as a yellow oil.

### Step 2: tert-butyl[2-(2-chloronaphtho[2,1-b]thien-8-yl)-1,1-dimethylethoxy]dimethylsilane

To a solution of 1-(2-chloronaphtho[2,1-b]thien-8-yl)-2-methylpropan-2-ol (1 g) from Step 1 above in THF, was added sodium hydride (60% dispersion in oil, 826 mg). The mixture was heated at reflux for 2 minutes, then cooled to room temperature. Tert-butyldimethylsilylchloride (3.1 g) was added and the reaction mixture heated at reflux for 2 h, then quenched with water. The aqueous layer was extracted with ethyl acetate. The combined organic layers were washed with brine, dried over Na2SO4, filtered and concentrated. The crude material was used directly for the next step (Step 3 below).

### Step 3: 8-(2-([tert-butyl(dimethyl)silyl]oxy)-2-methylpropyl)-2-chloronaphtho[2,1-b]thiophene-4,5-dione

This compound was prepared as described in Step 4 of Example 24, substituting crude tert-butyl[2-(2-chloronaphtho[2,1-b]thien-8-yl)-1,1-dimethylethoxy]dimethylsilane from Step 2 above for 1-bromo-2,8-dichloronaphtho[2,1-b]thiophene and performing the reaction at 70° C. for 20 minutes instead of at 120° C. for 2 h. The crude product was used directly in the next step (Step 4 below).

### Step 4: 8-(2-([tert-butyl(dimethyl)silyl]oxy)-2-methylpropyl)-5-chloro-2-(2,6-dibromophenyl)-3H-thieno[2′,3′:3,4]naphtho[1,2-d]imidazole

This imidazole was prepared as described in Step 5 of Example 24, substituting crude 8-(2-([tert-butyl(dimethyl)silyl]oxy)-2-methylpropyl)-2-chloronaphtho[2,1-b]thiophene-4,5-dione from Step 3 above for 1-bromo-2,8-dichloronaphtho[2,1-b]thiophene-4,5-dione and performing the reaction at 70° C. overnight instead of at 120° C. overnight. Purification by flash chromatography on silica (25% ethyl acetate in hexanes) afforded 8-(2-([tert-butyl(dimethyl)silyl]oxy)-2-methylpropyl)-5-chloro-2-(2,6-dibromophenyl)-3H-thieno[2′,3′:3,4]naphtho[1,2-d]imidazole (1.1 g, 47%).

### Step 5: 1-[5-chloro-2-(2,6-dibromophenyl)-3H-thieno[2′,3′:3,4]naphtho[1,2-d]imidazol-8-yl]-2-methylpropan-2-ol

TBAF (1 M in THF, 30 mL) was added to a flask containing 8-(2-([tert-butyl(dimethyl)silyl]oxy)-2-methylpropyl)-5-chloro-2-(2,6-dibromophenyl)-3H-thieno[2′,3′:3,4]naphtho[1,2-d]imidazole (1.1 g) from Step 4 above. The resulting solution was heated at reflux for 24 h, after which water was added to the reaction mixture. The aqueous layer was extracted with ethyl acetate, the organic layer dried over Na2SO4, filtered and concentrated. The crude product was used directly in the next reaction (Step 6 below).

### Step 6: 2-[5-chloro-8-(2-hydroxy-2-methylpropyl)-3H-thieno[2′,3′:3,4]naphtho[1,2-d]imidazol-2-yl]isophthalonitrile

This imidazole was prepared as described in Example 25, substituting crude 1-[5-chloro-2-(2,6-dibromophenyl)-3H-thieno[2′,3′:3,4]naphtho[1,2-d]imidazol-8-yl]-2-methylpropan-2-ol from Step 5 above for 6-bromo-5,8-dichloro-2-(2,6-dibromophenyl)-3H-thieno[2′,3′:3,4]naphtho[1,2-d]imidazole, to afford 2-[5-chloro-8-(2-hydroxy-2-methylpropyl)-3H-thieno[2′,3′:3,4]naphtho[1,2-d]imidazol-2-yl]isophthalonitrile.

### Example 36

### 2-(2,6-dibromophenyl)-1H-benzo[h]imidazo[4,5-f]quinoline

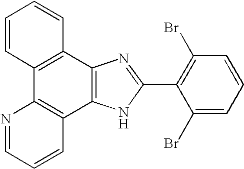

### Step 1: Benzo[h]quinoline-5,6-dione

To a solution of benzo[h]quinoline (2.64 g, 14.7 mmol) in acetic acid (60 mL), was added diiodine pentoxide (5.5 g, 16.5 mmol). The resulting solution was heated at reflux for 2 h, then poured into 10% sodium thiosulphate and ethyl acetate, and stirred for half an hour. The aqueous layer was extracted with ethyl acetate. The combined organic layers were washed with 10% sodium thiosulphate, brine, dried over anhydrous sodium sulphate and concentrated to afford crude benzo[h]quinoline-5,6-dione (3.5 g) as a brown-orange solid.

### Step 2: 2-(2,6-dibromophenyl)-1H-benzo[h]imidazo[4,5-f]quinoline

To a solution of benzo[h]quinoline-5,6-dione (2.25 g, 10.75 mmol) from Step 1 above in acetic acid (160 mL) was added ammonium acetate (17 g, 220 mmol) and 2,6-dibromobenzaldehyde (4 g, 15.2 mmol). The mixture was heated at reflux for 2 hours, then diluted in water, stirred for 1 hour and filtered. The solid was dissolved in ethyl acetate, the organic layer washed successively with water and brine, then dried over Na2SO4, filtered and concentrated. The crude material was purified by flash chromatography on silica (1-10% acetone in CH₂Cl₂) to provide 2-(2,6-dibromophenyl)-1H-benzo[h]imidazo[4,5-f]quinoline (2.67 g, 55%) as a beige solid. ¹H NMR δ (ppm) (400 MHz, DMSO-d₆ with added TFA): 9.28 (1H, d, J=8.2 Hz), 9.13 (1H, d, J=4.8 Hz), 9.03 (1H, d, J=8.1 Hz), 8.49 (1H, d, J=8.0 Hz), 7.98-7.94 (4H, m), 7.86 (1H, t, J=7.7 Hz), 7.59 (1H, t, J=8.1 Hz).

### Example 37

### 2-(1H-benzo[h]imidazo[4,5-f]quinolin-2-yl)isophthalonitrile

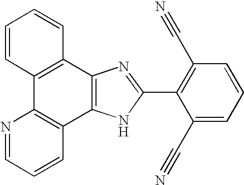

To a solution of 2-(2,6-dibromophenyl)-1H-benzo[h]imidazo[4,5-f]quinoline (206 mg, 0.45 mmol) from Step 2, Example 36 in DMF (8 mL), was added CuCN (130 mg, 1.45 mmol). The resulting mixture was heated at 80° C. for 20 h, after which further CuCN (130 mg) was added and the mixture heated at 100° C. for 7 h, then cooled to room temperature and poured into a mixture of NH4OH, brine and ethyl acetate and stirred for 16 h. The aqueous phase was extracted with ethyl acetate, the organic layer washed once with water, once with brine, dried over Na2SO4, filtered and concentrated. The material was purified by flash chromatography on silica (3-30% acetone in CH₂Cl₂) to provide 2-(1H-benzo[h]imidazo[4,5-f]quinolin-2-yl)isophthalonitrile (16 mg) as a yellow solid. ¹H NMR δ (ppm) (400 MHz, DMSO-d₆ with added TFA): 9.27 (1H, d, J=8.3 Hz), 9.08-9.04 (2H, m), 8.52 (1H, d, J=7.7 Hz), 8.46 (2H, d,

J=7.9 Hz), 8.02-7.92 (3H, m), 7.82 (1H, t, J=7.7 Hz).

### Example 38

### 2-(2,6-dibromophenyl)-1H-benzo[h]imidazo[4,5-f]quinoline 7-oxide

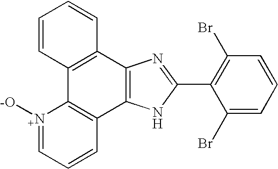

MCPBA (approx 60%, 3.5 g, 12.1 mmol) was added to a suspension of 2-(2,6-dibromophenyl)-1H-benzo[h]imidazo[4,5-f]quinoline (2.67 g, 5.89 mmol) from Example 36 in CH₂Cl₂ (120 mL). The resulting suspension was stirred at room temperature for 21 h, after which it was quenched with saturated sodium bicarbonate. A minimal amount of ethyl acetate, MeOH and THF were added and the mixture stirred at room temperature for a few minutes, then filtered to yield 2-(2,6-dibromophenyl)-1H-benzo[h]imidazo[4,5-f]quinoline 7-oxide (2.16 g, 78%) as a white solid. ¹H NMR δ (ppm) (400 MHz, DMSO-d₆ with added TFA): 10.85 (1H, d, J=8.5 Hz), 8.76 (1H, d, J=6.2 Hz), 8.51 (2H, dd, J=8.3, 14.0 Hz), 7.95-7.87 (4H, m), 7.79-7.73 (2H, m), 7.51 (1H, t, J=8.2 Hz).

### Example 39

### 2-(6-chloro-1H-benzo[h]imidazo[4,5-f]quinolin-2-yl)isophthalonitrile

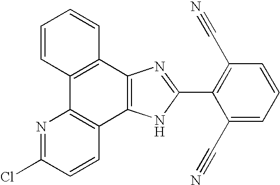

### Step 1: 6-chloro-2-(2,6-dibromophenyl)-1H-benzo[h]imidazo[4,5-f]quinoline

A suspension of 2-(2,6-dibromophenyl)-1H-benzo[h]imidazo[4,5-f]quinoline 7-oxide (340 mg) from Example 38 in phosphorus oxychloride (5 mL) was heated at 100° C. for 3 h. The reaction mixture was concentrated, the residue partitioned between ethyl acetate and 25% ammonium acetate. The aqueous phase was extracted with ethyl acetate, the organic layer washed once with brine, dried over Na2SO4, filtered and concentrated. The material was purified by flash chromatography on silica (2-30% ethyl acetate in toluene) to provide 6-chloro-2-(2,6-dibromophenyl)-1H-benzo[h]imidazo[4,5-f]quinoline (55 mg) as a white solid.

### Step 2: 2-(6-chloro-1H-benzo[h]imidazo[4,5-f]quinolin-2-yl)isophthalonitrile

To a solution of 6-chloro-2-(2,6-dibromophenyl)-1H-benzo[h]imidazo[4,5-f]quinoline (47 mg, 0.096 mmol) from Step 1 above in DMF (3 mL), was added CuCN (20 mg, 0.22 mmol). The resulting mixture was heated at 80° C. for 4 h, after which further CuCN (10 mg) was added and the mixture heated at 80° C. for 23 h, then cooled to room temperature and poured into a mixture of NH4OH, brine and ethyl acetate and stirred for 1.5 h. The aqueous phase was extracted with ethyl acetate, the organic layer washed once with water, once with brine, dried over Na2SO4, filtered and concentrated. The material was purified by flash chromatography on silica (20-70% ethyl acetate in toluene) to provide 2-(6-chloro-1H-benzo[h]imidazo[4,5-f]quinolin-2-yl)isophthalonitrile (15 mg, 41%) as a yellow solid. ¹H NMR δ (ppm) (400 MHz, DMSO-d₆ with added TFA): 9.13 (1H, d, J=8.1 Hz), 8.89 (1H, d, J=8.4 Hz), 8.49 (3H, m), 8.01 (1H, t, J=7.9 Hz), 7.94-7.88 (2H, m), 7.80 (1H, t, J=7.7 Hz).

### Example 41

### 2-(6-phenyl-1H-benzo[h]imidazo[4,5-f]quinolin-2-yl)isophthalonitrile

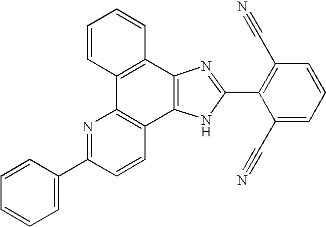

### Step 1: 2-Phenylbenzo[h]quinoline

Phenyl lithium (1.8 M in cyclohexane/ether (70/30), 2 mL, 3.6 mmol) was added to a solution of benzo[h]quinoline (430 mg, 2.41 mmol) in toluene (8 mL) at room temperature. The mixture was stirred at room temperature for 17 h after which it was cooled to 0° C., quenched with water and extracted with ethyl acetate. The organic layer was washed with brine, dried over Na2SO4, filtered and concentrated. To a solution of the crude material in methylene chloride (40 mL) was added MnO₂ (24 g, 276 mmol). The mixture was stirred at room temperature for half an hour, after which MgSO₄ (24 g) was added and the mixture stirred at room temperature for half an hour, then filtered through Celite and the filtrate concentrated. The material was purified by flash chromatography on silica (2-5% ether in hexanes) to provide 2-phenylbenzo[h]quinoline (396 mg, 64%) as a yellow oil.

### Step 2: 2-phenylbenzo[h]quinoline-5,6-dione

This dione was prepared as described in Step 1, Example 36 substituting 2-phenylbenzo[h]quinoline from Step 1 above for benzo[h]quinoline to afford 2-phenylbenzo[h]quinoline-5,6-dione.

### Step 3: 2-(2,6-dibromophenyl)-6-phenyl-1H-benzo[h]imidazo[4,5-f]quinoline

This imidazole was prepared as described in Step 2, Example 36, substituting 2-phenylbenzo[h]quinoline-5,6-dione from Step 2 above for benzo[h]quinoline-5,6-dione to afford 2-(2,6-dibromophenyl)-6-phenyl-1H-benzo[h]imidazo[4,5-f]quinoline.

### Step 4: 2-(6-phenyl-1H-benzo[h]imidazo[4,5-f]quinolin-2-yl)isophthalonitrile

This imidazole was prepared as described in Example 37, substituting 2-(2,6-dibromophenyl)-6-phenyl-1H-benzo[h]imidazo[4,5-f]quinoline from Step 3 above for 2-(2,6-dibromophenyl)-1H-benzo[h]imidazo[4,5-f]quinoline and using 4 equivalents of CuCN, to afford 2-(6-phenyl-1H-benzo[h]imidazo[4,5-f]quinolin-2-yl)isophthalonitrile. ¹H NMR δ (ppm) (400 MHz, DMSO-d₆ with added TFA): 9.46 (1H, d, J=8.0 Hz), 8.90 (1H, d, J=8.5 Hz), 8.51-8.41 (6H, m), 7.99 (1H, t, J=7.9 Hz), 7.89 (1H, t, J=6.9 Hz), 7.80 (1H, t, J=7.1 Hz), 7.60 (2H, t, J=7.5 Hz), 7.51 (1H, t, J=7.3 Hz).

### Example 42

### 6-chloro-2-(2,6-dibromophenyl)-9-methoxy-1H-benzol[h]imidazo[4,5-f]quinoline

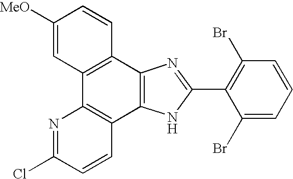

### Step 1: N,N-diethyl-4-methoxy-2-(3-methylpyridin-2-yl)benzamide

A three-necked flask charged with Pd(PPh₃)₄ (10.5 g, 9 mmol) was purged with nitrogen for 15 minutes. DME (500 mL) was then added, followed by 2-bromo-3-methylpyridine (20 mL, 0.18 mol). The solution was stirred at room temperature for 10 minutes, after which a solution of {2-[(diethylamino)carbonyl]-5-methoxyphenyl}boronic acid (0.28 mol, Can. J. Chem. 2000, 78, 905) in DME (300 mL) was added via addition funnel, followed by addition of sodium carbonate (2 M, 550 mL), also via addition funnel. The mixture was stirred at reflux for 5 h, after which it was quenched with 25% ammonium acetate. The aqueous phase was extracted with ethyl acetate, the organic layer washed with brine, dried over Na2SO4, filtered and concentrated. The material was purified by flash chromatography on silica (40-90% ethyl acetate in hexanes) to provide N,N-diethyl-4-methoxy-2-(3-methylpyridin-2-yl)benzamide (53.8 g, quant.) as a yellow solid.

### Step 2: N,N-diethyl-4-methoxy-2-(3-methyl-1-oxidopyridin-2-yl)benzamide

A suspension of N,N-diethyl-4-methoxy-2-(3-methylpyridin-2-yl)benzamide from Step 1 above (1.95 g, 6.53 mmol) and MCPBA (approx 60%, 6 g, 20 mmol) in CH₂Cl₂ (60 mL) was stirred at room temperature for 22 h, after which it was quenched with 25% ammonium acetate. The aqueous phase was extracted several times with ethyl acetate, the organic layer washed with brine, dried over Na2SO4, filtered and concentrated. The material was purified by flash chromatography on silica (10-50% ethanol in ethyl acetate) to provide N,N-diethyl-4-methoxy-2-(3-methyl-1-oxidopyridin-2-yl)benzamide (1.37 g, 67%) as a brown oil.

### Step 3: 2-(6-chloro-3-methylpyridin-2-yl)-N,N-diethyl-4-methoxybenzamide

Phosphorus oxychloride (11 mL, 118 mmol) was added to DMF (130 mL) at 0° C. The solution was stirred at room temperature for 10 minutes, after which a solution of N,N-diethyl-4-methoxy-2-(3-methyl-1-oxidopyridin-2-yl)benzamide from Step 2 above (17.11 g, 54.4 mmol) in DMF (250 mL) was added via cannula. The mixture was warmed to room temperature, stirred for 10 minutes, then placed in an oil bath at 100° C. for 5 minutes. The reaction mixture was cooled to room temperature and poured into 25% ammonium acetate. The aqueous phase was extracted with ethyl acetate, the organic layer washed once with water, once with brine, dried over Na2SO4, filtered and concentrated. The material was purified by flash chromatography on silica (50-70% ethyl acetate in hexanes) to provide 2-(6-chloro-3-methylpyridin-2-yl)-N,N-diethyl-4-methoxybenzamide (10.93 g, 60%) as a light orange solid.

### Step 4: 2-chloro-9-methoxybenzo[h]quinolin-6-ol

Butyllithium (2.5 M in hexanes, 600 μL) was added to a 0° C. solution of diisopropylamine (220 μL, 1.57 mmol) in THF (5 mL). The mixture was stirred at 0° C. for 20 min, after which a solution of 2-(6-chloro-3-methylpyridin-2-yl)-N,N-diethyl-4-methoxybenzamide (200 mg, 0.6 mmol) from Step 3 above in THF (2 mL) was added via cannula. The reaction mixture was stirred at 0° C. for 10 min, then warmed to room temperature and stirred for 1 h. The reaction mixture was re-cooled to 0° C., KHMDS (0.5 M in toluene, 2.5 mL) was added, the reaction mixture warmed to room temperature, stirred for 0.5 h, then quenched with 25% ammonium acetate. The aqueous phase was extracted with ethyl acetate, the organic layer washed with brine, dried over Na2SO4, filtered and concentrated. The material was purified by flash chromatography on silica (10% ethyl acetate in hexanes) to provide 2-chloro-9-methoxybenzo[h]quinolin-6-ol (115 mg, 74%) as a beige solid.

This carbinol could also be prepared by using the following procedure:

Step 4-a: 2-(3-methoxyphenyl)nicotinaldehyde

A round-bottomed flask charged with 2-bromonicotinaldehyde (10.45 g, 56 mmol), (3-methoxyphenyl)boronic acid (17.1 g, 113 mmol) and Pd(PPh₃)₄ (3.3 g, 2.86 mmol) was purged with nitrogen for 15 minutes. DME (300 mL) and sodium carbonate (2M, 90 mL) were then added and the mixture heated at reflux for 17 h, after which it was quenched with 25% ammonium acetate. The aqueous phase was extracted with ethyl acetate, the organic layer washed with brine, dried over Na2SO4, filtered and concentrated. The material was purified by flash chromatography on silica (30-70% ethyl acetate in hexanes) to provide 2-(3-methoxyphenyl)nicotinaldehyde (15.14 g, quant.) as a yellow solid.

### Step 4-b: Methyl [2-(3-methoxyphenyl)pyridine-3-yl]acetate

Butyllithium (2.5 M in hexanes, 30.2 mL) was added to a −78° C. solution of 1,3-dithian-2-yl(trimethyl)silane (14.4 mL, 75.6 mmol) in THF (80 mL) via addition funnel. After complete addition, the reaction mixture was stirred at −78° C. for 0.5 h. To this solution was added a solution of 2-(3-methoxyphenyl)nicotinaldehyde (14.62 g, 68.5 mmol) from Step 4-a above in THF (50 mL) via cannula. The reaction mixture was warmed to room temperature and stirred for 1 h 15 min after which it was quenched with 25% ammonium acetate. The aqueous phase was extracted with ethyl acetate, the organic layer washed with brine, dried over Na2SO4, filtered and concentrated. To the crude product dissolved in MeOH (560 mL) and water (62 mL) was added mercury (II) chloride (38 g, 140 mmol). The reaction mixture was heated at 85° C. for 6 h, then cooled to room temperature, filtered through Celite and the filtrate concentrated. The residue was partitioned between ethyl acetate and 25% ammonium acetate. The aqueous phase was extracted with ethyl acetate, the organic layer washed with brine, dried over Na2SO4, filtered and concentrated. The material was purified by flash chromatography on silica (30-50% ethyl acetate in hexanes) to provide methyl [2-(3-methoxyphenyl)pyridine-3-yl]acetate (10.7 g, 61%) as a yellow oil.

### Step 4-c: Methyl [2-(3-methoxyphenyl)-1-oxidopyridin-3-yl]acetate

A suspension of methyl [2-(3-methoxyphenyl)pyridine-3-yl]acetate from Step 4-b above (10.7 g, 41.6 mmol) and MCPBA (approx 60%, 36 g, 125 mmol) in CH₂Cl₂ (350 mL) was stirred at room temperature for 20 h, after which it was quenched with 25% ammonium acetate and ethyl acetate. Ethanol was added to solubilize the particulate matter. The aqueous phase was extracted several times with ethyl acetate, the organic layer washed with brine, dried over Na2SO4, filtered and concentrated. The material was purified by flash chromatography on silica (30-50% ethanol in ethyl acetate) to provide methyl [2-(3-methoxyphenyl)-1-oxidopyridin-3-yl]acetate (11.45 g, quant.) as an orange syrup.

### Step 4-d: Methyl [6-chloro-2-(3-methoxyphenyl)pyridine-3-yl]acetate

Phosphorus oxychloride (8.2 mL, 87.9 mmol) was added to DMF (36 mL) at 0° C. The solution was stirred at room temperature for 10 minutes, after which a solution of methyl [2-(3-methoxyphenyl)-1-oxidopyridin-3-yl]acetate (11 g, 40.2 mmol) from Step 4-c above in a mixture of toluene (18 mL) and DMF (10 mL) was added via cannula. The mixture was warmed to room temperature, stirred for 10 minutes, then placed in an oil bath at 100° C. for 10 minutes. The reaction mixture was cooled to room temperature and poured into 25% ammonium acetate. The aqueous phase was extracted with ethyl acetate, the organic layer washed once with water, once with brine, dried over Na2SO4, filtered and concentrated. The material was purified by flash chromatography on silica (10-30% ethyl acetate in hexanes) to provide methyl [6-chloro-2-(3-methoxyphenyl)pyridine-3-yl]acetate (6.7 g, 57%) as a yellow oil

### Step 4-e: [6-chloro-2-(3-methoxyphenyl)pyridine-3-yl)acetic acid

A mixture of methyl [6-chloro-2-(3-methoxyphenyl)pyridine-3-yl]acetate (6.7 g, 22.9 mmol) from Step 4-d above in a mixture of THF (100 mL)/MeOH (35 mL)/water (35 mL) was cooled to 0° C. To this mixture was added sodium hydroxide (2N, 35 mL). The reaction mixture was warmed to room temperature, stirred for 20 min, then quenched with HCl (2 N, 35 mL) and concentrated. The residue was partitioned between ethyl acetate and 25% ammonium acetate. The aqueous phase was extracted several times with ethyl acetate, the organic layer washed with brine, dried over Na2SO4, filtered and concentrated to afford [6-chloro-2-(3-methoxyphenyl)pyridine-3-yl)acetic acid (6.22 g, 98%) as a yellow solid.

### Step 4-f: 1-{[6-chloro-2-(3-methoxyphenyl)pyridin-3-yl]acetyl}-1H-1,2,3-benzotriazole

Triethylamine (1.8 mL, 12.9 mmol) was added to a room temperature solution of [6-chloro-2-(3-methoxyphenyl)pyridine-3-yl)acetic acid (2.55 g, 9.18 mmol) from Step 4-e above and 1-(methylsulfonyl)-1H-1,2,3-benzotriazole (1.82 g, 9.28 mmol, JOC, 2002, 65, 8210) in THF (50 mL). The mixture was heated at reflux for 19 h after which it was concentrated. The residue was partitioned between ethyl acetate and 25% ammonium acetate. The aqueous phase was extracted with ethyl acetate, the organic layer washed with brine, dried over Na2SO4 and filtered. The material was purified by flash chromatography on silica (10-20% ethyl acetate in hexanes) to provide 1-{[6-chloro-2-(3-methoxyphenyl)pyridin-3-yl]acetyl}-1H-1,2,3-benzotriazole (1.52 g, 44%) as a yellow solid.

### Step 4-g: 2-chloro-9-methoxybenzo[h]quinolin-6-ol

To a solution of 1-{[6-chloro-2-(3-methoxyphenyl)pyridin-3-yl]acetyl}-1H-1,2,3-benzotriazole (1.51 g, 3.98 mmol) from Step 4-f above in dichloroethane (100 mL) was added aluminium trichloride (1.62 g, 12.1 mmol). The mixture was heated at 70° C. for 0.5 h, then quenched with 25% ammonium acetate. The aqueous phase was extracted with ethyl acetate, the organic layer washed with brine, dried over Na2SO4, filtered and concentrated. The material was purified by flash chromatography on silica (5-10% ethyl acetate in toluene) to provide 2-chloro-9-methoxybenzo[h]quinolin-6-ol (927 mg, 90%).

### Step 5: 2-chloro-9-methoxybenzo[h]quinoline-5,6-dione

A mixture of 2-chloro-9-methoxybenzo[h]quinolin-6-ol (300 mg, 1.15 mmol) from Step 4 above and N,N′-Bis(salicylidene)ethylenediaminocobalt (II) hydrate (40 mg, 0.12 mmol) in DMF (8 mL) was stirred at room temperature for 3 h, with air bubbled through the reaction mixture. The mixture was then quenched with 25% ammonium acetate. The aqueous phase was extracted with ethyl acetate, the organic layer washed once with water, once with brine, dried over Na2SO4, filtered and concentrated to afford 2-chloro-9-methoxybenzo[h]quinoline-5,6-dione (300 mg, 95%) as an orange solid.

### Step 6: 6-chloro-2-(2,6-dibromophenyl)-9-methoxy-1H-benzol[h]imidazo[4,5-f]quinoline

To a solution of 2-chloro-9-methoxybenzo[h]quinoline-5,6-dione (300 mg, 1.09 mmol) from Step 5 above in acetic acid (40 mL) was added ammonium acetate (1.7 g, 22 mmol) and 2,6-dibromobenzaldehyde (440 mg, 1.67 mmol). The mixture was heated at reflux for 45 min, then poured into 25% ammonium acetate, stirred for 1 hour and filtered. The solid was dissolved in ethyl acetate, the organic layer washed successively with water and brine, then dried over Na2SO4, filtered and concentrated. The crude material was purified by flash chromatography on silica (5-20% ethyl acetate in toluene) to provide 6-chloro-2-(2,6-dibromophenyl)-9-methoxy-1H-benzol[h]imidazo[4,5-f]quinoline (480 mg, 85%) as a beige solid. ¹H NMR δ (ppm) (400 MHz, DMSO-d₆ with added TFA): 8.79 (1H, d, J=8.5 Hz), 8.52 (1H, d, J=2.6 Hz), 8.37 (1H, d, J=8.8 Hz), 7.97 (2H, d, J=8.2 Hz), 7.88 (1H, d, J=8.5 Hz), 7.60-7.56 (2H, m), 4.01 (3H, s).

### Example 43

### 2-(6-chloro-9-methoxy-3H-benzo[h]imidazo[4,5-f]quinolin-2-yl)isophthalonitrile

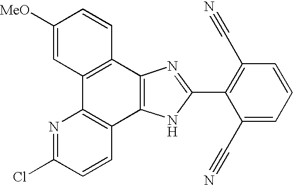

To a solution of 6-chloro-2-(2,6-dibromophenyl)-9-methoxy-1H-benzol[h]imidazo[4,5-f]quinoline (150 mg, 0.289 mmol) from Example 42 in DMF (4 mL), was added CuCN (90 mg, 1.0 mmol). The resulting mixture was heated at 80° C. for 15 h, then cooled to room temperature and poured into a mixture of NH4OH, brine, ethyl acetate and THF and stirred for 2 h. The aqueous phase was extracted with ethyl acetate, the organic layer washed once with water, once with brine, dried over Na2SO4, filtered and concentrated. The crude material was swished in toluene at 100° C. for 0.5 h and filtered to provide 2-(6-chloro-9-methoxy-3H-benzo[h]imidazo[4,5-f]quinolin-2-yl)isophthalonitrile (75 mg, 63%) as a beige solid. ¹H NMR δ (ppm) (400 MHz, DMSO-d₆ with added TFA): 8.87 (1H, d, J=8.4 Hz), 8.54 (1H, s), 8.49-8.38 (3H, m), 7.99 (1H, t, J=7.7 Hz), 7.87 (1H, d, J=8.4 Hz), 7.57 (1H, d, J=8.6 Hz), 4.03 (3H, s).

### Example 45

### 2-[9-(cyclopropylmethoxy)-6-(2-hydroxy-2-methylpropyl)-3H-benzo[h]imidazo[4,5-f]quinolin-2-yl]isophthalonitrile

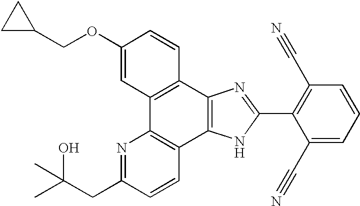

### Step 1: 6-{[tert-butyl(diphenyl)silyl]oxy}-2-chlorobenzo[h]quinolin-9-ol

KHMDS (0.5 M in toluene, 100 mL) was added to a 0° C. solution of diisopropylamine (7 mL, 50 mmol) in THF (130 mL). The mixture was stirred at 0° C. for 0.5 h and then added to a 0° C. solution of 2-(6-chloro-3-methylpyridin-2-yl)-N,N-diethyl-4-methoxybenzamide (6.6 g, 19.8 mmol) from Step 3, Example 42 in THF (60 mL) via cannula over 40 minutes. The resulting mixture was stirred at 0° C. for 0.5 h, then quenched with a few drops of saturated ammonium chloride, followed by 1 N HCl. The reaction mixture was concentrated under reduced pressure, and the residue partitioned between with 25% ammonium acetate and ethyl acetate. The aqueous phase was extracted with ethyl acetate, the organic layer washed with brine, dried over Na2SO4, filtered and concentrated to afford crude 2-chloro-9-methoxybenzo[h]quinolin-6-ol. To a solution of this crude material in DMF (180 mL), was added imidazole (4.1 g, 60.2 mmol) followed by tert-butyldiphenylchlorosilane (14.5 mL, 55.7 mmol). The resulting mixture was stirred at room temperature for 1.5 days. It was quenched with 25% ammonium acetate, the organic layer washed successively with water and brine, then dried over Na2SO4, filtered and concentrated. The crude material was passed through a quick filtration on silica (2-5% ethyl acetate in hexanes) to provide 6-{[tert-butyl(diphenyl)silyl]oxy}-2-chloro-9-methoxybenzo[h]quinoline (14.78 g).

To a solution of this product and triethylamine (1.7 mL, 12.19 mmol) in dichloroethane (300 mL) at

−10° C. was added BBr₃ via addition funnel over 40 minutes with the internal temperature maintained less than −5° C. After complete addition, the reaction mixture was warmed to 0° C., stirred for 1 h, then warmed to room temperature and stirred for 4 h. The reaction was quenched with 25% ammonium acetate, the organic layer washed with brine, dried over Na2SO4, filtered and concentrated. The crude material was purified by flash chromatography on silica (2-20% ethyl acetate in hexanes) to provide 6-{[tert-butyl(diphenyl)silyl]oxy}-2-chlorobenzo[h]quinolin-9-ol (5.8 g, 61%, 3 steps) as a beige solid.

### Step 2: 6-{[tert-butyl(diphenyl)silyloxy)-2-chloro-9-(cyclopropylmethoxy)benzo[h]quinoline

To a solution of 6-{[tert-butyl(diphenyl)silyl]oxy}-2-chlorobenzo[h]quinolin-9-ol (592 mg, 1.22 mmol) from Step 1 above in THF (15 mL) was added successively cyclopropylmethanol (180 uL, 2.25 mmol), triphenylphosphine (580 mg, 2.21 mmol) and di-tert-butylazodicarboxylate (370 mg, 1.61 mmol). The mixture was stirred at room temperature for 3 h, then quenched with 25% ammonium acetate. The organic layer was washed with brine, dried over Na2SO4, filtered and concentrated. The crude material was filtered through silica (2-30% ethyl acetate in hexanes). The material collected was swished in hexanes for 20 h and filtered to afford 6-{[tert-butyl(diphenyl)silyloxy)-2-chloro-9-(cyclopropylmethoxy)benzo[h]quinoline (660 mg, quant.) as a white solid.

### Step 3: 9-(cyclopropylmethoxy)-2-(2-hydroxy-2-methylpropyl)benzo[h]quinolin-6-ol

A round bottomed flask charged with 6-{[tert-butyl(diphenyl)silyloxy)-2-chloro-9-(cyclopropylmethoxy)benzo[h]quinoline (4.98 g, 9.25 mmol) from Step 2 above, palladium (II) acetate (210 mg, 0.93 mmol) and tri-o-tolylphosphine (570 mg, 1.87 mmol) was purged with nitrogen for 15 minutes. Toluene (20 ml) was then added, followed by isopropenylacetate (1.6 mL, 14.5 mmol) and tributyl(methoxy)stannane (4 mL, 14 mmol). The resulting mixture was heated at 100° C. for 1 h, then cooled to room temperature and further equivalents of palladium (II) acetate (210 mg), tri-o-tolylphosphine (570 mg), isopropenylacetate (1.6 mL) and tributyl(methoxy)stannane (4 mL) were added. The mixture was heated at 120° C. for 2 h, cooled to room temperature and quenched with 25% ammonium acetate. The organic layer was washed with brine, dried over Na2SO4, filtered and concentrated. The crude material was purified by flash chromatography on silica (0-5% ethyl acetate in toluene) to provide 1-[6-{tert-butyl(diphenyl)silyl]oxy-9-(cyclopropylmethoxy)benzo[h]quinolin-2-yl]acetone (2.4 g), which was used in the following procedure: To a round bottomed flask at −78° C. charged with TiCl₄ (1 M in CH₂Cl₂, 18 mL) was added methyllithium (1.6 M in diethyl ether, 11.2 mL) via addition funnel. The resulting deep red solution was stirred at −78° C. for 0.5 h and then added via cannula to a 0° C. solution of 1-[6-{tert-butyl(diphenyl)silyl]oxy-9-(cyclopropylmethoxy)benzo[h]quinolin-2-yl]acetone in diethyl ether (40 mL). The resulting mixture was stirred at 0° C. for 5 h, then quenched with 1 N HCl and ethyl acetate, followed by 25% ammonium acetate. The aqueous layer was extracted with ethyl acetate. The organic layer was washed with brine, dried over Na2SO4, filtered and concentrated. The crude material was purified by flash chromatography on silica (0-6% ethyl acetate in toluene) to provide 1-[6-{[tert-butyl(diphenyl)silyl]oxy)-9-(cyclopropylmethoxy)benzo[h]quinolin-2-yl]-2-methylpropan-2-ol (655 mg). To a solution of this material in THF (15 mL) was added tetrabutyl ammonium fluoride (1 M in THF, 2.3 mL). The mixture was stirred at room temperature for 50 min, then quenched with 25% ammonium acetate. The aqueous layer was extracted with ethyl acetate. The organic layer was washed with brine, dried over Na2SO4, filtered and concentrated. The crude material was purified by flash chromatography on silica (20-60% ethyl acetate in hexanes) to afford 9-(cyclopropylmethoxy)-2-(2-hydroxy-2-methylpropyl)benzo[h]quinolin-6-ol (200 mg).

### Step 4: 9-(cyclopropylmethoxy)-2-(2-hydroxy-2-methylpropyl)benzo[h]quinoline-5,6-dione

This quinone was prepared as described in Step 5, Example 42, substituting 9-(cyclopropylmethoxy)-2-(2-hydroxy-2-methylpropyl)benzo[h]quinolin-6-ol from Step 3 above for 2-chloro-9-methoxybenzo[h]quinolin-6-ol.

### Step 5: 1-[9-(cyclopropylmethoxy)-2-(2,6-dibromophenyl)-3H-benzo[h]imidazo[4,5-f]quinolin-6-yl]-2-methylpropan-2-ol

This imidazole was prepared as described in Step 6, Example 42, substituting 9-(cyclopropylmethoxy)-2-(2-hydroxy-2-methylpropyl)benzo[h]quinoline-5,6-dione from Step 4 above for 2-chloro-9-methoxybenzo[h]quinoline-5,6-dione to afford 1-[9-(cyclopropylmethoxy)-2-(2,6-dibromophenyl)-3H-benzo[h]imidazo[4,5-f]quinolin-6-yl]-2-methylpropan-2-ol.

### Step 6: 2-[9-(cyclopropylmethoxy)-6-(2-hydroxy-2-methylpropyl)-3H-benzo[h]imidazo[4,5-f]quinolin-2-yl]isophthalonitrile

This compound was prepared as described in Example 43, substituting 1-[9-(cyclopropylmethoxy)-2-(2,6-dibromophenyl)-3H-benzo[h]imidazo[4,5-f]quinolin-6-yl]-2-methylpropan-2-ol from Step 5 above for 6-chloro-2-(2,6-dibromophenyl)-9-methoxy-1H-benzol[h]imidazo[4,5-f]quinoline to yield 2-[9-(cyclopropylmethoxy)-6-(2-hydroxy-2-methylpropyl)-3H-benzo[h]imidazo[4,5-f]quinolin-2-yl]isophthalonitrile. ¹H NMR δ (ppm) (400 MHz, Acetone-d₆, mixture of tautomers): 13.18-13.10 (1 Ha, b, bs), 8.88 (0.6 Ha, d, J=8.16 Hz), 8.76-8.67 (1.4 Ha, b, m), 8.61 (0.3 Hb, d, J=8.75 Hz), 8.39-8.31 (2.6 Ha, b, m), 7.98 (1 Ha, b, t, J=7.93 Hz), 7.74-7.66 (1 Ha, b, m), 7.51-7.43 (1 Ha, b, m), 4.92 (0.6 Ha, bs), 4.77 (0.4 Hb, bs), 4.09 (2 Ha, b, d, J=6.91 Hz), 3.24 (2 Ha, b, s), 1.43-1.34 (1 Ha, b, m), 1.28 (6 Ha, b, s), 0.69-0.62 (2 Ha, b, m), 0.49-0.43 (2 Ha, b, m).

### Example 52

### Ethyl 9-(cyclopropylmethoxy)-2-(2,6-dicyanophenyl)-3H-benzo[h]imidazo[4,5-f]quinoline-6-carboxylate

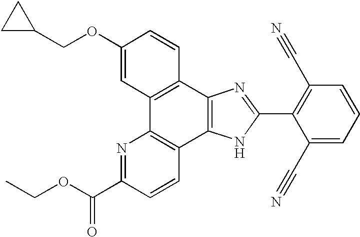

### Step 1: Ethyl 6-{[tert-butyl(diphenyl)silyl]oxy}-9-(cyclopropylmethoxybenzo[h]quinoline-2-carboxylate

A solution of 6-{[tert-butyl(diphenyl)silyloxy)-2-chloro-9-(cyclopropylmethoxy)benzo[h]quinoline (1.52 g, 2.83 mmol) from Step 2, Example 45, palladium (II) acetate (65 mg, 0.28 mmol) and dppf (320 mg, 0.58 mmol) in a mixture of ethanol (20 mL) and DMF (20 mL) was purged under vacuum and backfilled with carbon monoxide 3 times. Triethylamine (820 μL, 3.7 mmol) was added and the mixture heated at 90° C. for 3 h, under an atmosphere of carbon monoxide. The reaction mixture was quenched by pouring into 25% ammonium acetate and ethyl acetate. The aqueous layer was extracted with ethyl acetate. The organic layer was washed once with water, once with brine, dried over Na2SO4, filtered and concentrated. The crude material was purified by flash chromatography on silica (0-20% ethyl acetate in toluene) to afford ethyl 6-{[tert-butyl(diphenyl)silyl]oxy}-9-(cyclopropylmethoxybenzo[h]quinoline-2-carboxylate (380 mg) in addition to ethyl 9-(cyclopropylmethoxy)-6-hydroxybenzo[h]quinoline-2-carboxylate (300 mg).

### Step 2: Ethyl 9-(cyclopropylmethoxy)-6-hydroxybenzo[h]quinoline-2-carboxylate

To a solution of 6-{[tert-butyl(diphenyl)silyl]oxy}-9-(cyclopropylmethoxybenzo[h]quinoline-2-carboxylate (380 mg, 0.659 mmol) from Step 1 above in THF (7 mL) was added tetrabutyl ammonium fluoride (1 M in THF, 670 μL). The mixture was stirred at room temperature for 2 h, then quenched with 25% ammonium acetate. The aqueous layer was extracted with ethyl acetate. The organic layer was washed with brine, dried over Na2SO4, filtered and concentrated. The crude material was purified by flash chromatography on silica (5-20% ethyl acetate in toluene) to afford ethyl 9-(cyclopropylmethoxy)-6-hydroxybenzo[h]quinoline-2-carboxylate (124 mg).

### Step 3: Ethyl 9-(cyclopropylmethoxy)-5,6-dioxo-5,6-dihydrobenzo[h]quinoline-2-carboxylate

This quinone was prepared as described in Step 5, Example 42, substituting ethyl 9-(cyclopropylmethoxy)-6-hydroxybenzo[h]quinoline-2-carboxylate from Step 2 above for 2-chloro-9-methoxybenzo[h]quinolin-6-ol.

### Step 4: Ethyl 9-(cyclopropylmethoxy)-2-(2,6-dibromophenyl)-3H-benzo[h]imidazo[4,5-f]quinoline-6-carboxylate

This imidazole was prepared as described in Step 6, Example 42, substituting ethyl 9-(cyclopropylmethoxy)-5,6-dioxo-5,6-dihydrobenzo[h]quinoline-2-carboxylate from Step 3 above for 2-chloro-9-methoxybenzo[h]quinoline-5,6-dione to afford ethyl 9-(cyclopropylmethoxy)-2-(2,6-dibromophenyl)-3H-benzo[h]imidazo[4,5-f]quinoline-6-carboxylate.

### Step 5: Ethyl 9-(cyclopropylmethoxy)-2-(2,6-dicyanophenyl)-3H-benzo[h]imidazo[4,5-f]quinoline-6-carboxylate

This imidazole was prepared as described in Example 43 substituting ethyl 9-(cyclopropylmethoxy)-2-(2,6-dibromophenyl)-3H-benzo[h]imidazo[4,5-f]quinoline-6-carboxylate from Step 4 above for 6-chloro-2-(2,6-dibromophenyl)-9-methoxy-1H-benzol[h]imidazo[4,5-f]quinoline to yield ethyl 9-(cyclopropylmethoxy)-2-(2,6-dicyanophenyl)-3H-benzo[h]imidazo[4,5-f]quinoline-6-carboxylate. ¹H NMR δ (ppm) (400 MHz, DMSO-d₆ with added TFA): 8.95 (1H, d, J=8.35 Hz), 8.75 (1H, d, J=2.67 Hz), 8.46 (2H, d, J=7.93 Hz), 8.40 (2H, dd, J=8.57, 2.11 Hz), 7.98 (1H, t, J=7.94 Hz), 7.59 (1H, dd, J=8.82, 2.67 Hz), 4.47 (2H, q, J=7.09 Hz), 4.10 (2H, d, J=6.94 Hz), 1.44 (3H, t, J=7.10 Hz), 1.38-1.31 (1H, m), 0.67-0.60 (2H, m), 0.48-0.42 (2H, m).

### Example 53

### 2-[9-(cyclopropylmethoxy)-6-(1-ethyl-1-hydroxypropyl)-3H-benzo[h]imidazo[4,5-f]quinolin-2-yl] isophthalonitrile

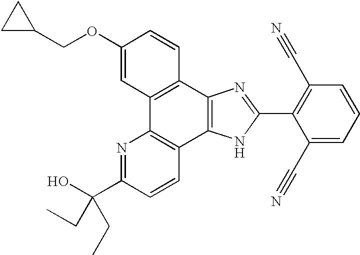

### Step 1: 3-[9-(cyclopropylmethoxy)-2-(2,6-dibromophenyl)-3H-benzo[h]imidazo[4,5-f]quinolin-6-yl]pentan-3-ol

To a −78° C. solution of ethyl 9-(cyclopropylmethoxy)-2-(2,6-dibromophenyl)-3H-benzo[h]imidazo[4,5-f]quinoline-6-carboxylate (340 mg, 0.57 mmol) from Step 4, Example 52 in CH₂Cl₂ (10 mL) was added ethylmagnesium bromide (3 M in diethyl ether, 1.4 mL). The mixture was warmed to −45° C., stirred for 0.5 h, then warmed to between −30 and −25° C. The reaction mixture was stirred at this temperature for 4 h, then quenched with 25% ammonium acetate. The aqueous layer was extracted with ethyl acetate. The organic layer was washed with brine, dried over Na2SO4, filtered and concentrated. The crude material was purified by flash chromatography on silica (5-40% ethyl acetate in toluene) to afford 3-[9-(cyclopropylmethoxy)-2-(2,6-dibromophenyl)-3H-benzo[h]imidazo[4,5-f]quinolin-6-yl]pentan-3-ol (300 mg, 86%).

### Step 2: 2-[9-(cyclopropylmethoxy)-6-(1-ethyl-1-hydroxypropyl)-3H-benzo[h]imidazo[4,5-f]quinolin-2-yl]isophthalonitrile

This imidazole was prepared as described in Example 43, substituting 3-[9-(cyclopropylmethoxy)-2-(2,6-dibromophenyl)-3H-benzo[h]imidazo[4,5-f]quinolin-6-yl]pentan-3-ol from Step 1 above for 6-chloro-2-(2,6-dibromophenyl)-9-methoxy-1H-benzol[h]imidazo[4,5-f]quinoline to afford 2-[9-(cyclopropylmethoxy)-6-(1-ethyl-1-hydroxypropyl)-3H-benzo[h]imidazo[4,5-f]quinolin-2-yl]isophthalonitrile. ¹H NMR δ (ppm) (400 MHz, DMSO-d₆ with added TFA): 8.77 (1H, d, J=8.41 Hz), 8.69 (1H, d, J=2.62 Hz), 8.44 (2H, d, J=7.90 Hz), 8.38 (1H, d, J=8.75 Hz), 8.01-7.93 (2H, m), 7.52 (1H, dd, J=8.74, 2.62 Hz), 4.08 (2H, d, J=6.92 Hz), 2.18-2.06 (2H, m), 1.96-1.85 (2H, m), 1.39-1.26 (1H, m), 0.70 (6H, t, J=7.29 Hz), 0.66-0.57 (2H, m), 0.49-0.38 (2H, m).

### Example 56

### 2-(2-chloro-6-fluorophenyl)-1H-[1]benzothieno[3′,2′:3,4]naphtho[1,2-d]imidazole

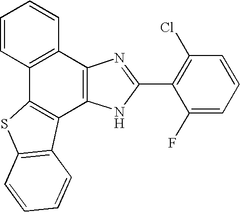

### Step 1: Methyl [2-(1-benzothien-2-yl)phenyl]acetate

A mixture of methyl (2-bromophenyl)acetate (344 mg, 1.5 mmol) from Step 1 of Example 4, 1-benzothien-2-ylboronic acid (334 mg, 1.87 mmol), Pd(PPh₃)₄ (52 mg, 0.045 mmol) and cesium fluoride (456 mg, 3 mmol) in DME (8 mL) was purged under nitrogen atmosphere for 10 min, followed by heating at reflux for 5 h. The reaction mixture was diluted with ethyl acetate. The organic layer was washed with water (3 times), dried over Na2SO4, filtered and concentrated. The material was purified by flash chromatography on silica (100% toluene) to afford methyl [2-(1-benzothien-2-yl)phenyl]acetate (328 mg) as a light yellow syrup.

### Step 2: [2-(1-benzothien-2-yl)phenyl]acetic acid

To a solution of methyl [2-(1-benzothien-2-yl)phenyl]acetate (325 mg) from Step 1 above in THF (10 mL)/methanol (10 mL), was added sodium hydroxide (1N, 5 mL). The mixture was stirred at 55° C. for 1.5 h, after which it was cooled to room temperature and concentrated to a small volume. The residue was diluted with water and acidified with HCl (1 N) to yield a precipitate which was filtered to afford [2-(1-benzothien-2-yl)phenyl]acetic acid (235 mg) as a white solid.

### Step 3: Benzo[b]naphtho[2,1-d]thiophene-6-ol

A mixture of [2-(1-benzothien-2-yl)phenyl]acetic acid (230 mg) from Step 2 above and thionyl chloride (314 μL, 4.3 mmol) in 1,2-dichloroethane (5 mL) was heated at reflux for 2.5 h, after which the reaction mixture was evaporated to dryness. The residue was co-evaporated with 1,2-dichloroethane (3 mL) to provide a brick-coloured solid which was suspended in 1,2-dichloroethane (6 mL). To this suspension was added aluminium trichloride (170 mg, 1.28 mmol) at room temperature. The mixture was stirred at room temperature for 1 h, then diluted with water and CH₂Cl₂. The aqueous layer was extracted with CH₂Cl₂, the organic layer dried over Na2SO4, filtered and concentrated. The material was purified by flash chromatography on silica (100% toluene) to provide benzo[b]naphtho[2,1-d]thiophene-6-ol (56 mg) as a beige solid.

### Step 4: Benzo[b]naphtho[2,1-d]thiophene-5,6-dione

A mixture of benzo[b]naphtho[2,1-d]thiophene-6-ol (48.5 mg, 0.19 mmol) from Step 4 above and N,N′-Bis(salicylidene)ethylenediaminocobalt (II) hydrate [Co(SALEN)₂] (6.5 mg, 0.02 mmol) in DMF (4 mL) was stirred at room temperature overnight, with air bubbled through the reaction mixture. The mixture was then quenched with water and stirred for 15 min. The aqueous layer was extracted with ethyl acetate, the combined organic layers washed with water, dried over Na2SO4, filtered and concentrated to afford benzo[b]naphtho[2,1-d]thiophene-5,6-dione (44 mg) as a dark red solid.

### Step 5: 2-(2-chloro-6-fluorophenyl)-1H-[1]benzothieno[3′,2′:3,4]naphtho[1,2-d]imidazole

A mixture of benzo[b]naphtho[2,1-d]thiophene-5,6-dione (36 mg, 0.13 mmol) from Step 4 above, 2-chloro-6-fluorobenzaldehyde (28.5 mg, 0.18 mmol) and ammonium acetate (105 mg, 1.36 mmol) in acetic acid (4 mL) was heated at 110° C. for 5 h, after which it was diluted with water and stirred for 10 minutes. The resulting precipitate was filtered and the solid obtained was washed with water, then purified on 2 prep chromatography plates (eluent 30% ethyl acetate in hexanes) to afford 2-(2-chloro-6-fluorophenyl)-1H-[1]benzothieno[3′,2′:3,4]naphtho[1,2-d]imidazole (40 mg) as a light yellow solid. ¹H NMR δ (ppm) (400 MHz, Acetone-d₆): 12.85 (1H, bs), 9.25 (1H, d), 8.54 (1H, d), 8.31 (1H, d), 8.16 (1H, d), 7.79-7.58 (6H, m), 7.43 (1H, t).

### Example 57

### 2-(2-chloro-6-fluorophenyl)-1H-[1]benzothieno[2′,3′:3,4]naphtho[1,2-d]imidazole

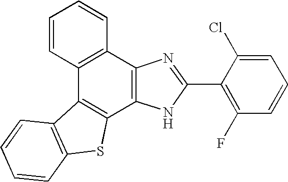

### Step 1: Methyl [2-(1-benzothien-3-yl)phenyl]acetate

This compound was prepared as described in Step 1 of Example 56, substituting 1-benzothien-3-ylboronic acid for 1-benzothien-2-ylboronic acid.

### Step 2: [2-(1-benzothien-3-yl)phenyl]acetic acid

This acid was prepared as described in Step 2 of Example 56, substituting methyl [2-(1-benzothien-3-yl)phenyl]acetate from Step 1 above for methyl [2-(1-benzothien-2-yl)phenyl]acetate.

### Step 3: Benzo[b]naphtho[1,2-d]thiophene-6-ol

To a solution of [2-(1-benzothien-3-yl)phenyl]acetic acid (1.17 mmol) from Step 2 above in THF (6 mL) at 0° C. was added oxalyl chloride (297 mg, 2.34 mmol), followed by DMF (1 drop). The resulting solution was stirred at 0° C. for 1 h and then concentrated at reduced pressure (at 40° C.). The residue was co-evaporated with 1,2-dichloroethane (5 mL) and then dried under high vacuum for 30 minutes. The crude acid chloride was dissolved in 1,2-dichloroethane (8 mL). To this solution was added aluminium trichloride (213 mg, 1.6 mmol) at room temperature. The mixture was stirred at room temperature for 1 h, then diluted with CH₂Cl₂ and water. The aqueous layer was extracted with CH₂Cl₂, the organic layer dried over Na2SO4, filtered and concentrated. The material was purified by flash chromatography on silica (30% ethyl acetate in hexanes) to afford benzo[b]naphtho[1,2-d]thiophene-6-ol (96 mg).

### Step 4: Benzo[b]naphtho[1,2-d]thiophene-5,6-dione

This dione was prepared as described in Step 4 of Example 56, substituting benzo[b]naphtho[1,2-d]thiophene-6-ol from Step 3 above for benzo[b]naphtho[2,1-d]thiophene-6-ol.

### Step 5: 2-(2-chloro-6-fluorophenyl)-1H-[1]benzothieno[2′,3′:3,4]naphtho[1,2-d]imidazole

This imidazole was prepared as described in Step 5 of Example 56, substituting benzo[b]naphtho[1,2-d]thiophene-5,6-dione from Step 4 above for benzo[b]naphtho[2,1-d]thiophene-5,6-dione to afford 2-(2-chloro-6-fluorophenyl)-1H-[1]benzothieno[2′,3′:3,4]naphtho[1,2-d]imidazole as a beige solid.

¹H NMR δ (ppm) (400 MHz, Acetone-d₆, mixture of tautomers): 12.9 (0.5 Ha, bs), 12.75 (0.5 Hb, bs), 9.31-9.29 (1 Ha, b, m), 9.09-9.02 (1 Ha, b, m), 8.88 (0.2 Hb, d), 8.58 (0.8 Hb, d), 8.21 (1 Ha, b, d), 7.85-7.67 (4 Ha, b, m), 7.59-7.56 (2 Ha, b, m), 7.42 (1 Ha, b, t).

### Example 58

### 2-(2-chloro-6-fluorophenyl)-1,12-dihydrobenzo[c]imidazo[4,5-a]carbazole

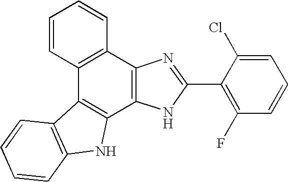

### Step 1: Methyl [2-(1H-indol-3-yl)phenyl]acetate

A mixture of methyl (2-bromophenyl)acetate (2.52 g, 11 mmol) from Step 1 of Example 4, 1H-indole (1.17 g, 10 mmol), palladium (II) acetate (22.5 mg, 0.1 mmol), P(t-Bu)₃ (61 mg, 0.3 mmol) and potassium carbonate (3 equivalents) in xylenes (40 mL) was purged under nitrogen atmosphere for 10 min, followed by heating at 110-120° C. overnight. A few drops of P(t-Bu)₃ and palladium (II) acetate (10 mg) were then added and the reaction mixture heated at 110-120° C. for 6 h. The reaction was quenched with saturated ammonium chloride and partitioned between ethyl acetate and water. The organic phase was dried over Na2SO4, filtered and concentrated. The material was purified by flash chromatography on silica (25% ethyl acetate in hexanes) to afford methyl [2-(1H-indol-3-yl)phenyl]acetate (435 mg) as a beige solid.

### Step 2: [2-(1H-indol-3-yl)phenyl]acetic acid

This acid was prepared as described in Step 2 of Example 56, substituting methyl [2-(1H-indol-3-yl)phenyl]acetate from Step 1 above for methyl [2-(1-benzothien-2-yl)phenyl]acetate.

### Step 3: 7H-benzo[c]carbazol-6-ol

To a solution of [2-(1H-indol-3-yl)phenyl]acetic acid (387 mg, 1.54 mmol) from Step 2 above in THF (15 mL) at 0° C. was added oxalyl chloride (392 mg, 3 mmol), followed by DMF (2.5 μL). The resulting suspension was stirred at 0° C. for 3 h, then warmed to room temperature and stirred for 1 h. Aluminium trichloride (133 mg) was then added and the reaction mixture stirred at room temperature for 20 minutes. The reaction was quenched with water and partitioned between ethyl acetate and water. The organic layer was dried over Na2SO4, filtered and concentrated. The material was purified by flash chromatography on silica (30% ethyl acetate in hexanes) to afford 7H-benzo[c]carbazol-6-ol (230 mg).

### Step 4: 5H-benzo[c]carbazole-5,6(7H)-dione

This dione was prepared as described in Step 4 of Example 56, substituting 7H-benzo[c]carbazol-6-ol from Step 3 above for benzo[b]naphtho[2,1-d]thiophene-6-ol.

### Step 5: 2-(2-chloro-6-fluorophenyl)-1,12-dihydrobenzo[c]imidazo[4,5-a]carbazole

This imidazole was prepared as described in Step 5 of Example 56, substituting 5H-benzo[c]carbazole-5,6(7H)-dione from Step 4 above for benzo[b]naphtho[2,1-d]thiophene-5,6-dione to afford 2-(2-chloro-6-fluorophenyl)-1,12-dihydrobenzo[c]imidazo[4,5-a]carbazole. ¹H NMR δ (ppm) (400 MHz, Acetone-d₆): 12.75 (1H, bs), 11.35 (1H, bs), 9.00 (1H, d), 8.65 (1H, d), 8.6-8.4 (1H, m), 7.9-7.63 (3H, m), 7.6-7.5 (2H, m), 7.5-7.3 (3H, m).

### Example 59

### 2-(1H-bisthieno[2,3-e:3′,2′-g]benzimidazol-2-yl)isophthalonitrile

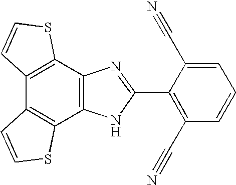

### Step 1: 3,3′-bithiophene

To a mixture of 3-bromothiophene (2 g, 12.2 mmol) and 3-thienylboronic acid (2.4 g, 18.4 mmol) in DME (100 mL), was added sodium carbonate (2M, 18.4 mL). The mixture was degassed under vacuum and backfilled with nitrogen. Pd(PPh₃)₄ (707 mg, 0.61 mmol) was added and the reaction mixture heated at reflux for 10 h, after which it was diluted in ethyl acetate and water. The aqueous layer was extracted with diethyl ether, the organic layers washed with brine, dried over Na2SO4, filtered and concentrated. The material was purified by flash chromatography on silica (10% ethyl acetate in hexanes) to afford 3,3′-bithiophene (1.1 g, 55%).

### Step 2: 2-(2,6-dibromophenyl)-1H-bisthieno[2,3-e:3′,2′-g]benzimidazole

To a solution of 3,3′-bithiophene (1.1 g, 6.6 mmol) from Step 1 above in 1,2-dichloroethane (20 mL), was added oxalyl chloride (287 μL). The reaction mixture was heated at reflux for 2 days, after which a further amount of oxalyl chloride (100 μL) was added. The reaction mixture was heated at reflux for an additional 2 days, after which a further amount of oxalyl chloride (250 μL) was added. The reaction mixture was heated at reflux for another 3 days, after which it was placed at 5° C. for 2 hours. The resulting precipitate was filtered. The solid obtained was washed with hexanes, then swished in hot ethanol for 30 minutes, filtered and dried under high vacuum to afford the corresponding dione (500 mg). A mixture of this dione (500 mg, 2.27 mmol), ammonium acetate (1.7 g, 22.7 mmol) and 2,6-dibromobenzaldehyde (778 mg, 2.95 mmol) in acetic acid (11 mL) was heated at reflux for 2 h. The reaction mixture was then cooled to room temperature, poured into water and the resulting precipitate filtered. The solid obtained was dissolved in ethyl acetate and this solution was washed successively with saturated sodium bicarbonate and brine, dried over Na2SO4, filtered and concentrated. The material was purified by flash chromatography on silica (10-100% ethyl acetate in hexanes) to afford 2-(2,6-dibromophenyl)-1H-bisthieno[2,3-e:3′,2′-g]benzimidazole (630 mg, 63%).

### Step 3: 2-(1H-bisthieno[2,3-e:3′,2′-g]benzimidazol-2-yl)isophthalonitrile

To a solution of 2-(2,6-dibromophenyl)-1H-bisthieno[2,3-e:3′,2′-g]benzimidazole (630 mg, 1.36 mmol) from Step 2 above in DMF (7 mL), was added CuCN (487 mg, 5.4 mL). The reaction mixture was stirred at 80° C. overnight, after which it was cooled to room temperature and poured into a mixture of 10% ammonium hydroxide, ethyl acetate and THF. The mixture was stirred at room temperature for 1 h. The aqueous layer was extracted with ethyl acetate, the organic layer was washed successively with 10% ammonium hydroxide and brine, dried over Na2SO4, filtered and concentrated. The material was purified by flash chromatography on silica (50-100% ethyl acetate in hexanes) to afford 2-(1H-bisthieno[2,3-e:3′,2′-g]benzimidazol-2-yl)isophthalonitrile (200 mg). ¹H NMR δ (ppm) (400 MHz, DMSO-d₆): 14.3 (1H, s), 8.45 (2H, d), 8.12 (2H, dd), 7.98 (1H, t), 7.9 (1H, d), 7.82 (1H, d).

### Example 60

### 2-(5,8-dibromo-1H-bisthieno[2,3-e:3′,2′-g]benzimidazol-2-yl)isophthalonitrile

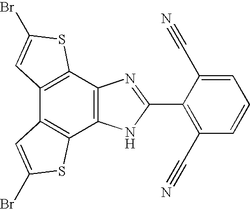

A mixture of 2-(1H-bisthieno[2,3-e:3′,2′-g]benzimidazol-2-yl)isophthalonitrile (100 mg, 0.28 mmol) from Example 59 and NBS (100 mg, 0.56 mmol) in THF (5 mL) and water (0.5 mL) was stirred at room temperature for 2 h. The reaction was quenched with 10% Na₂S₂O₃, diluted with ethyl acetate and water. The organic layer was washed with brine, dried over Na₂SO₄, filtered and concentrated. The material was purified by flash chromatography on silica to afford 2-(5,8-dibromo-1H-bisthieno[2,3-e:3′,2′-g]benzimidazol-2-yl)isophthalonitrile (25 mg). ¹H NMR δ (ppm) (400 MHz, DMSO-d₆): 14.4 (1H, s), 8.48 (2H, d), 8.3 (2H, d), 8.0 (1H, t).

### Assays for Determining Biological Activity

### Inhibition of Prostaglandin E Synthase Activity

Compounds are tested as inhibitors of prostaglandin E synthase activity in microsomal prostaglandin e synthases, whole cell and in vivo assays. These assays measure prostaglandin E2 (PGE2) synthesis using either Enzymatic Immunoassay (EIA) or mass spectrometry. Cells used for microsomal preparation are CHO-K1 cells transiently transfected with plasmids encoding the human mPGES-1 cDNA. Cells used for cell-based experiments are human A549 (which express human mPGES-1). Guinea pigs are used to test the activity of selected compounds in vivo. In all these assays, 100% activity is defined as the PGE₂ production in vehicle-treated samples. IC₅₀ and ED₅₀ represent the concentration or dose of inhibitor required to inhibit PGE₂ synthesis by 50% as compared to the uninhibited control.

### Microsomal Prostaglandin E Synthase Assay

Prostaglandin E synthase microsomal fractions are prepared from CHO-K1 cells transiently transfected with plasmid encoding the human mPGES-1 cDNA. Microsomes are then prepared and the PGES assay begins with the incubation of 5 μg/ml microsomal PGES-1 with compound or DMSO (final 1%) for 20-30 minutes at room temperature. The enzyme reactions are performed in 200 mM KPi pH 7.0, 2 mM EDTA and 2.5 mM GSH-reduced form. The enzymatic reaction is then initiated by the addition of 1 μM final PGH2 substrate prepared in isopropanol (3.5% final in assay well) and incubated at room temperature for 30 seconds. The reaction is terminated by the addition of SnCl₂ in 1N HCl (1 mg/ml final). Measurement of PGE₂ production in the enzyme reaction aliquots is done by EIA using a standard commercially available kit (Cat #: 901-001 from Assay Designs).

Data from this assay for representative compounds is shown in the table below. The potency is expressed as IC₅₀ and the value indicated is an average of at least n=3.

| Ex. | h-CHO (nM) |
|---|---|
| 1 | 20.7 |
| 2 | 10.5 |
| 12 | 3.2 |
| 15 | 1.8 |
| 28 | 1.5 |
| 33 | 237 |
| 48 | 2.1 |

### Human A549 Whole Cell Prostaglandin E Synthase Assay

### Rationale

Whole cells provide an intact cellular environment for the study of cellular permeability and biochemical specificity of anti-inflammatory compounds such as prostaglandin E synthase inhibitors. To study the inhibitory activities of these compounds, human A549 cells are stimulated with 10 ng/ml recombinant human IL-1β for 24 hours. The production of PGE₂ and PGF_{2α} are measured by EIA at the end of the incubation as readouts for selectivity and effectiveness against mPGES-1-dependent PGE₂ production.

### Methods

Human A549 cells specifically express human microsomal prostaglandin E synthase-1 and induce its expression following treatment with IL-1β for 24 hours. 2.5×10⁴ cells seeded in 100 ul/well (96-well plate) and incubated overnight under standard conditions. 100 ul of cell culture media containing 10 ng/ml IL-1β is then added to the cells followed by the addition of either 2% FBS containing RPMI or 50% FBS containing RPMI. 2 μl of drugs or vehicle (DMSO) are then added and samples are mixed immediately. Cells are incubated for 24 hours and following the incubation 175 μl of medium is harvested and assayed for PGE₂ and PGF_{2α} contents by EIA.

### Human Whole Blood Prostaglandin E Synthase Assay

### Rationale

Whole blood provides a protein and cell-rich milieu for the study of biochemical efficacy of anti-inflammatory compounds such as prostaglandin E synthase inhibitors. To study the inhibitory activities of these compounds, human blood is stimulated with lipopolysaccharide (LPS) for 24 hours to induce mPGES-1 expression. The production of prostaglandin E2 (PGE₂) and thromboxane B2 (TxB₂) are measured by EIA at the end of the incubation as readouts for selectivity and effectiveness against mPGES-1-dependent PGE₂ production.

### Methods

Human whole blood assays for mPGES-1 activity reported (Brideau, et al., Inflamm. Res., vol. 45, p. 68, 1996) are performed as described below.

Freshly isolated venous blood from human volunteers is collected in heparinized tubes. These subjects have no apparent inflammatory conditions and have not taken any NSAIDs for at least 7 days prior to blood collection. 250 μl of blood is pre-incubated with 1 ul vehicle (DMSO) or 1 ul of test compound. Bacterial LPS at 100 μg/ml (E. Coli serotype 0111:B4 diluted in 0.1% w/v bovine serum albumin in phosphate buffered saline) is then added and samples are incubated for 24 hours at 37° C. Unstimulated control blood at time zero (no LPS) is used as blank. At the end of the 24 hr incubation, the blood is centrifuged at 300 rpm for 10 min at 4° C. The plasma is assayed for PGE₂ and TxB₂ using an EIA kit as indicated above.

### In Vivo Determination of Anti-Inflammatory Activity

### Rationale

The whole animal provides an integrated physiological system to confirm the anti-inflammatory activity of test compounds characterized in vitro. To determine the activity of prostaglandin E synthase inhibitors in vivo, animals are dosed with compounds either prior or after the inflammatory stimulus, LPS. LPS is injected into the hind paw of guinea pigs and hyperalgesia measurements are recorded 4.5 and/or 6 hrs after the injection.

### Formulation of Test Compounds for Oral Dosage

Test compound is ground and made amorphous using a ball milling system. The compound is placed in an agate jar containing agate balls and spun at high speed for 10 minutes in an apparatus such as the Planetary Micro Mill Pulverisette 7 system. The jar is then opened and 0.5% methocel solution added to the ground solid. This mixture is spun again at high speed for 10 minutes. The resulting suspension is transferred to a scintillation vial, diluted with the appropriate amount of 0.5% methocel solution, sonicated for 2 minutes and stirred until the suspension was homogeneous. Alternatively, the test compound can be formulated using amorphous material obtained by any suitable chemical or mechanical technique. This amorphous solid is then mixed and stirred for a certain period of time, such as 12 hours, with a suitable vehicle, such as 0.5% methocel with 0.02 to 0.2% of sodium dodecylsulfate, prior to dosage.

### Methods

Male Hartley guinea pigs, weighing 200-250 grams are used. LPS (30 mg/kg) is injected sub-plantarly into the left hind paw of the guinea pig to produce hyperalgesia in the injected paw. Rectal temperature and paw withdrawal latency, a measure of hypersensitivity to pain (hyperalgesia), are taken prior to LPS injection and used as the baseline. Paw withdrawal latency is determined using the thermal hyperalgesia instrument (Ugo Basile Corp.). During this determination, animals are placed in an 8″×8″ plexiglas holding box atop of a glass base. A mild (223 mW/cm²) infrared light is directed toward the underside of the hind paw. The time it takes for the animal to remove its paw (indication that it feels the pain caused by the heat) is recorded. The infrared light immediately shuts off when the animal withdraws its paw from the area. The light will also shut off automatically when the time reaches 20 seconds.

### Predose Paradigm:

Test compounds are orally dosed at 5 ml/kg using an 18-gauge feeding needle. LPS (serotype 0111:B4, 10 μg) or 0.9% saline is injected into the plantar region of the left hind paw at a volume of 100 μl using a 26 gauge needle 1 hour following compound administration. Rectal temperature and thermal paw withdrawal latency are taken 4.5 hours after LPS administration. The animals are euthanized following the measurements using CO₂ and lumbar spinal cord, hind paw and blood samples collected.

### Reversal Paradigm:

Thermal paw withdrawal of each animal is determined before and 3 hours following sub-plantar injection of LPS. Animals which have received LPS and do not show a decrease in withdrawal latency at the 3 hour time point will be removed from study and euthanized. Test compounds are dosed p.o. at 5 ml/kg immediately following the thermal paw withdrawal measurement. Thermal withdrawal latency is taken 1.5 and 3 hours following compound administration (4.5 and 6 hours post-LPS administration). After the final reading, the animals are euthanized using CO2 and lumbar spinal cord and blood samples collected for prostaglandin determination by mass spectrometry and drug level, respectively.

What is claimed is:

1. A compound represented by Formula I 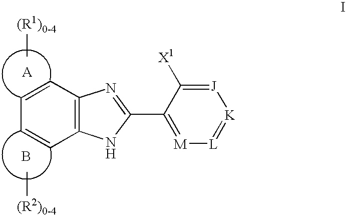
or a prodrug thereof, or a pharmaceutically acceptable salt of said compound or prodrug, wherein:
A is selected from the group consisting of: aryl, heteroaryl, heterocyclyl and cycloalkyl, or a fused analog of any of aforementioned;
B is selected from the group consisting of: aryl, heteroaryl, heterocyclyl and cycloalkyl, or a fused analog of any of the aforementioned;
with the proviso that A and B cannot both simultaneously be phenyl;
J is selected from the group consisting of —C(X²)— and —N—,
K is selected from the group consisting of —C(X³)— and —N—,
L is selected from the group consisting of —C(X⁴)— and —N—, and
M is selected from the group consisting of —C(X⁵)— and —N—,
with the proviso that at least one of J, K, L or M is other than —N—;
X¹ is selected from the group consisting of: (1) F; (2) Cl; (3) Br; (4) I; (5) —OH; (6) —N₃; (7) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (8) C₁₋₄alkoxy; (9) NR⁹R¹⁰—, NR⁹R¹⁰—C(O)—C₁₋₄alkyl-O— or NR⁹R¹⁰—C(O)—; (10) C₁₋₄alkyl-S(O)ₖ—; (11) —NO₂; (12) C₃₋₆cycloalkyl, (13) C₃₋₆cycloalkoxy; (14) phenyl, (15) carboxy; (16) C 4alkyl-O—C(O)—, and (17) —CN;
X², X³, X⁴ and X⁵ are independently selected from the group consisting of: (1) H; (2) F; (3) Cl; (4) Br; (5) I; (6) —OH; (7) —N₃; (8) C₁₋₆alkyl, C₂₋₆alkenyl or C₂₋₆alkynyl, wherein one or more of the hydrogen atoms attached to said C₁₋₆alkyl, C₂₋₆alkenyl or C₂₋₆alkynyl may be replaced with a fluoro atom, and said C₁₋₆alkyl, C₂₋₆alkenyl or C₂₋₆alkynyl may be optionally substituted with a hydroxy or oxo group; (9) C₁₋₄alkoxy; (10) NR⁹R¹⁰—, NR⁹R¹⁰—C(O)—C₁₋₄alkyl-O— or NR⁹R¹⁰—C(O)—; (11) C₁₋₄alkyl-S(O)ₖ—; (12) —NO₂; (13) C₃₋₆cycloalkyl, (14) C₃₋₆cycloalkoxy; (15) phenyl, (16) carboxy; (17) C₁₋₄alkyl-O—C(O)—, and (18) —CN;
each R¹ and R² is independently selected from the group consisting of: (1) H; (2) F; (3) Cl; (4) Br; (5) I; (6) —CN; (7) C₁₋₁₀alkyl or C₂₋₁₀alkenyl, wherein one or more of the hydrogen atoms attached to said C₁₋₁₀alkyl or C₂₋₁₀alkenyl may be replaced with a fluoro atom, or two hydrogen on adjacent carbon atoms may be joined together and replaced with —CH₂— to form a cyclopropyl group, or two hydrogen atoms on the same carbon atom may be replaced and joined together to form a spiro C₃₋₆cycloalkyl group, and wherein said C₁₋₁₀alkyl or C₂₋₁₀alkenyl may be optionally substituted with one to three substituents independently selected from the group consisting of: —OH, acetyl, acetyloxy, methoxy, ethenyl, R¹¹—O—C(O)—, R³⁵—N(R³⁶)—, R³⁷—N(R³⁸)—C(O)—, cyclopropyl, pyrrolyl, imidiazolyl, pyridyl and phenyl, said pyrrolyl, imidiazolyl, pyridyl and phenyl optionally substituted with C₁₋₄alkyl or mono-hydroxy substituted C₁₋₄alkyl; (8) C₃₋₆cycloalkyl; (9) R¹²—O—; (10) R¹³—S(O)ₖ—, (11) R¹⁴—S(O)ₖ—N(R¹⁵)—; (12) R¹⁶—C(O)—; (13) R¹⁷—N(R¹⁸)—; (14) R¹⁹—N(R²⁰)—C(O)—; (15) R²¹—N(R²²)—S(O)ₖ—; (16) R²³—C(O)—N(R²⁴)—; (17) Z-C≡C; (18) —(CH₃)C═N—OH or —(CH₃)C═N—OCH₃; (19) R³⁴—O—C(O)—; (20) R³⁹—C(O)—O—; and (21) phenyl, naphthyl, pyridyl, pyradazinyl, pyrimidinyl, pyrazinyl, pyrrolyl, pyrazolyl, imidazolyl, triazolyl, tetrazolyl, oxazolyl, isoxazolyl, oxadiazolyl, thienyl or furyl, each optionally substituted with a substituent independently selected from the group consisting of: F, Cl, Br, I, C₁₋₄alkyl, phenyl, methylsulfonyl, methylsulfonylamino, R²⁵—O—C(O)— and R²⁶—N(R²⁷)—, said C₁₋₁₄alkyl optionally substituted with 1 to 3 groups independently selected from halo and hydroxy;
each Z is independently selected from the group consisting of: (1) H; (2) C₁₋₆alkyl, wherein one or more of the hydrogen atoms attached to said C₁₋₆alkyl may be replaced with a fluoro atom, and wherein
C₁₋₆alkyl is optionally substituted with one to three substituents independently selected from: hydroxy, methoxy, cyclopropyl, phenyl, pyridyl, pyrrolyl, R²⁸—N(R²⁹)— and R³⁰—O—C(O)—; (3) —(CH₃)C═N—OH or —(CH₃)C═N—OCH₃; (4) R³¹—C(O)—; (5) phenyl; (6) pyridyl or the N-oxide thereof; (7) C₃₋₆cycloalkyl, optionally substituted with hydroxy; (8) tetrahydropyranyl, optionally substituted with hydroxy; and (9) a five-membered aromatic heterocycle containing 1 to 3 atoms independently selected from O, N or S and optionally substituted with methyl;
each R⁹, R¹⁰, R¹⁵, R²⁴ and R³² is independently selected from the group consisting of: (1) H; and
(2) C₁₋₄alkyl;
each R¹¹, R¹², R¹³, R¹⁴, R¹⁶, R²³, R²⁵, R³⁰, R³¹, R³⁴ and R³⁹ is independently selected from the group consisting of: (1) H; (2) C₁₋₄alkyl, (3) C₃₋₆cycloalkyl; (4) C₃₋₆cycloalkyl-C₁₋₄alkyl- (5) phenyl, (6) benzyl; (7) pyridyl and (8) pyridylmethyl; said C₁₋₄alkyl, C₃₋₆cycloalkyl, C₃₋₆cycloalkyl-C₁₋₄alkyl, phenyl, benzyl, pyridyl and pyridylmethyl may each be optionally substituted with 1 to 3 substituents independently selected from the group consisting of: OH, F, Cl, Br and I, and wherein said C₁₋₄alkyl may be further substituted with oxo or methoxy or both;
each R¹⁷, R¹⁸, R¹⁹, R²⁰, R²¹, R²², R²⁶, R²⁷, R²⁸, R²⁹, R³⁵, R³⁶, R³⁷ and R³⁸ is independently selected from the group consisting of: (1) H; (2) C₁₋₆alkyl; (3) C₁₋₆alkoxy; (4) OH and (5) benzyl or 1-phenylethyl; and R¹⁷ and R¹⁸, R¹⁹ and R²⁰, R²¹ and R²², R²⁶ and R²⁷, and R²⁸ and R²⁹, R³⁵ and R³⁶, and R³⁷ and R³⁸ may be joined together with the nitrogen atom to which they are attached to form a monocyclic ring of 5 or 6 carbon atoms, optionally containing one or two atoms independently selected from —O—, —S(O)ₖ— and —N(R³²)—; and
each k is independently 0, 1 or 2;
and when said compound of Formula I is a prodrug, said prodrug is represented by Formula A 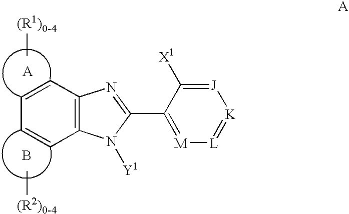
wherein:
Y¹ is selected from the group consisting of: (1) C₁₋₆alkyl; (2) PO₄—C₁₋₄alkyl-; (3) C₁₋₄alkyl-C(O)—O—CH₂—, wherein the C₁₋₄alkyl portion is optionally substituted with R³³—O—C(O)—; and (4) C₁₋₄alkyl-O—C(O)—; and
R³³ is selected from the group consisting of: (1) H; (2) C₁₋₄alkyl, (3) C₃₋₆cycloalkyl; (4) phenyl; (5) benzyl; and (6) pyridyl; said C₁₋₄alkyl, C₃₋₆cycloalkyl, phenyl, benzyl and pyridyl may each be optionally substituted with 1 to 3 substituents independently selected from the group consisting of: OH, F, Cl, Br and I.

2. The compound according to claim 1 according to Formula B 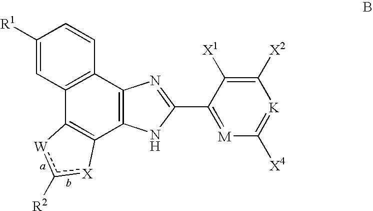
or a prodrug thereof, or a pharmaceutically acceptable salt of said compound or prodrug, wherein:
W is O or S, X is CR² and b is a double bond, or
X is O or S, W is CR² and a is a double bond.

3. The compound according to claim 2 wherein:
X² and X⁴ are H;
K is CH or N;
M is —C(X⁵)—; and
X¹ and X⁵ are independently selected from the group consisting of: (1) F; (2) Cl; (3) Br; (4) I; and (5) CN.

4. The compound according to claim 3 wherein:
R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl,

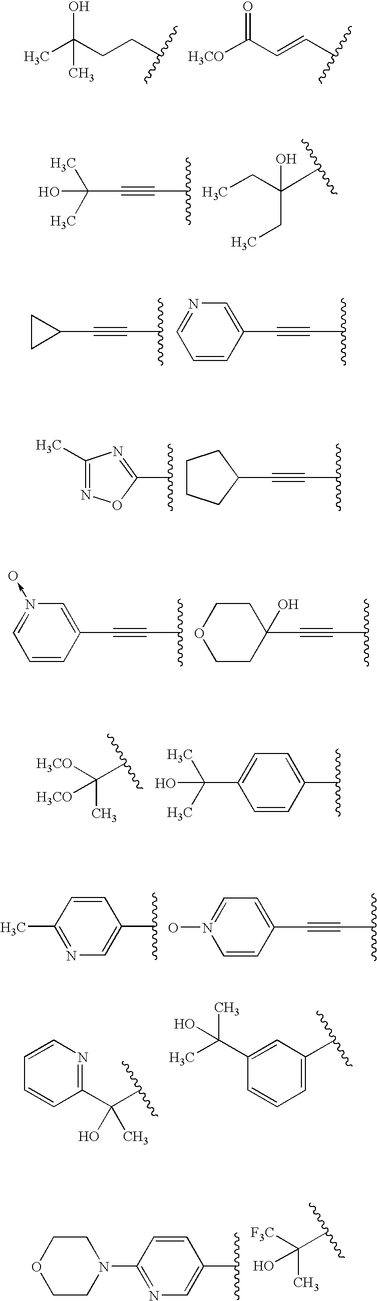

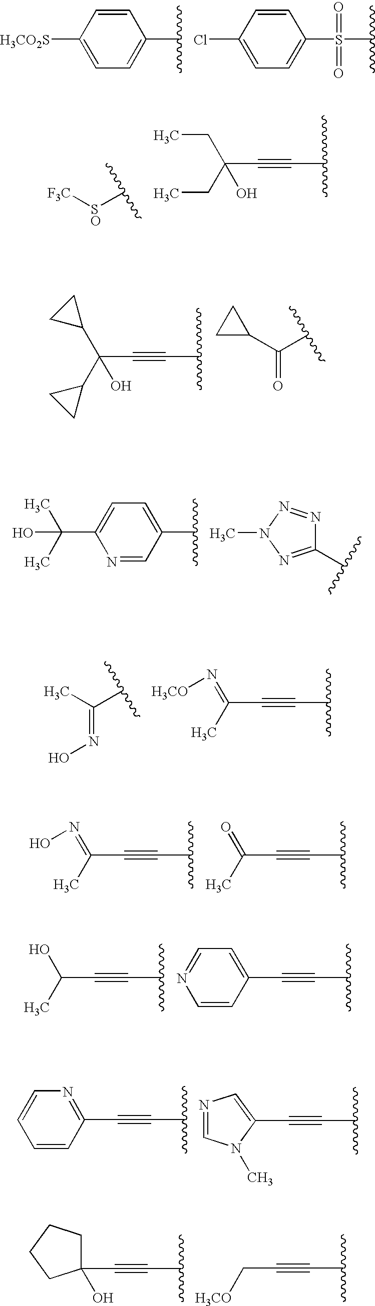

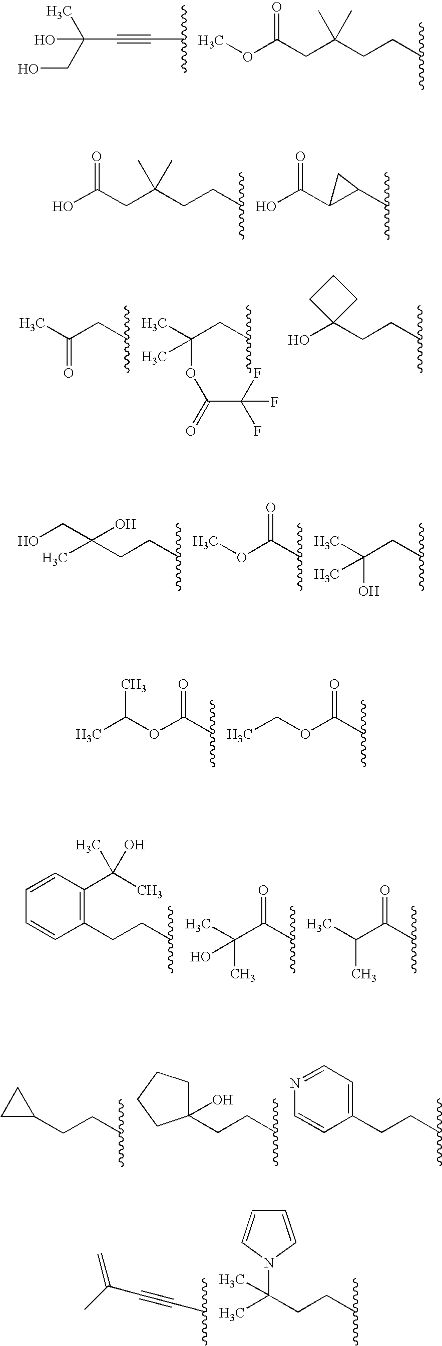

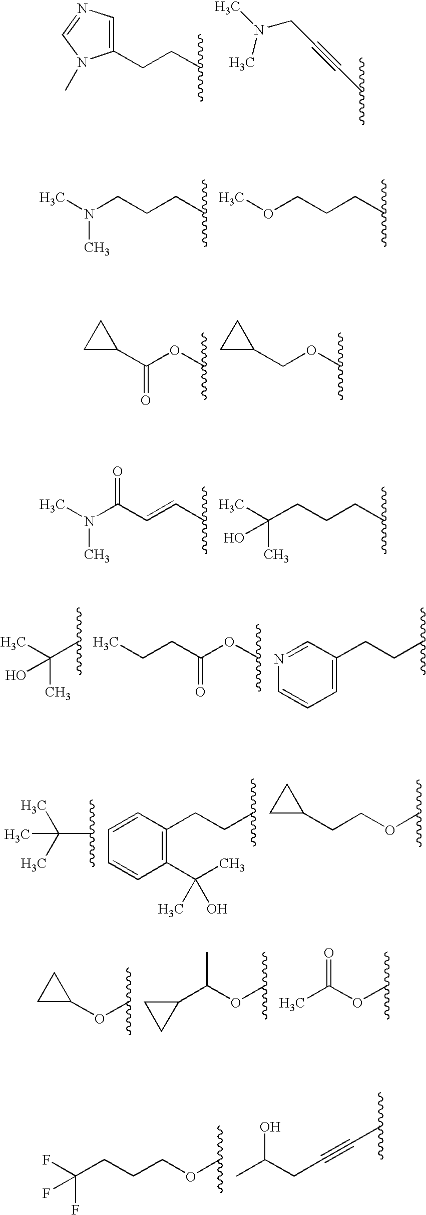

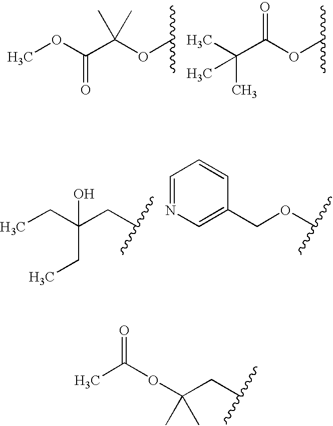

5. The compound according to claim 4 selected from the following table

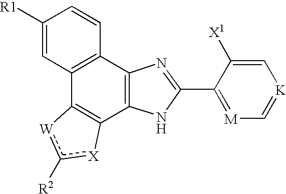

| W | X | R¹ | R² | X¹ | M | K |
|---|---|---|---|---|---|---|
| CH | S | H | H | Cl | CF | CH |
| S | CH | H | Cl | Cl | CF | CH |
| CH | O | H | H | Cl | CF | CH |
| S | CH | H | H | Cl | CF | CH |
| CH | S | CN | H | Cl | CF | CH |
| CH | S | Cl | H | Cl | CF | CH |
| CH | S | Cl | H | CN | CF | CH |
| CH | S | Cl | H | Br | CBr | CH |
| CH | S | Cl | H | CN | CCN | CH |
| CH | 5 | Cl | H | Br | CBr | N |
| CH | S | Cl | H | CN | CCN | N |
| CH | O | Cl | H | CN | CCN | CH |
| CH | O | Cl | H | Br | CBr | N |
| CH | O | Cl | H | CN | CCN | N |
| CH | S | Cl | Br | Cl | CF | CH |
| S | CH | H | Br | Cl | CF | CH |
| CH | S | Cl | Br | CN | CCN | CH |
| CH | S | Cl | 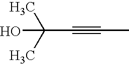 | CN | CCN | CH |
| CBr | S | Cl | Br | Br | CBr | CH |
| CH | S | Cl | Cl | CN | CCN | CH |
| CH | S | Br | Cl | Br | CBr | CH |
| CH | S | Br | Cl | CN | CCN | CH |
| CH | S | 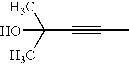 | Cl | CN | CCN | CH |
| CBr | S | Cl | Cl | Br | CBr | CH |
| CBr | S | Cl | Cl | CN | CCN | CH |
| CCl | S | Br | Cl | Br | CBr | CH |
| CCl | S | Br | Cl | CN | CCN | CH |
| CCl | S | 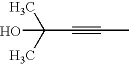 | Cl | CN | CCN | CH |
| CCl | S | 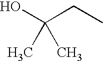 | Cl | CN | CCN | CH |
| CH | S | 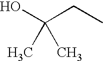 | Cl | CN | CCN | CH |
| CH | S | 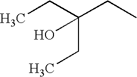 | Cl | CN | CCN | CH |

or a pharmaceutically acceptable salt of any of the aforementioned compounds.

6. The compound according to claim 1 according to Formula C 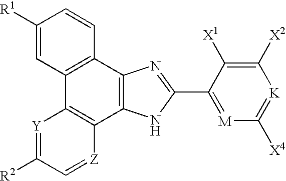
wherein Y and Z are independently selected from the group consisting of: CH and N, or the N-oxide thereof.

7. The compound according to claim 6 wherein
X² and X⁴ are H;
K is CH or CF;
M is —C(X⁵)—; and
X¹ and X⁵ are independently selected from the group consisting of: (1) F; (2) Cl; (3) Br; (4) I; and (5) CN.

8. The compound according to claim 7 wherein
R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl,

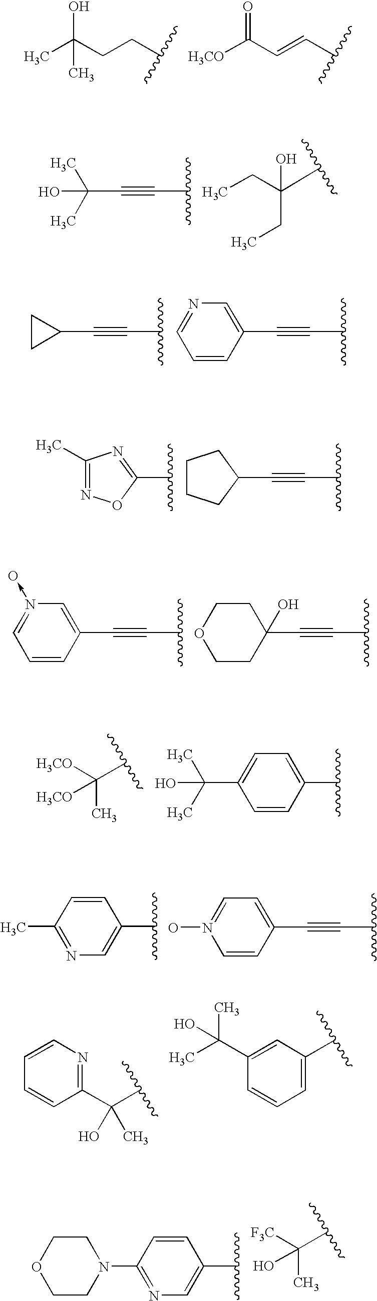

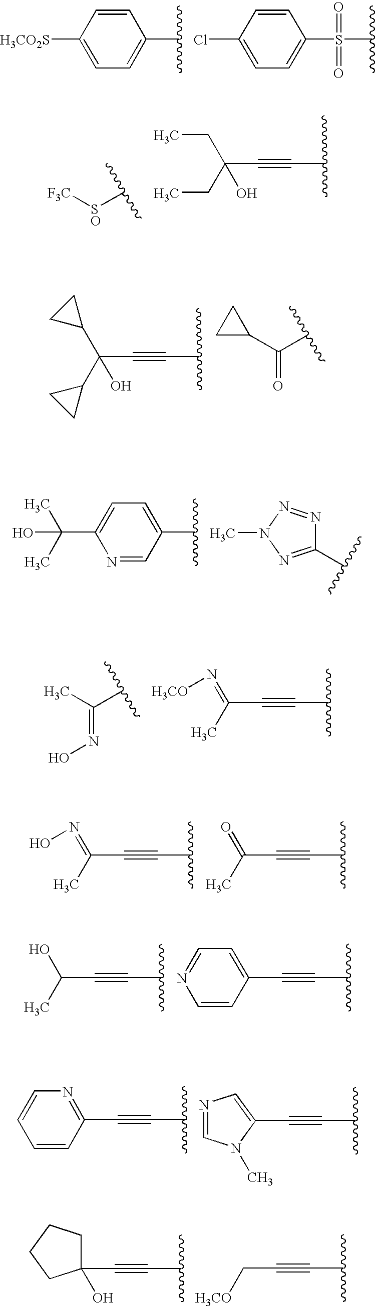

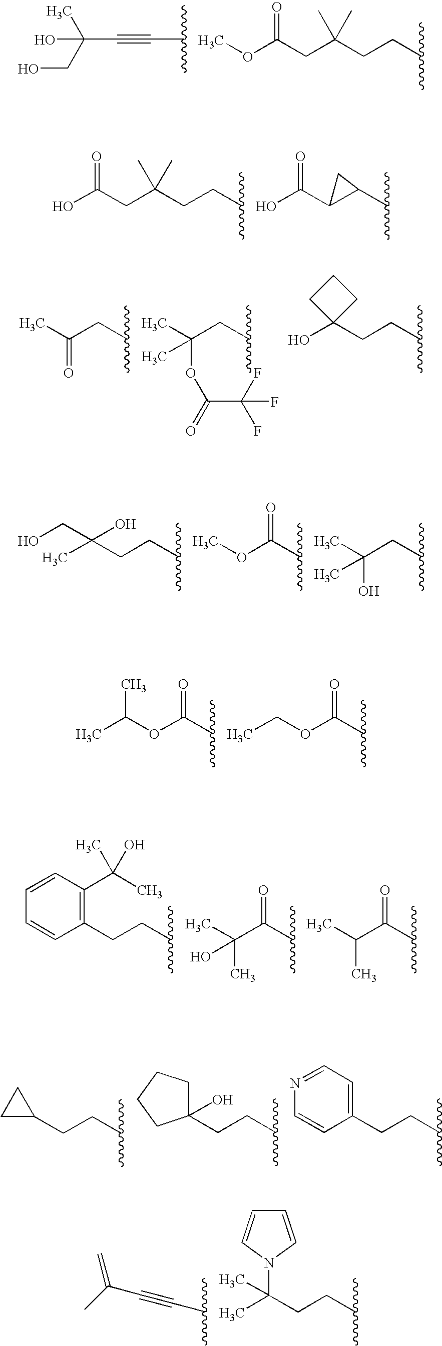

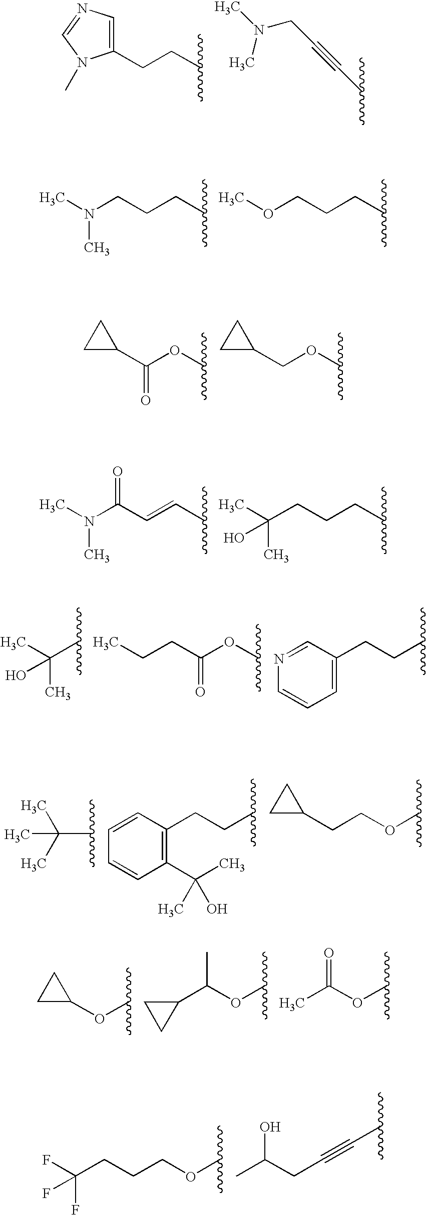

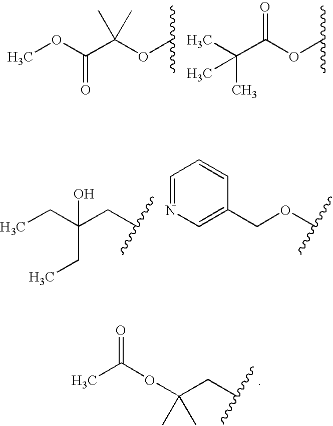

9. The compound according to claim 8 selected from the following table

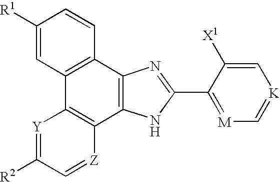

| Y | Z | R¹ | R² | X¹ | M | K |
|---|---|---|---|---|---|---|
| CH | N | H | H | Cl | CF | CH |
| N | CH | H | H | Cl | CF | CH |
| NO | CH | H | H | Cl | CF | CH |
| NH | CH | H | O | Cl | CF | CH |
| N | CH | H | H | Br | CBr | CH |
| N | CH | H | H | CN | CCN | CH |
| NO | CH | H | H | Br | CBr | CH |
| N | CH | H | Cl | CN | CCN | CH |
| N | CH | H | Ph | Br | CBr | CH |
| N | CH | H | Ph | CN | CCN | CH |
| N | CH | OCH3 | Cl | Br | CBr | CH |
| N | CH | OCH3 | Cl | CN | CCN | CH |
| N | CH | 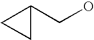 | 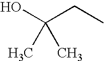 | Br | CBr | CH |
| N | CH | 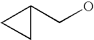 | 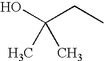 | CN | CCN | CH |
| N | CH | 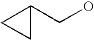 | Cl | CN | CCN | CH |
| N | CH | 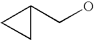 | 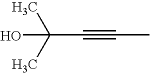 | CN | CCN | CH |
| N | CH | 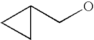 | 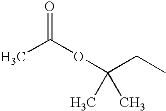 | CN | CCN | CH |
| N | CH | 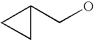 | CN | CN | CCN | CH |
| N | CH | 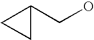 | 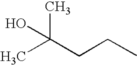 | CN | CCN | CH |
| N | CH | 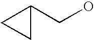 | Cl | CN | CCN | CF |
| N | CH | 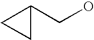 | 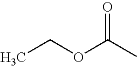 | CN | CCN | CH |
| N | CH | 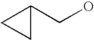 | 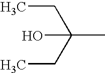 | CN | CCN | CH |
| N | CH | 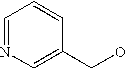 | Cl | CN | CCN | CH |
| N | CH | 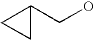 | 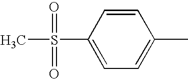 | CN | CCN | CH |

or a pharmaceutically acceptable salt of any of the aforementioned compounds.

10. The compound according to claim 1 wherein:
X¹ is selected from the group consisting of: (1) F; (2) Cl; (3) Br; (4) I; and (5) CN; and
X², X³, X⁴ and X⁵ are independently selected from the group consisting of: (1) H; (2) F; (3) Cl; (4) Br; (5) I; and (6) CN.

11. The compound according to claim 1 wherein M is —C(X⁵)—.

12. The compound according to claim 11 wherein X⁵ is other than H.

13. The compound according to claim 12, wherein X¹ and X⁵ are the same and selected from the group consisting of: (1) F; (2) Cl; (3) Br; (4) I and (5) CN.

14. The compound according to claim 1, wherein at least one of R¹ or R² is present and other than H.

15. The compound according to claim 14, wherein: R¹ and R² are independently selected from the group consisting of: hydrogen, fluoro, chloro, bromo, iodo, cyano, methyl, methoxy, ethyl, vinyl, cyclopropyl, propyl, butyl, —SO₂CF₃, 3-pyridyl, acetyl, phenyl 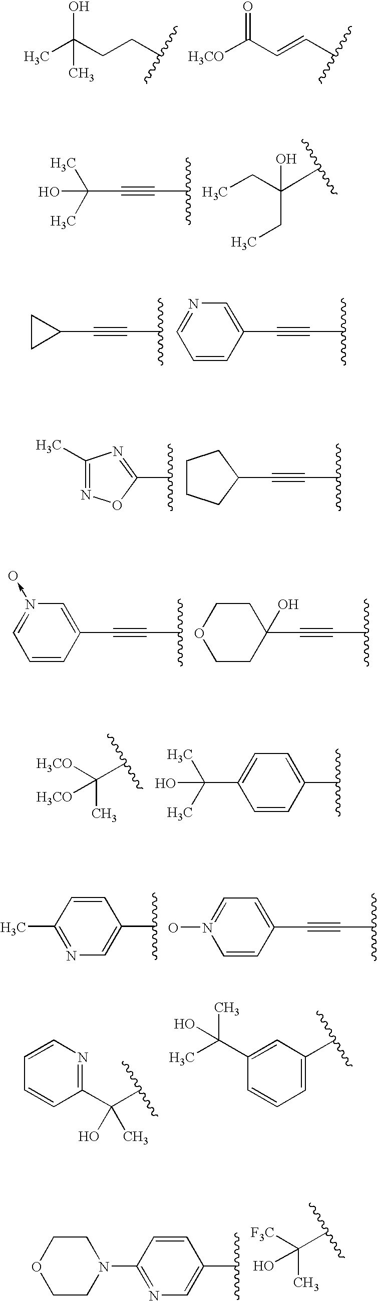 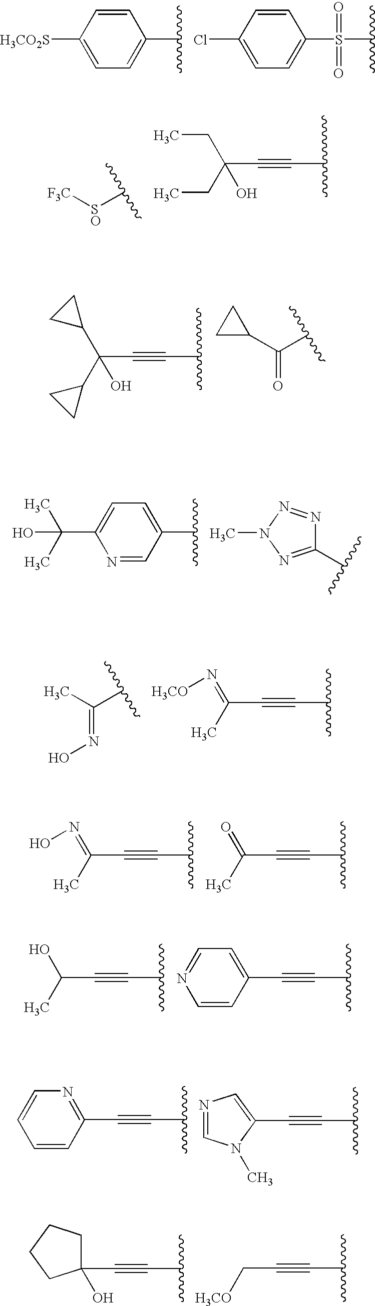 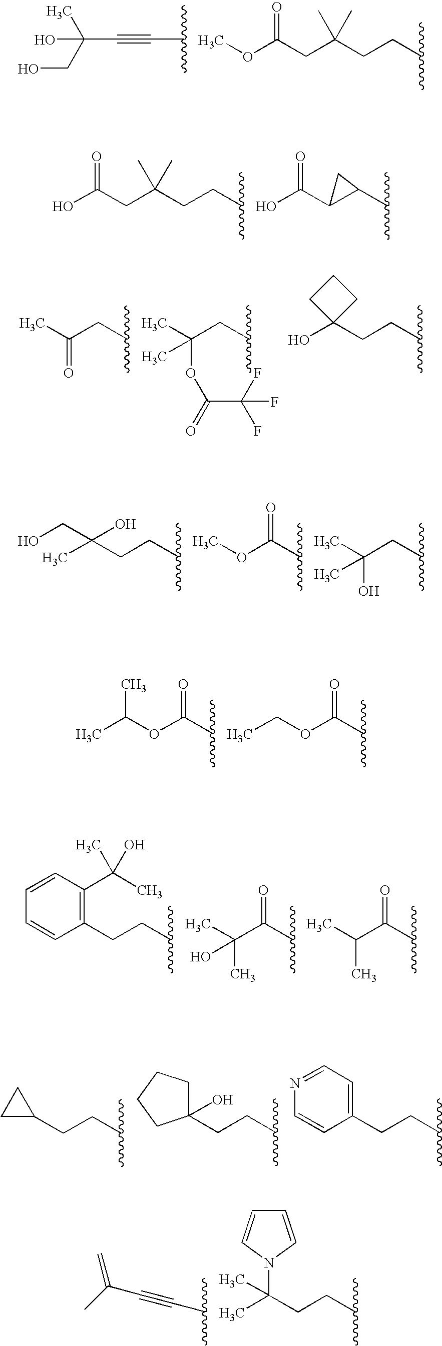 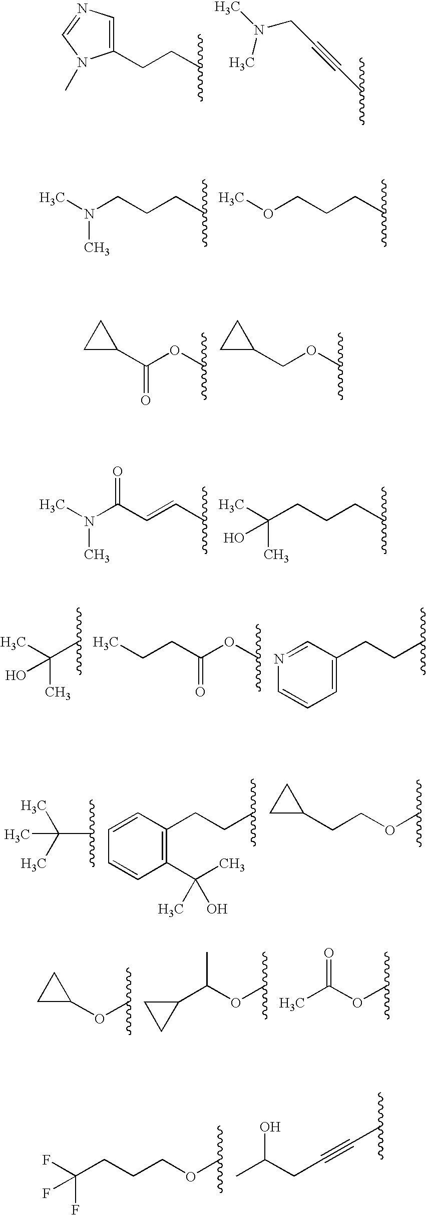 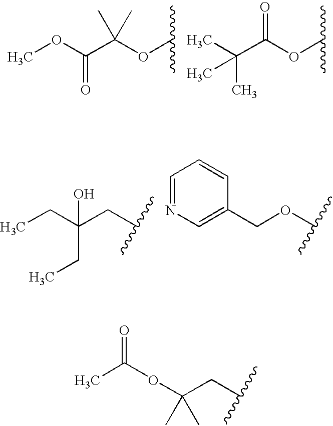
with the proviso that at least one of R¹ or R² is present and other than H.

16. The compound according to claim 1 wherein
A is selected from the group consisting of: aryl and heteroaryl, or a fused analog of any of aforementioned;
B is selected from the group consisting of: aryl and heteroaryl, or a fused analog of any of the aforementioned;
with the proviso that A and B cannot both simultaneously be phenyl.

17. The compound according to claim 1 selected from the following group: 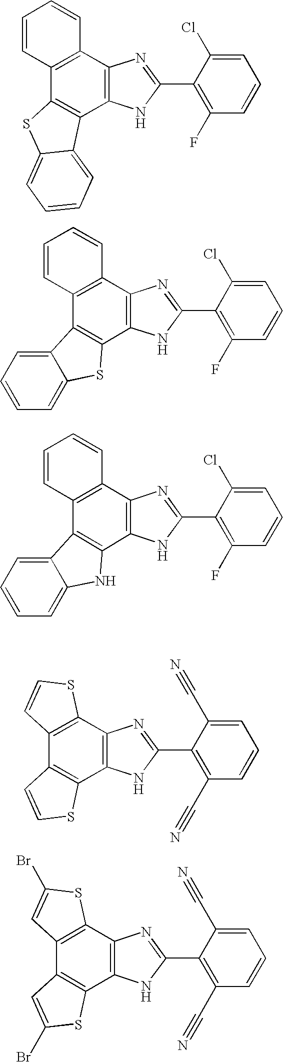
or a pharmaceutically acceptable salt of any of the aforementioned compounds.

18. A pharmaceutical composition comprising a compound according to claim 1 in combination with a pharmaceutically acceptable carrier.

19. A method for treating a microsomal prostaglandin E synthase-1 mediated disease or condition in a human patient in need of such treatment comprising administering to said patient a compound according to claim 1 in an amount effective to treat the microsomal prostaglandin E synthase-1 mediated disease or condition.

20. The method according to claim 19 wherein the disease or condition is selected from the group consisting of: acute or chronic pain, osteoarthritis, rheumatoid arthritis, bursitis, ankylosing sponylitis and primary dysmenorrhea.
